(19) United States
(12) Patent Application Publication
(10) Pub. No.: US 2022/0175848 A1
(43) Pub. Date: Jun. 9, 2022

(54) METHODS AND COMPOSITIONS FOR CELL THERAPY

(71) Applicants: THE BROAD INSTITUTE, INC., Cambridge, MA (US); THE BRIGHAM AND WOMEN'S HOSPITAL, INC., Boston, MA (US)

(72) Inventors: Amit CHOUDHARY, Boston, MA (US); Sreekanth VEDAGOPURAM, Boston, MA (US); Donghyun LIM, Cambridge, MA (US)

(21) Appl. No.: 17/600,799

(22) PCT Filed: Apr. 1, 2020
(86) PCT No.: PCT/US2020/026274

§ 371 Date: Oct. 1, 2021

Related U.S. Application Data
(60) Provisional application No. 62/827,772, filed on Apr. 1, 2019.
(60) Provisional application No. 62/838,152, filed on Apr. 24, 2019.
(60) Provisional application No. 62/838,865, filed on Apr. 25, 2019.
(60) Provisional application No. 62/851,631, filed on May 22, 2019.
(60) Provisional application No. 62/914,345, filed on Oct. 11, 2019.

(51) Int. Cl. A61K 35/39; C12N 15/10; A61P 3/10; C12N 5/071

(52) U.S. Cl. CPC A61K 35/39; C12N 5/0676; A61P 3/10; C12N 15/102

(57) ABSTRACT

An engineered cell is provided that may include one or more polynucleotides inserted into a sequence of a secretory protein of the cell, which may be a β-cell.

### CROSS-REFERENCE TO RELATED APPLICATIONS

This claims the benefit of U.S. Provisional Application No. 62/827,772 filed Apr. 1, 2019, U.S. Provisional Application No. 62/838,152 filed Apr. 24, 2019, U.S. Provisional Application No. 62/838,865 filed Apr. 25, 2019, U.S. Provisional Application No. 62/851,631 filed May 22, 2019, and U.S. Provisional Application No. 62/914,345 filed Oct. 11, 2019. The entire contents of the above-identified applications are hereby fully incorporated herein by reference.

### REFERENCE TO AN ELECTRONIC SEQUENCE LISTING

An ASCII compliant text file, (“BROD-4140WP_ST25.txt”, size 18,214 bytes, created on Mar. 30, 2020) is filed herewith via EFS-WEB; the contents of the electronic sequence listing are herein incorporated by reference in its entirety.

### TECHNICAL FIELD

The subject matter disclosed herein is generally directed to producing cells for transplanting to a patient.

### BACKGROUND

Type 1 diabetes mellitus (T1D) is disease that burdens over 3 million patients and is responsible for over $14 billion in annual healthcare costs in the U.S. There is a significant need for a cure that can regenerate insulin independence and restore the patient's quality of life. β-cell transplantation shows promise, however poor graft survival due to alloimmune and autoimmune rejection and engraftment inefficiency prevents sustained therapeutic effects, and it suffers from a striking initial graft loss of 55% to 70%. Global immunosuppressants can decrease islet rejection, but there is increased risk for opportunistic infections. Co-transplantation with MSCs has been examined to prolong the functionality of islet grafts through their ability to alter nearby cytokine profiles including increased production of IL-10, via interaction with dendritic cells and generation of CD4⁺ T cells. However, administering multiple cell types is complex and MSCs have batch-dependent variability, and their survival is short-lived. There is a need for donor cells that trigger minimum immune response and have sustained survival time.

### SUMMARY

In certain example embodiments, an engineered cell is provided comprising one or more polynucleotides encoding: one or more secreted proteins, or a functional fragment thereof. In embodiments, the one or more secreted proteins or functional fragments thereof are inserted in one or more endogenous genes of the cell, the one or more endogenous genes encoding a secretory protein or peptide, optionally, a co-expressed protein that enhances the expression, stability or biological activity of the secreted protein.

In embodiments, the secreted protein is an immunomodulatory protein, an anti-fibrotic, a hormone, anti-microbial or a protein or peptide that promotes tissue regeneration. The secreted protein may be an immunomodulatory protein and the co-expressed protein an anti-fibrotic protein in some instances.

In embodiments, the immunomodulatory protein is a cytokine, in certain embodiments, IL-2, IL-4, IL-6, IL-10, IL-15, or IL-22.

The secreted protein is, in certain embodiments, an anti-fibrotic protein, optionally wherein the anti-fibrotic protein is a peroxisome proliferator-activated receptor. The secreted protein may comprise a protein or peptide that promotes tissue regeneration, optionally Reg1A, Reg1B, Reg2, Reg3A, Reg3g, and/or Reg 4, or a combination thereof.

In embodiments, the secreted protein is a hormone. In embodiments, the hormone is insulin, or the hormone is a neuroendocrine hormone, optionally selected from thyrotropin-releasing hormone, corticotropin-releasing hormone, histamine, growth hormone-releasing hormone, somatostatin, gonadotropin-releasing hormone, serotonin, dopamine, neurotensin, oxytocin, vasopressin, epinephrine, and norepinephrine.

In embodiments, the secreted protein is an anti-microbial, optionally wherein the anti-microbial is α-defensin HD-6, HNP-1 and β-defensin hBD-3

The cell in some embodiments is a eukaryotic cell. In certain embodiments, the cell is a human cell or a plant cell. The cell can be an α a cell or β cell, an L cell, a stem cell, or a primary cell. The cell can be an endocrine cell, in an aspect, a pancreatic endocrine cell. In certain embodiments, the cell is a pancreatic β cell. In some embodiments, the pancreatic β cell is differentiated from a progenitor in vitro.

In embodiments, the cell further comprises one or more polynucleotides encoding a protein promoting pancreatic β cell regeneration. In some embodiments, the protein is a cytokine, in some instances, IL-22. In embodiments, the protein promoting pancreatic β cell regeneration upregulates one or more genes promoting pancreatic β cell regeneration. In embodiments, the one or more genes promoting pancreatic β cell regeneration is Reg1, Reg2, or a combination thereof.

The cells can further comprise one or more polynucleotides encoding interference RNA. In embodiments, the interference RNA is siRNA. In embodiments, the interference RNA suppresses TGF-β, colony-stimulating factor 1, or a combination thereof.

The cells comprising one or more inserted polynucleotides may comprise one or more protease cleavage sites. In embodiments, the one or more inserted polynucleotides comprise a protease cleavage site on each end. The secretory protein or peptide of the cell can comprise c-peptide.

Methods of treating a disease are disclosed, comprising administering a cell of as disclosed herein to a subject in need thereof. In embodiments, the disease is diabetes.

Methods of making the cells disclosed herein are provided, comprising obtaining s cell from an organism; editing the cell to insert one or more polynucleotides, wherein insertion is in-frame with the secretory protein. In embodiments, editing the cell is with a nucleic acid editing system. In embodiments, the nucleic acid editing system is selected from a CRISPR nucleic acid editing system, zinc finger nuclease, TALEN, meganuclease, or a Semisynthetic Genome Editing Multifunctional system (Syngem). In embodiments, one or more polynucleotides are inserted in the exon portion of the secretory protein in the cell. In embodiments, the peptide is a c-peptide.

The step of editing can be performed ex-vivo. The methods may further comprise the step of inserting the cell into an organism. In embodiments, the organism from which the cell is obtained and in which the cell is inserted is the same organism. Inserting the cell can be performed by infusion. In embodiments, the step of editing the cell comprises using the CRISPR editing system comprising a gRNA that targets the c-peptide in the middle region of the c-peptide.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1B: (FIG. 1A) An exemplary SynGEM. (FIG. 1B) A HiBiT assay for HDR-mediated knock-in of the 33-nt DNA fragment.

FIGS. 2A-2C: (FIG. 2A) Design for knock-in of HiBiT and IL-10 in c-peptide region of rat preproinsulin. The design shows 3 insertion sites, for each of which two ssODNs were designed, one with single and the other with two flanked prohormone convertase 2 (PC2) cleavable sequences. (FIG. 2B) Optimization of gRNA and ssODN to get high knock-in signal in INS-1E cells. Bars at 1A, 2A, and 3A represent signals when ssODNs with one PC2 site were used, and bars at 1B, 2B, and 3B show signals when ssODNs with two PC2 sites were used. (FIG. 2C) Secretion of IL-10 from the IL-10 knock-in INS-1E cells was quantified from the supernatant media using ELISA.

FIG. 3 shows results of a knock-in of native IL-10, or a ssODN of IL-10 with mutation R125A in c-peptide region of cells using gRNAa.

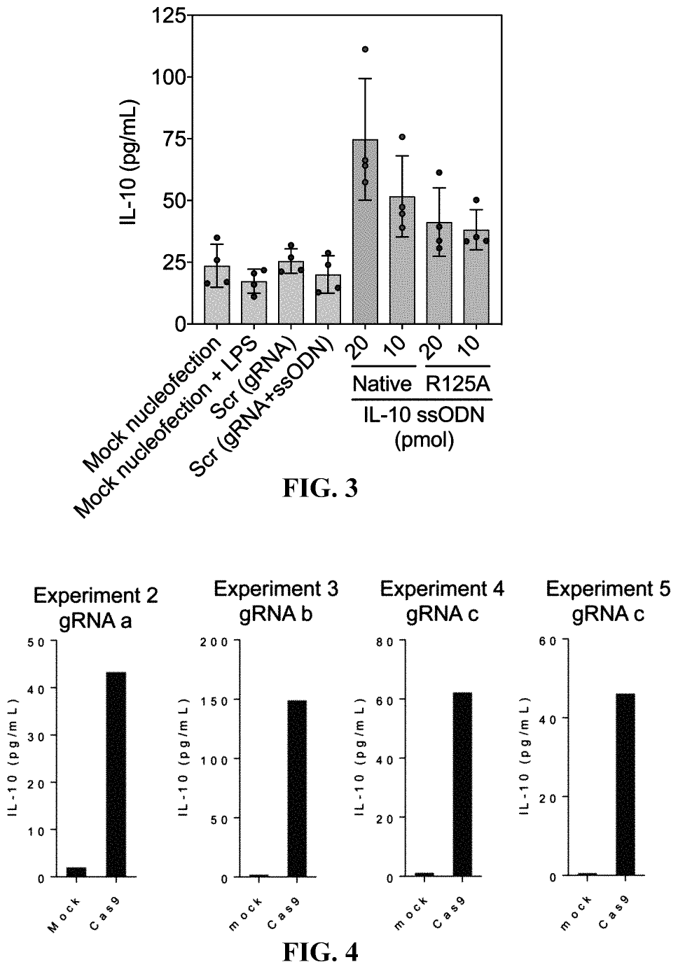

FIG. 4 shows results of further experiments of INS-1E genome editing to secrete IL-10 using several different gRNAs, gRNAa (used in FIG. 3), gRNAb and gRNAc.

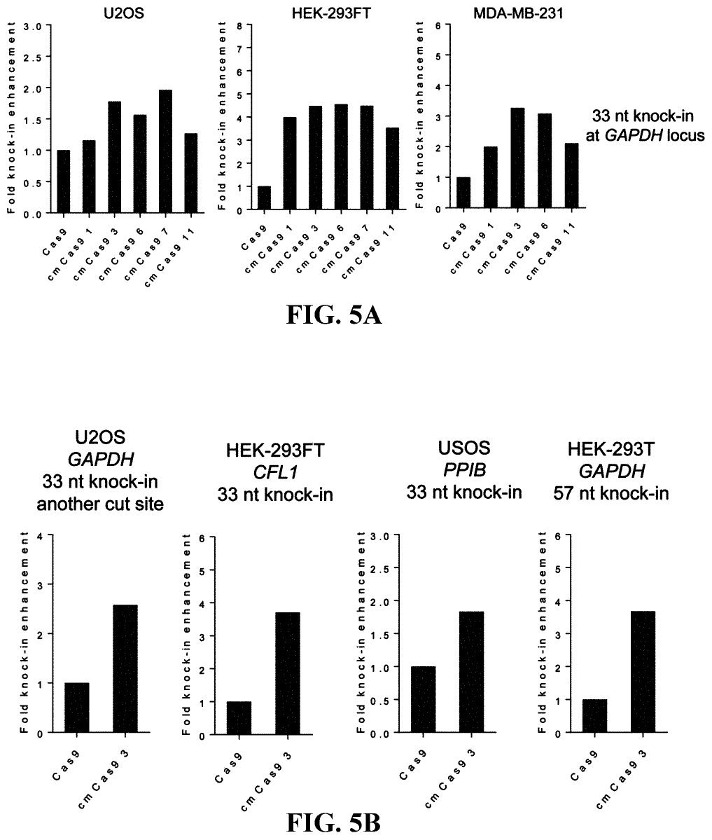

FIG. 5A includes results showing chemically modified Cas9s, (cmCas9s) increases HDR efficiency in multiple cell lines: U2OS left panel, HEK293FT middle panel; MDA-MB-231 right panel; FIG. 5B shows cmCas9 boosts HDR at various genomic loci in a variety of cells: U2OS GAPDH 33 nt knock in; HEK-293FT CFL1 33 nt knock-in, U205, PPIB 33 nt knock-in, and HEK-293T GAPDH 57 nt knock-in.

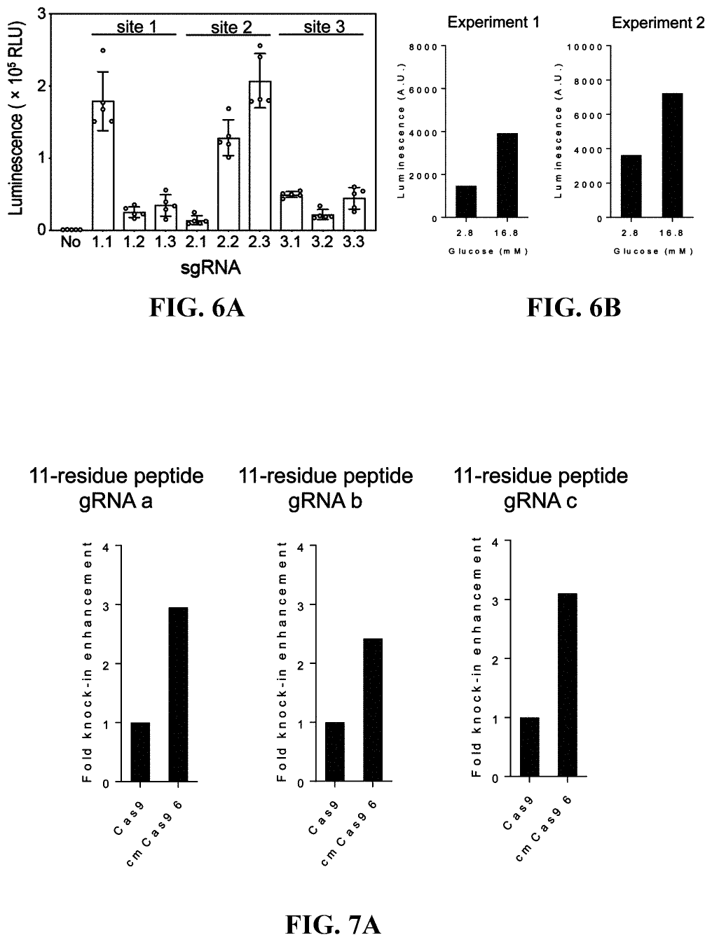

FIG. 6A shows results of INS-1E genome editing to secrete an IL-10 residue peptide, including insertion site and gRNA screening; FIG. 6B shows results of two experiments showing glucose-induced peptide secretion.

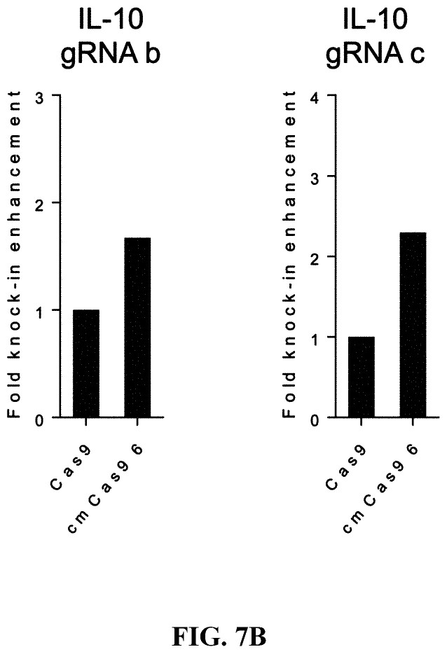

FIG. 7A shows results of cmCas9 enhances precise genome editing, 11-residue peptide using gRNAa, gRNAb and gRNAc; FIG. 7B shows results of IL-10 using gRNAb and gRNAc.

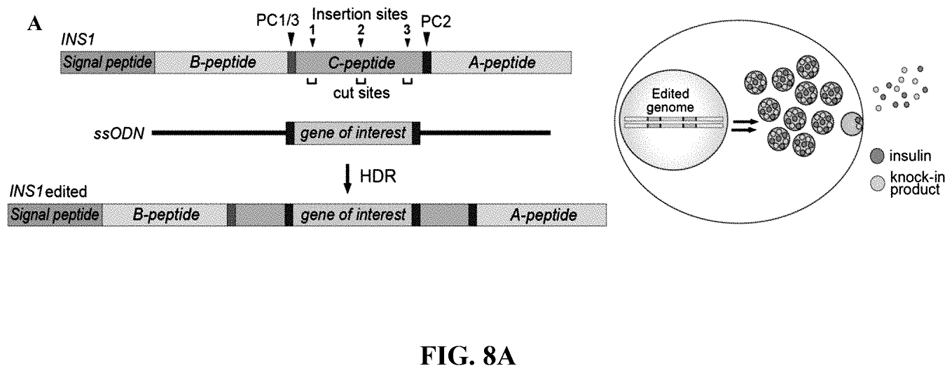

FIG. 8A-FIG. 8F—FIG. 8A shows a schematic of the genome editing in INS1 locus of INS-1E cells to exploit insulin processing and secretion pathway. Engineered cells can secrete exogenous gene product together with insulin. FIG. 8B INS-1E cells were engineered to secrete the 11-residue HiBiT peptide. Multiple gene insertion sites and DNA break sites were investigated. All data from two biological replicates are shown. FIG. 8C Glucose-stimulated HiBiT peptide secretion demonstrates the knock-in at the INS1 locus. All data from five technical replicates are shown. FIG. 8D INS-1E cells were engineered to secret IL-10. All data from three technical replicates are shown. FIG. 8E-8F Cas9-ssODN conjugates enhanced the secretion of (8E) HiBiT peptide and (8F) IL-10. All data from biological replicates are shown.

FIG. 9—(left) A modular design strategy to functionalize Cas9. (right) a schematic of genome editing of the insulin gene in a β-cell to exploit the insulin processing and secretion pathway. Engineered cells can secrete IL-10 together with insulin.

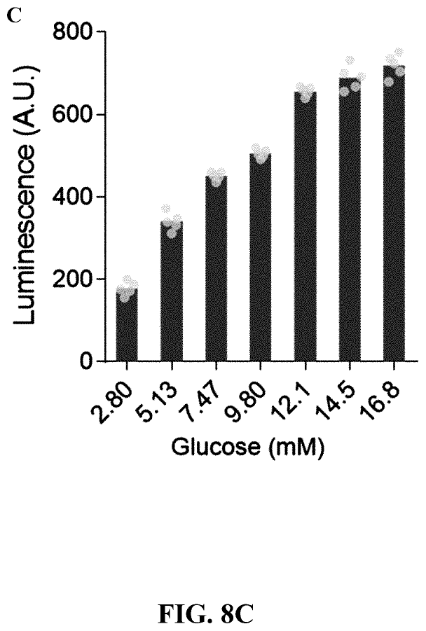

FIG. 10A-FIG. 10E—Schematic of the HiBiT assay to check the HDR-mediated knock-in of the 33-nt DNA fragment. FIG. 10A General gRNA and ssODN design strategy for HDR-based HiBiT sequence knock-in right before the stop codon of the gene of interest.

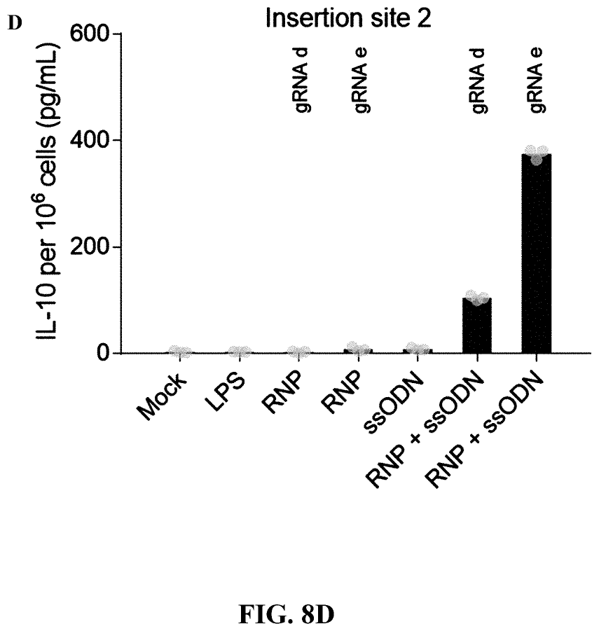

FIG. 10B The knock-in results in the expression of a fusion protein having a C-terminal HiBiT tag, which is a small fragment of the NanoLuc luciferase. When an excess amount of the other fragment of NanoLuc (LgBiT) is supplied, a fully functional NanoLuc is reconstituted. The resulting luminescence signal is proportional to the HDR efficiency. FIG. 10C Design strategy for HiBiT knock-in at the GAPDH locus. FIG. 10D Design strategy for HiBiT knock-in at the PPIB locus or FIG. 10E the CFL1 locus.

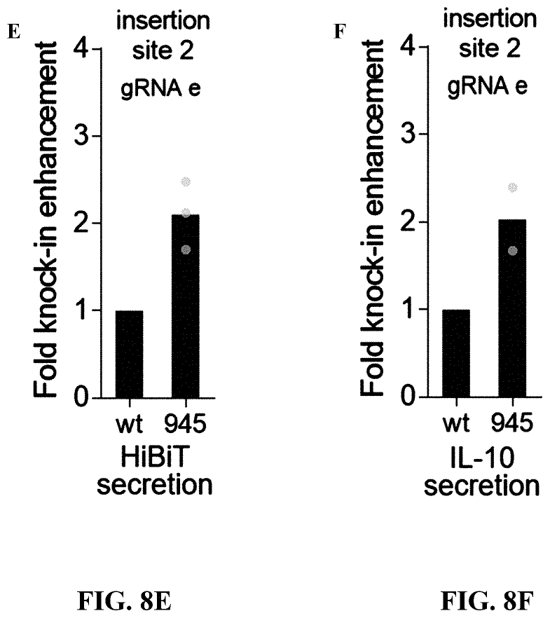

FIG. 11—Glucose-stimulated HiBiT peptide secretion from edited INS-1E cells in independent experiments. All data points from technical replicates are shown.

FIG. 12—IL-10 secretion from edited INS-1E cells in an independent experiment. All data points from technical replicates are shown.

FIG. 13A-FIG. 13B—Confirmation of IL-10 knock-in by PCR. FIG. 13A Primers specific for knock-in sequence were used. FIG. 13B Genomic DNA was extracted from cells exhibiting different IL-10 secretion levels, and PCR was performed using two different primer sets followed by agarose gel electrophoresis and ethidium bromide staining. Numbers in parentheses show IL-10 concentration form the cell culture supernatant. Correct incorporation of IL-10 was confirmed by Sanger sequencing.

FIG. 14A-FIG. 14D—Cas9-ssODN conjugate enhanced precision genome editing in INS-1E cells. Both HiBiT knock-in and IL-10 knock-in were promoted by Cas9-ssODN conjugation when two different gRNAs were tested. Unlabeled wildtype (wt) Cas9 and Cas9-adaptor labeled at residue 945 were used. All data from biological replicates are shown.

FIG. 15—Electrophoretic mobility shift assay to check the binding between Cas9-adaptor conjugates and long ssODNs for IL-10 knock-in. The specific Cas9-ssODN complex was observed only when both Cas9 and ssODN contained the complementary adaptor sequences. The lanes are all from a single gel. Unlabeled wildtype (wt) Cas9 and Cas9-adaptor labeled at residue 945 were used.

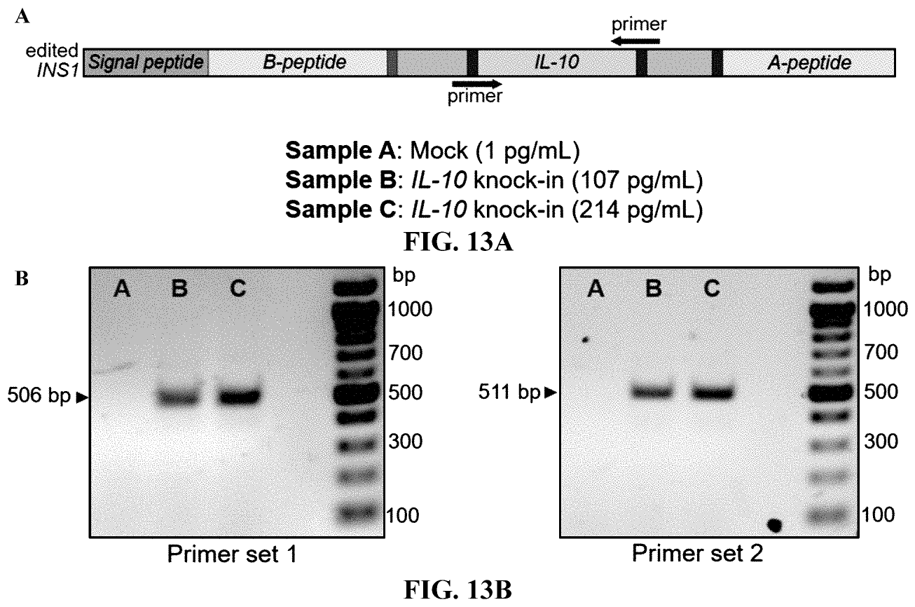

FIG. 16—Glucose-stimulated peptide secretion. Edited cells were grown in a large scale and GSPS study was done in a 24-well plate.

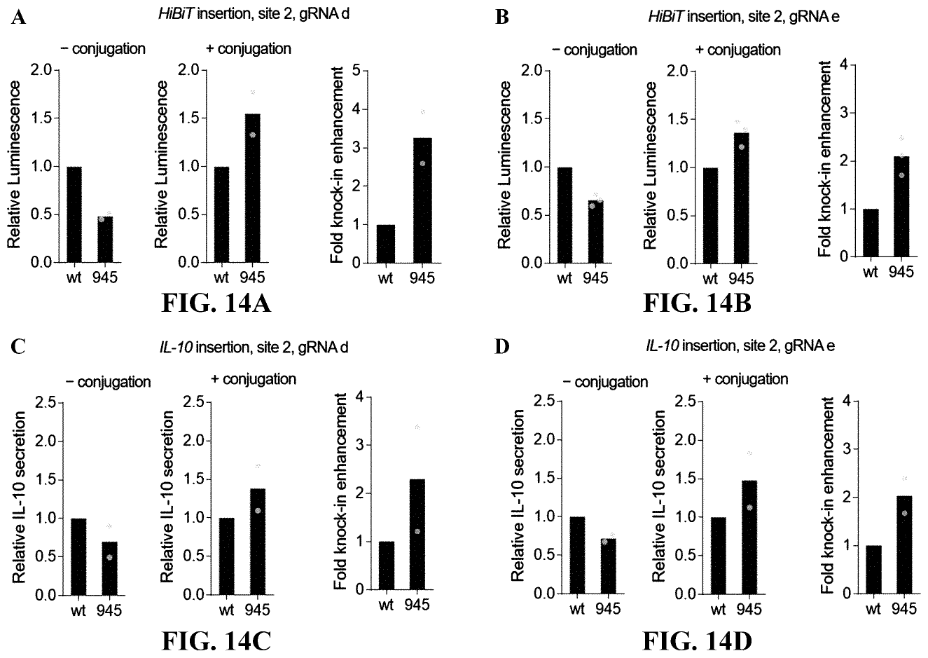

FIG. 17—Glucose-stimulated peptide secretion. Based on HiBiT screening and off-target prediction, insertion site 2 and gRNA d and e were selected.

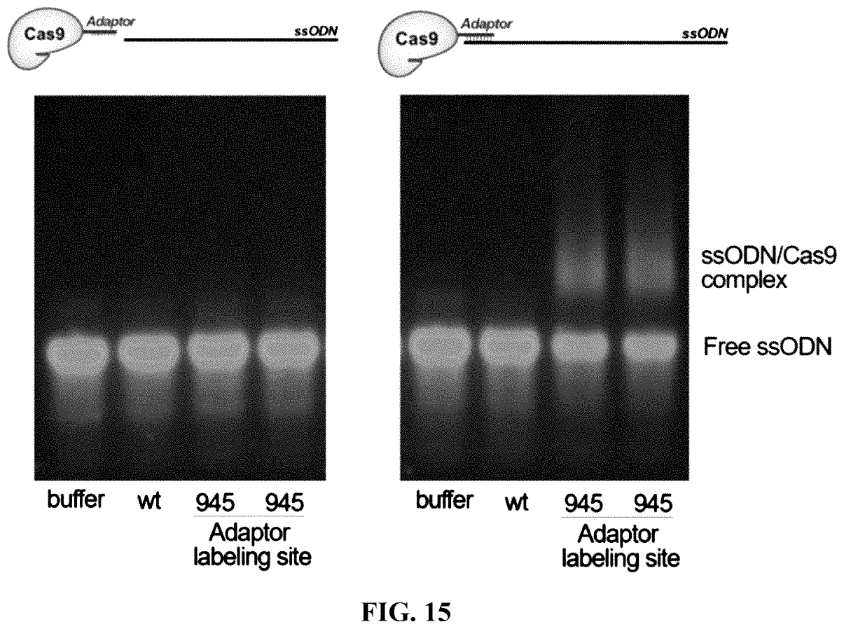

FIG. 18—Cas9-ssODN conjugates for IL-10 insertion

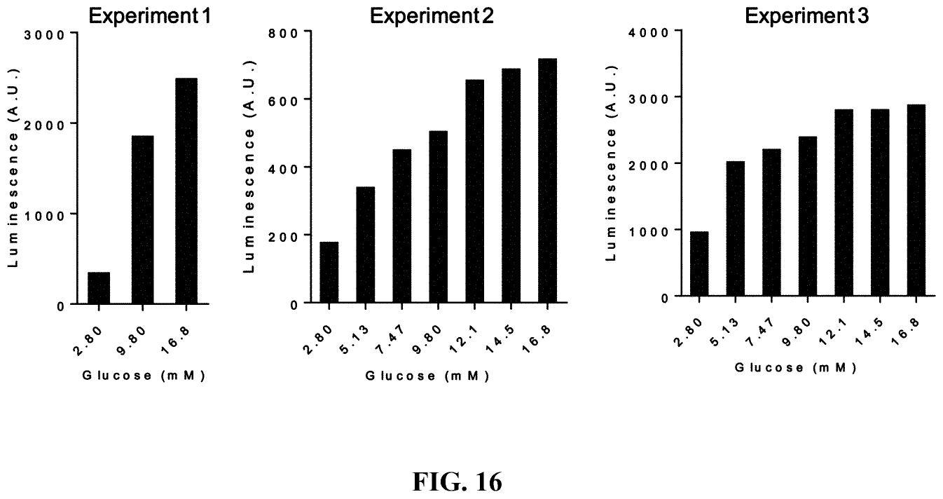

FIG. 19—Confirmation of the correct knock-in. PCR amplification at the INS1 locus.

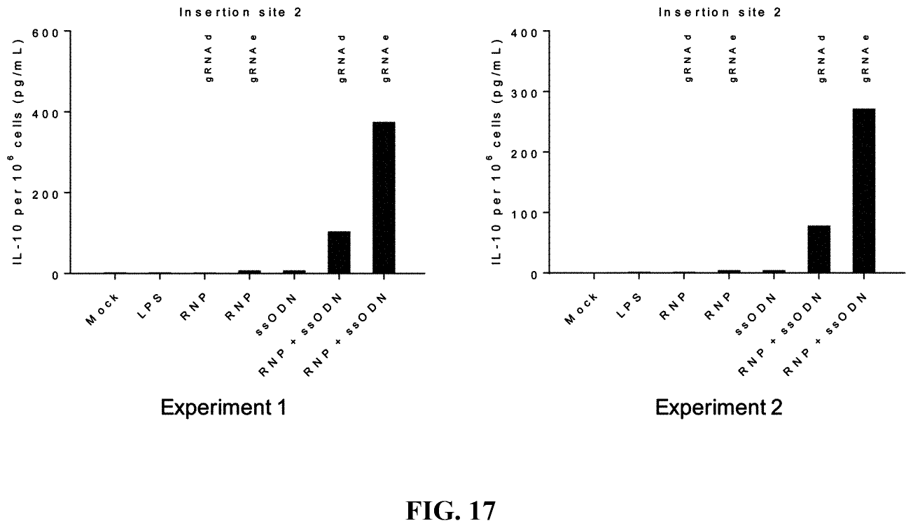

FIG. 20—Genome editing of pancreatic α-cells: mGcg locus

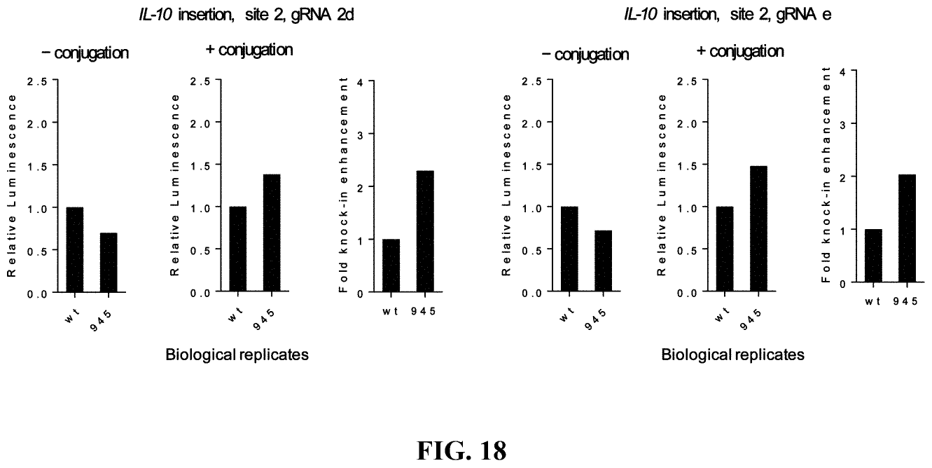

FIG. 21—Genome editing of pancreatic α-cells: mGcg locus

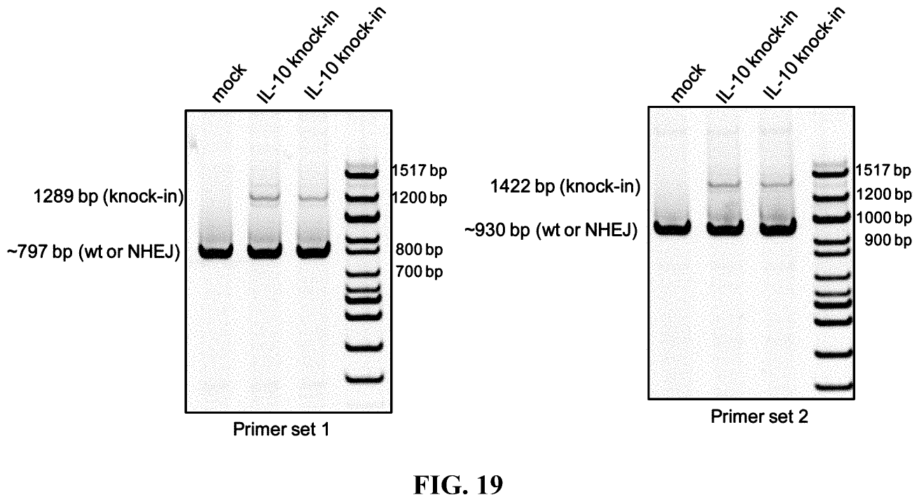

FIG. 22—HiBiT secretion from pancreatic α-cells: mGcg locus

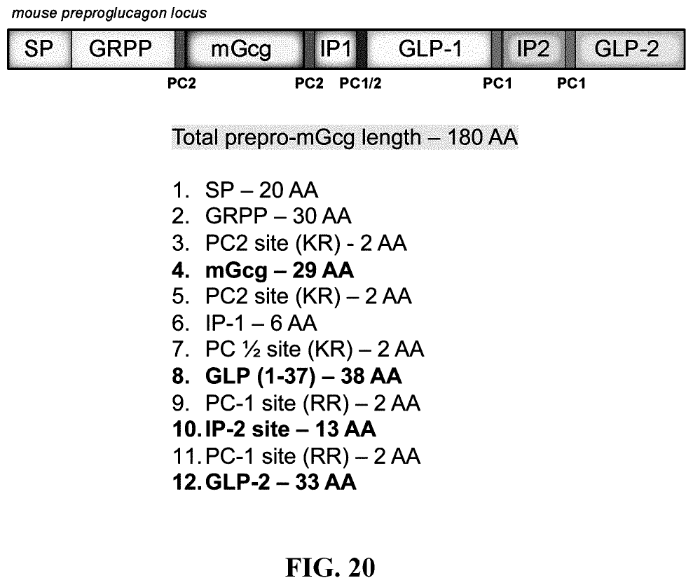

FIG. 23—HiBiT secretion from mGcg locus: glucose-stimulated insulin secretion (GSIS)

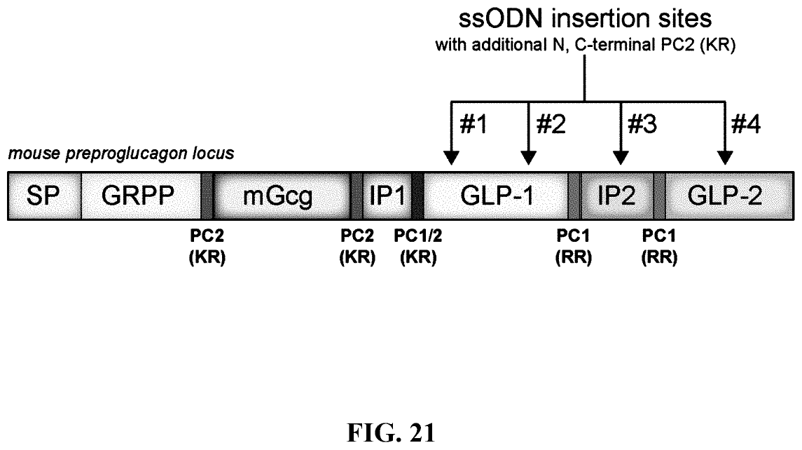

FIG. 24—HiBiT secretion from pancreatic α-cells: mGcg locus

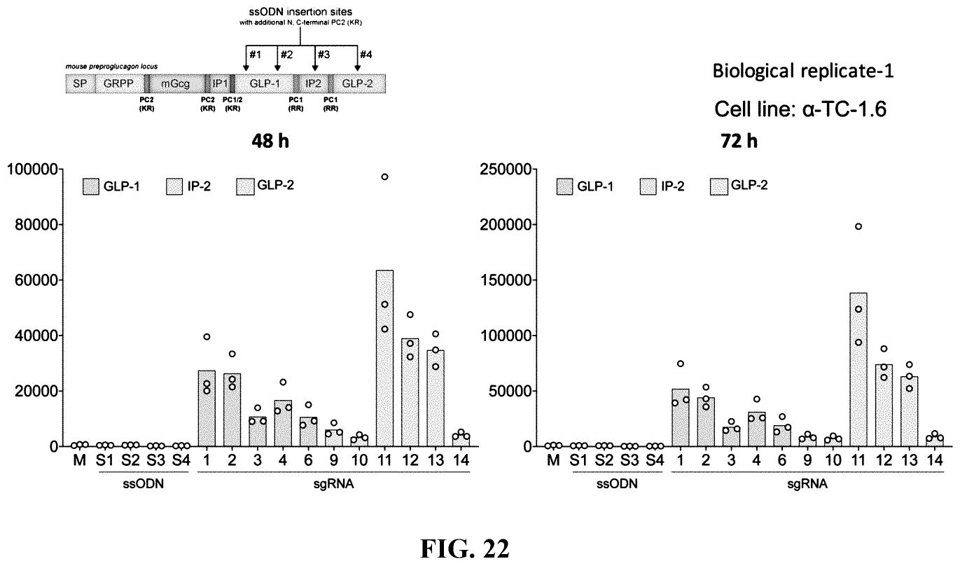

FIG. 25—HiBiT secretion from mGcg locus: GSIS

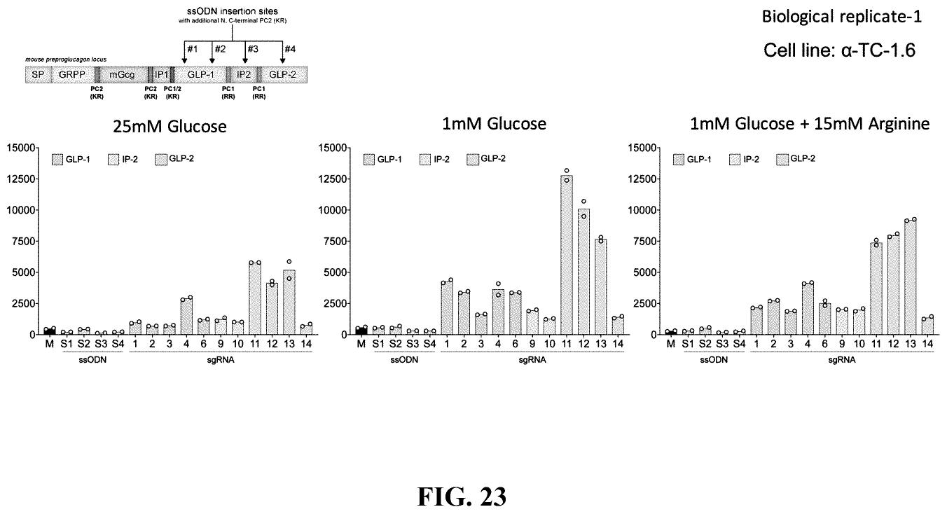

FIG. 26A-26B—Beta cell engineering. FIG. 26A Overview of insulin gene processing and engineering of n-cells; FIG. 26B Screening of multiple genomic loci for the efficient secretion of small peptides and IL-10.

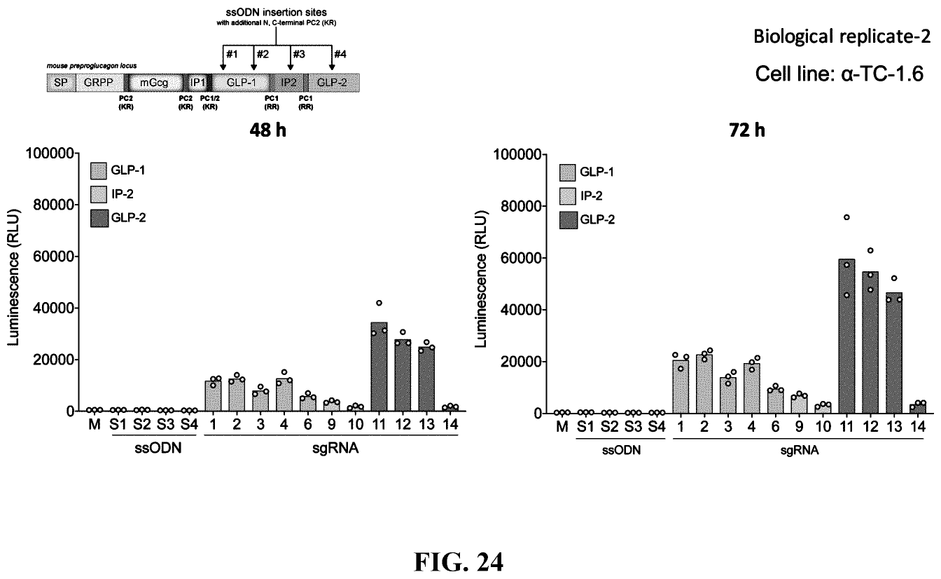

FIG. 27—Alpha cell engineering screening of insertion site 4 in αTC-1.6.

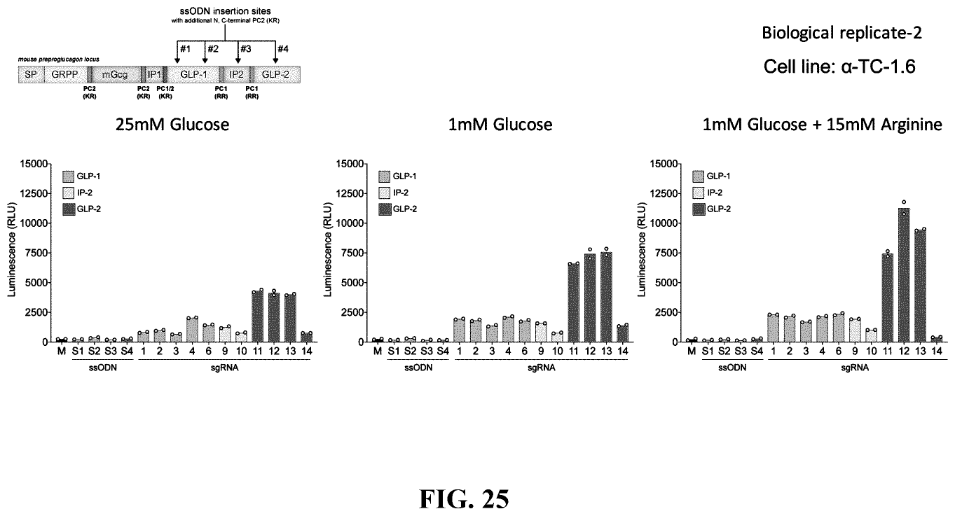

FIG. 28—L-cell engineering: Hijacking the glucagon-like peptide-1 (GLP-1 (secretion machinery screening of multiple genomic loci for the secretion of small peptides in NCI-H716.

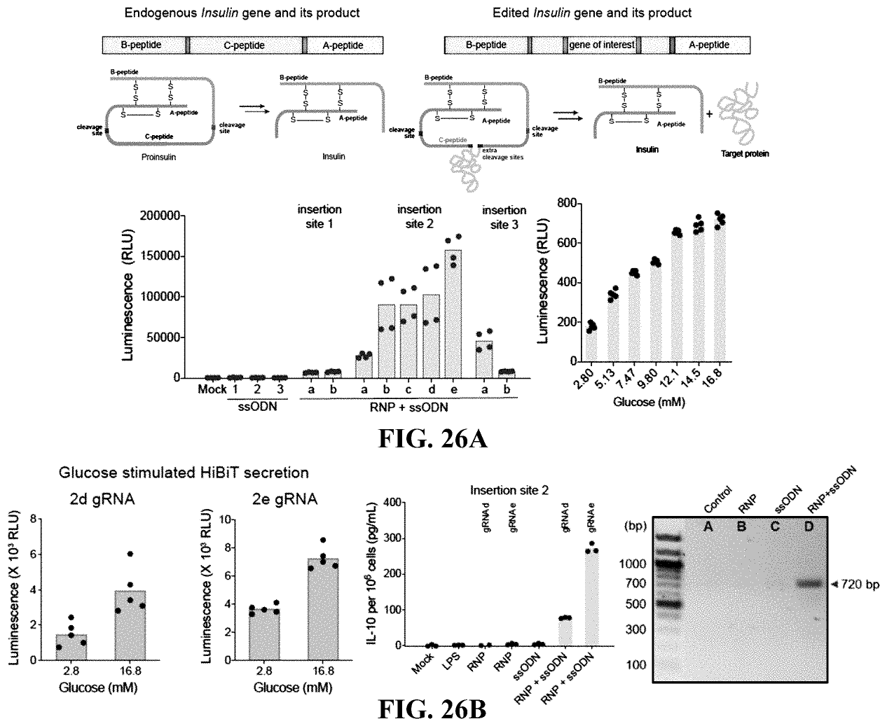

FIG. 29A-29B—Induced Pluripotent Stem Cell (iPSC) engineering: FIG. 29A Editing of insulin and FIG. 29B glucagon locus

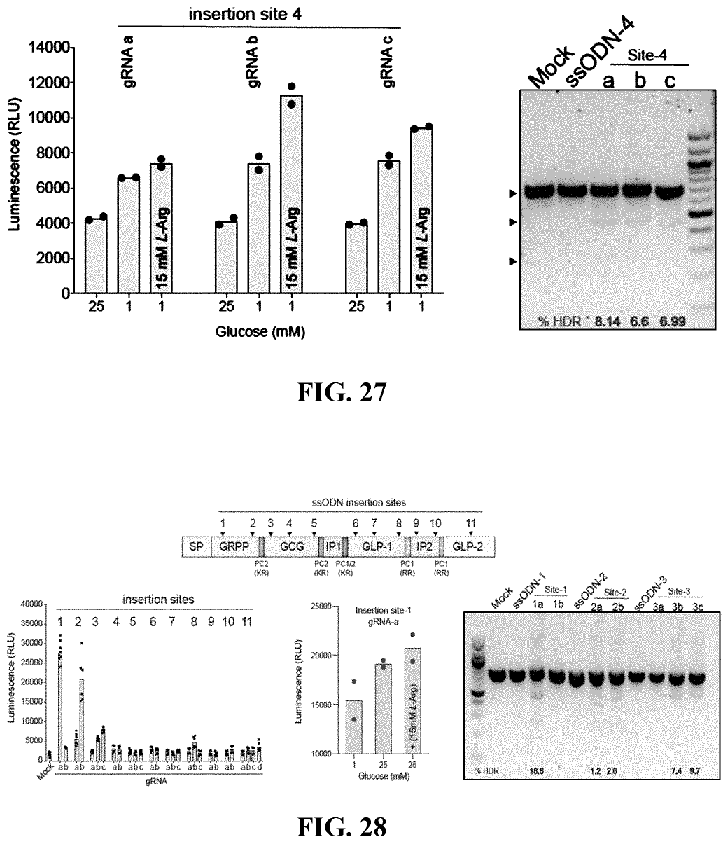

FIG. 30—Future directions include edited stem cells with directed differentiation to stem cell derived endocrine cells and subsequent implantation

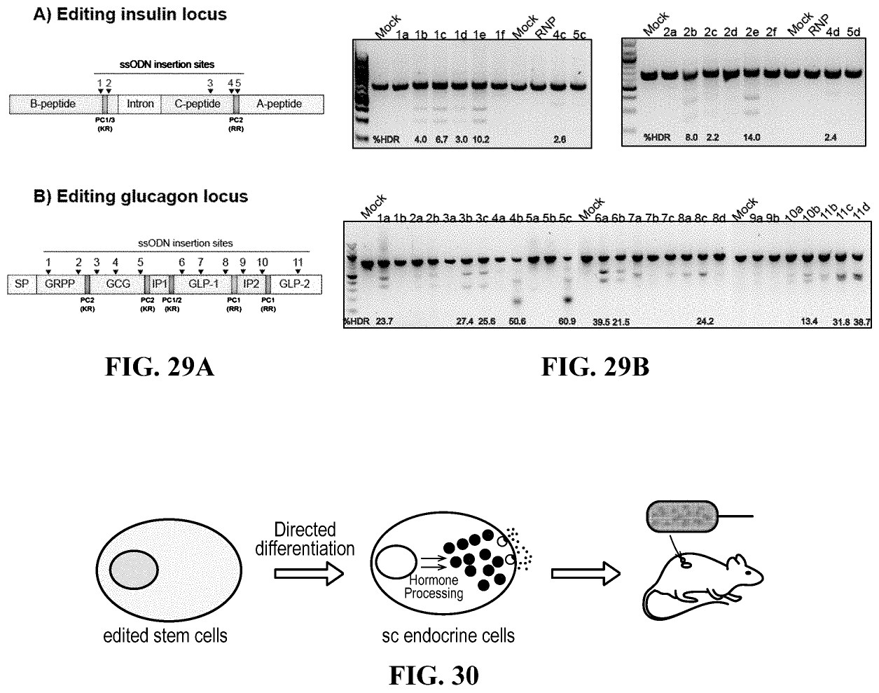

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for engineered cells that express an inserted polynucleotide. In particular, the present disclosure provides cells comprising one or more polynucleotides that are inserted in an endogenous gene in the cell. In embodiments, the endogenous gene encodes a secretory protein or peptide. Methods of making and using the cells are also provided. In an embodiment, the engineered cells are used for cell therapy. In certain instances, the cells may express immunosuppressive proteins, anti-fibrosis proteins, hormones, factors for promoting cell survival and regenerations, interference RNAs that suppress immunogenic pathways, or a combination thereof.

The engineered cells may comprise one or more polynucleotides encoding one or more proteins. In certain embodiments, the polynucleotides are inserted in an endogenous gene (e.g., a gene encoding a secretory protein). While a variety of methods may be used, in one embodiment, a semi-synthetic genome editing multifunction system (SynGEM) is used, which utilizes a Cas protein engineered for precise insertion of the polynucleotides. The Cas protein may be chemically attached a ssODN, an inhibitor of NHEJ, an inhibitor of p53, an adapter oligonucleotide, or a combination thereof.

### Engineered Cells

The present disclosure provides engineered, non-naturally occurring cells. The cells can be from any organism, in preferred embodiments, the cell is from a eukaryote. In certain embodiments, the cell is a human cell or plant cell. The cell may be a stem cell, immune cell, and/or primary cell. In some embodiments, the cell is an α a cell, L cell, or β cell. In certain embodiments, the cell is an endocrine cell, for example, a pancreatic cell. The engineered cells may be used as a transplant donor in cell therapy. In a preferred embodiment, the cells may express one or more inserted genes that, when inserted into an organism, reduce immune response in the organism and prolong survival and function of the inserted cell. In embodiments, the cells express one or more proteins or peptide encoded by the inserted polynucleotide. In embodiments, the one or more inserted polynucleotides are inserted in the exon of a secretory protein such that the encoded protein or peptide is secreted.

### Inserted Polynucleotides

The cells herein may comprise one or more inserted polynucleotides encoding one or more secreted proteins, or functional fragments thereof, inserted in one or more endogenous genes of the cell. The one or more inserted polynucleotides or functional fragments thereof encode a secretory protein, or may encode a non-secretory protein. The polynucleotide which has been introduced into the cell may comprise an endogenous or exogenous polynucleotide, including a sequence homologous to a sequence in the cell into which it is introduced but in a position within the host cell nucleic acid in which the polynucleotide is not normally found. An inserted polynucleotide may be introduced into the cell in a stable or transient manner, so as to produce a ribonucleic acid (RNA) molecule and/or a polypeptide molecule. The polynucleotide may comprise a nucleic acid sequence which is identical or partially homologous to an endogenous nucleic acid sequence of the cell. In certain embodiments, the polynucleotide is inserted into the exon of a secretory protein. Inserted polynucleotides are inserted in a manner such that the one or more peptides or proteins it encodes are secreted.

As described herein the inserted polynucleotide can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide).

Examples of inserted polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The polynucleotide may include signaling biochemical pathway-associated genes and polynucleotides as listed in U.S. Provisional Patent Applications 61/736,527 and 61/748,427 both entitled SYSTEMS METHODS AND COMPOSITIONS FOR SEQUENCE MANIPULATION filed on Dec. 12, 2012 and Jan. 2, 2013, respectively, and International Patent Application No. PCT/US2013/074667, entitled DELIVERY, ENGINEERING AND OPTIMIZATION OF SYSTEMS, METHODS AND COMPOSITIONS FOR SEQUENCE MANIPULATION AND THERAPEUTIC APPLICATIONS, filed Dec. 12, 2013, the contents of all of which are herein incorporated by reference in their entirety.

Examples of polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. In the context of disease associated genes, it is preferably a gene that becomes expressed at an abnormally low level, where the altered expression correlates with the occurrence and/or progression of the disease. The transcribed or translated products may be known or unknown, and may be at a normal or abnormal level.

The polynucleotides may encode one or more proteins, including those described herein. In some cases, the polynucleotides may encode fragments of the proteins, e.g., functional fragments of the proteins. The term “functional fragment” means that the sequence of the polypeptide may include less amino-acid than the original sequence but still enough amino-acids to confer the enzymatic activity of the full-length protein. A “functional fragment” may substantially retains at least one biological activity normally associated with that protein. In some examples, a functional fragment substantially retains all of the activities possessed by the full-length protein. By “substantially retains” biological activity, it is meant that the fragment retains at least 20%, at least 30%, at least 40%, at least 50%, at least 60%, at least 75%, at least 85%, at least 90%, at least 95%, at least 97%, at least 98%, or at least 99%, of the biological activity of the full-length protein. Small peptides from plant/animal origins with similar biological activity as a protein or functional fragment are also contemplated for use herein.

An engineered cell herein may comprise one or more inserted polynucleotides. For example, an engineered cell may comprise 1, 2, 3, 4, 5, 6, 7, 8, 9, 10, 11, 12, 13, 14, 15, 16, 17, 18, 19, 20 or more polynucleotides. An engineered cell may comprise polynucleotides encoding one or more proteins. For example, an engineered cell may comprise polynucleotides encoding 1, 2, 3, 4, 5, 6, 7, 8, 9, 10, 11, 12, 13, 14, 15, 16, 17, 18, 19, 20 or more proteins.

The polynucleotide(s) may be inserted in one or more endogenous genes, which may include insertion according to methods as described elsewhere herein. In some cases, the polynucleotide(s) may be inserted in one endogenous gene. Alternatively, or additionally, the polynucleotide(s) may be inserted in multiple endogenous genes. In preferred embodiments, the endogenous gene encodes for a secretory protein.

### Secretory Proteins

In some example embodiments, one or more of the polynucleotides may be inserted into a secretory protein, e.g., a protein that can be secreted from the cell. In these cases, the protein encoded by the polynucleotides may be secreted and function outside the cell, e.g., by affecting on cells and tissues surrounding the engineered cell. A secretory is a protein that is actively transported out of the cell, for example, the protein, whether it be endocrine or exocrine, is secreted by a cell.

Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., α1-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some aspects, the secretory protein is glucagon-like peptide-1 (GLP-1), glucagon, betatrophin, pancreatic amylase, pancreatic lipase, carboxypeptidase, secretin, CCK, a PPAR (e.g. PPAR-alpha, PPAR-gamma, PPAR-delta or a precursor thereof (e.g. preprotein or preproprotein). In aspects, the secretory protein is fibronectin, a clotting factor protein (e.g. Factor VII, VIII, IX, etc.), α2-macroglobulin, α1-antitrypsin, antithrombin III, protein S, protein C, plasminogen, α2-antiplasmin, complement components (e.g. complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Proteins

In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4⁺, CD8⁺, effector Th, memory Th, regulatory Th, CD4⁺/CD8⁺ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins may be immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-γ, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

The exogenous polynucleotides may encode one or more anti-fibrotic proteins. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR), or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration, Transplant Survival and/or Functions

The polynucleotide encoding one or more proteins may be proteins that promote tissue regeneration, transplant survival and/or functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfa1, Mafa, Pax6, Pax4, Nkx6.1, Nkx2.2, PDGF, vglycin, placental lactogens (somatomammotropins, e.g. CSH1, CHS2), isoforms thereof, homologs thereof, and orthologs thereof. In certain embodiments, the protein promoting pancreatic B cell regeneration is a cytokine, myokine, and/or adipokine.

### Hormones

The exogenous polynucleotides may encode one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoidds. In a particular embodiment, the hormone is insulin or incretins such as exenatide, GLP-1.

### Neurohormones

In embodiments, the secreted peptide is a neurohormone, a hormone produced and released by neuroendocrine cells. Neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In embodiments, the secreted protein is an anti-microbial. In embodiments where the cell is human cell, human host defense antimicrobial peptides and proteins (AMPs) play a critical role in warding off invading microbial pathogens. In certain embodiments, the anti-microbial is α-defensin HD-6, HNP-1 and β-defensin hBD-3, lysozyme, cathelcidin LL-37, C-type lectin RegIIIalpha, for example. See, e.g. Wang, “Human Antimicrobial Peptide and Proteins” Pharma, May 2014, 7(5): 545-594, incorporated herein by reference.

### Interference RNA

The polynucleotides may encode one or more interference RNAs. Interference RNAs are RNA molecules capable of suppressing gene expressions. Example types of interference RNAs include small interfering RNA (siRNA), micro RNA (miRNA), and short hairpin RNA (shRNA).

In some cases, the exogenous polynucleotides encode siRNAs. Small interfering RNA (siRNA) molecules are capable of inhibiting target gene expression by interfering RNA. siRNAs may be chemically synthesized, or may be obtained by in vitro transcription, or may be synthesized in vivo in target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs the antiparallel strands that form siRNA are connected by a loop or hairpin region.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway.

TGFβ pathway genes may comprise one or more of ACVR1, ACVR1C, ACVR2A, ACVR2B, ACVRL1, AMH, AMHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDFS, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

### Anti-Fibrillating Polypeptides

The polynucleotide may encode an anti-fibrillating polypeptide. The anti-fibrillating polypeptide can be the secreted polypeptide. In some aspects the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some aspects, the anti-fibrillating agent is P4 (VITYF)(SEQ ID NO: 1), P5 (VVVVV) (SEQ ID NO: 2), KR7 (KPWWPRR) (SEQ ID NO: 3), NK9 (NIVNVSLVK) (SEQ ID NO: 4), iAb5p (Leu-Pro-Phe-Phe-Asp) (SEQ ID NO: 5), KLVF and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA)(SEQ ID NO: 6), RI-OR2-TAT, cyclo(17, 21)-(Lys17, Asp21)A_(1-28), SEN304, SEN1576, D3, R8-Aβ(25-35), human yD-crystallin (HGD), poly-lysine, heparin, poly-Asp, polyGl, poly-L-lysine, poly-L-glutamic acid, LVEALYL (SEQ ID NO: 7), RGFFYT (SEQ ID NO: 8), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Aβ peptide. In some aspects, the polynucleotide encodes a fibrillation resistant protein. In some aspects, the fibrillation resistant protein is a modified insulin, see e.g. U.S. Pat. No. 8,343,914.

### Protease Cleavage Sites

The polynucleotides may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein encoded by the exogenous polynucleotides.

Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1 (ULP-1) protease cleavage site. In certain examples, the protease cleavage site comprises Lys-Arg.

The engineered cell may comprise one or more types of proteins or interference RNA described herein. In some examples, the engineered cells comprise exogenous polynucleotides encoding one or more immunosuppressive proteins and one or more anti-fibrotic proteins. In some examples, the engineered cells comprise exogenous polynucleotides encoding one or more immunosuppressive proteins and one or more hormones. In some examples, the engineered cells comprise exogenous polynucleotides encoding one or more immunosuppressive proteins and one or more proteins that promote transplant survival and functions. In some examples, the engineered cells comprise exogenous polynucleotides encoding one or more immunosuppressive proteins, one or more anti-fibrotic proteins, and one or more hormones. In some examples, the engineered cells comprise exogenous polynucleotides encoding one or more immunosuppressive proteins, one or more anti-fibrotic proteins, and one or more proteins that promote transplant survival and functions. In some examples, the engineered cells comprise exogenous polynucleotides encoding one or more immunosuppressive proteins, one or more hormones, and one or more proteins that promote transplant survival and functions. In some examples, the engineered cells comprise exogenous polynucleotides encoding one or more immunosuppressive proteins, one or more hormones, one or more anti-fibrotic proteins, and one or more proteins that promote transplant survival and functions.

### Types of Cells

The engineered cells may be any types of cells suitable for cell therapy. Engineered cells, cell lines, tissues and organoids from the engineered cells are also envisioned for use in the methods and systems detailed. The engineered cells may be generated by engineering cultured cells. In certain cases, the engineered cells may be primary cells, e.g., they are generated by engineering primary cells, e.g., cells isolated from a subject. As mentioned above, secretory pathways have been shown conserved from yeast to mammals, and both conventional and unconventional protein secretion pathways have been demonstrated in plants. Accordingly, the cells may be yeast cells, animal cells, plant cells. In embodiments, the cell is a eukaryotic cell.

The engineered cells may be hormone-secreting cells. Examples of hormone-secreting cells include certain cells of the pituitary gland, the endometrium, the ovary and the pancreas, anterior pituitary cells such as somatotropes, lactotropes, thyrotropes, gonadotropes, and corticotropes, pituitary cells, magnocellular neurosecretory cells, gut and respiratory tract cells, thyroid gland cells (thyroid epithelial cells and parafollicular cells), parathyroid gland cells (parathyroid chief cells and oxyphil cells), adrenal gland cells (chromaffin cells), Leydig cells of testes, theca interna cells of ovarian follicle, corpus luteum cells of ruptured ovarian follicle (including granulosa lutein cells and theca lutein cells), Juxtaglomerular cells (renin secretion), macula densa cells of kidney, peripolar cells of kidney, and mesangial cells of kidney. In some embodiments, the engineered cells may be endocrine cells, including pancreatic cells. The cells may be intestinal cells, in particular, enteroendocrine cells. Enteroendocrine cells secrete incretin products that parallels the secretion of insulin following oral glucose load, including GLP-1 and GLP-2.

In some examples, the engineered cells are pancreatic cells, e.g., pancreatic islet cells. Examples of pancreatic cells include pancreatic α cells, pancreatic β cells, pancreatic A cells, and pancreatic F cells. In one example, the engineered cells are pancreatic β cells.

In some embodiments, the engineered cells may be stem cells. For example, the engineered cells may be generated by engineering stem cells. As used herein the term “stem cell” (“SC”) refers to cells that can self-renew and differentiate into multiple lineages. A stem cell is a developmentally pluripotent or multipotent cell.

In some cases, the stem cells are embryonic stem cells, e.g., pluripotent cells derived from the inner cell mass of a blastocyst (e.g., a 4- to 5-day-old human embryo), and have the ability to yield many or all of the cell types present in a mature animal.

In some cases, the stem cells are adult stem cells. Examples of adult stem cells include hematopoietic stem cells, neural stem cells, mesenchymal stem cells, and bone marrow stromal cells. These stem cells have demonstrated the ability to differentiate into a variety of cell types, i.e. multipotent, including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

In some cases, the stem cells may be induced pluripotent stem cells. The term “induced pluripotent stem cells” (“iPSCs”) refers to a stem cell induced from a somatic cell, e.g., a differentiated somatic cell, and that has a higher potency than said somatic cell. iPS cells are capable of self-renewal and differentiation into mature cells.

In some embodiments, the engineered cells may be cells differentiated from stem cells. In some cases, the cells may be differentiated from stem cells in vitro. For examples, the cells may be pancreatic β cells differentiated from stem cells using methods described in WO2016100921A1, WO2015002724A2, US2019001703, WO2019018818. The term “in vitro” generally denotes outside, or external to, a body, e.g., an animal or human body. The terms “culturing” or “cell culture” are common in the art and broadly refer to maintenance of cells and potentially expansion (proliferation, propagation) of cells in vitro.

The engineered cells may be mammalian cells. For example, the cells may be cells of non-human mammals include, but are not limited to, non-human primates, rodents, bovines, ovines, equines, dogs, cats, goats, sheep, dolphins, bats, rabbits, and marsupial. In some examples, the cells may be human cells.

### Pharmaceutical Compositions

Pharmaceutical compositions or vaccines are also contemplated within the scope of the disclosure. The pharmaceutical compositions may comprise one or more the engineered cells described herein.

A “pharmaceutical composition” refers to a composition that usually contains an excipient, such as a pharmaceutically acceptable carrier that is conventional in the art and that is suitable for administration to cells or to a subject. Pharmaceutically acceptable as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8(2000), Wang W. “Lyophilization and development of solid protein pharmaceuticals.” Int. J. Pharm. 203 (1-2):1-60 (2000), Charman, W N “Lipids, lipophilic drugs, and oral drug delivery-some emerging concepts.” J Pharm Sci. 89(8):967-78(2000), Powell et al. “Compendium of excipients for parenteral formulations” PDAJ Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The pharmaceutical formulation may comprise a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range of a drug lie within the range known in the art for a particular drug or biologic. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

In certain embodiments, the pharmaceutical formulation may be topically administered to mucosa, such as the oropharynx, nasal cavity, respiratory tract, gastrointestinal tract, eye such as the conjunctival mucosa, vagina, urogenital mucosa, or for dermal application. In certain embodiments, the pharmaceutical formulation are administered to the nasal, bronchial or pulmonary mucosa. In order to obtain optimal delivery of the pharmaceutical formulation to the pulmonary cavity in particular, it may be advantageous to add a surfactant such as a phosphoglyceride, e.g. phosphatidylcholine, and/or a hydrophilic or hydrophobic complex of a positively or negatively charged excipient.

Other excipients suitable for pharmaceutical compositions intended for delivery of the pharmaceutical formulation to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the pharmaceutical formulation s of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivogene transfer techniques include transduction with viral (typically lentivirus, adeno associated virus (AAV) and adenovirus) vectors.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propyleneglycol or polyethylene glycol may be included. The composition may include one or more cell protective molecules, cell regenerative molecules, growth factors, anti-apoptotic factors or factors that regulate gene expression in the cells. Such substances may render the cells independent of their environment. Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating the support with a liquid suspension containing the cells. The impregnated supports obtained in this way can be implanted in a human subject. Such impregnated supports can also be re-cultured by immersing them in a liquid culture medium, prior to being finally implanted. The three-dimensional solid support needs to be biocompatible so as to enable it to be implanted in a human. It may be biodegradable or non-biodegradable.

The cells or cell populations can be administered in a manner that permits them to survive, grow, propagate and/or differentiate towards desired cell types (e.g. differentiation) or cell states. The cells or cell populations may be grafted to or may migrate to and engraft within the intended organ. In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

The pharmaceutical composition may further comprise a scaffold or matrix that can be used for transplanting the cells to a subject. A scaffold can provide a structure for the cell cluster to adhere to. The cell cluster can be transplanted to a subject with the scaffold. The scaffold can be biodegradable. In some cases, a scaffold comprises a biodegradable polymer. The biodegradable polymer can be a synthetic polymer, such as poly(lactide) (PLA), poly(glycolic acid) (PGA), poly(lactide-co-glycolide) (PLGA), and other polyhydroxyacids, poly(caprolactone), poly carbonates, polyamides, polyanhydrides, polyphosphazene, polyamino acids, polyortho esters, polyacetals, polycyanoacrylates, and biodegradable polyurethanes. The biodegradable polymer can also be a natural polymer, such as albumin, collagen, fibrin, polyamino acids, prolamines, and polysaccharides (e.g., alginate, heparin, and other naturally occurring biodegradable polymers of sugar units). Alternatively, the scaffold can be non-biodegradable. For example, a scaffold can comprise a non-biodegradable polymer, such as polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, and polyethylene oxide.

### Methods of Delivering Nucleic Acids to Engineer the Cells

In certain example embodiments, the invention further includes a method of delivering nucleic acids to engineer the cell. The method may comprise delivering any of the nucleic acids or vectors described herein to a first population of donor cells.

There are a variety of techniques available for introducing nucleic acids into viable cells. The techniques vary depending upon whether the nucleic acid is transferred into cultured cells in vitro, ex vivo, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

### Methods of Making Engineered Cells

The present disclosure also provides methods of making the engineered cells described herein. In general, the methods may include delivering the one or more exogenous polynucleotides to a cell. The methods may include inserting the one or more exogenous polynucleotides into the genome of a cell. The insertion may be performed using a gene editing approach. The composition may comprise a gene editing system, such as a CRISPR-Cas system, a Zinc finger nuclease system, a Transcription Activator-like Effector nuclease (TALEN) system, or a meganuclease system. The methods may include obtaining a cell from an organism and editing the cell to insert one or more polynucleotides. In preferred embodiments, the insertion is in-frame with the secretory protein. In certain embodiments, the insertion is in the exon coding portion of the secretory protein.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

A CRISPR-Cas system may comprise a Cas protein. A Cas protein may be Cas9, Cas 12 (e.g., Cas12a, Cas12b, Cas12c, Cas12d, etc.), Cas13 (e.g., Cas13a, Cas13b (such as Cas13b-t1, Cas13b-t2, Cas13b-t3), Cas13c, Cas13d, etc.), CasX, or CasY.

Additional Cas proteins for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

In some cases, the Cas protein is Cas9 or a variant thereof. The Cas9 gene is found in several diverse bacterial genomes, typically in the same locus with cas1, cas2, and cas4 genes and a CRISPR cassette Furthermore, the Cas9 protein contains a readily identifiable C-terminal region that is homologous to the transposon ORF-B and includes an active RuvC-like nuclease, an arginine-rich region.

In particular embodiments, the effector protein is a Cas9 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, or Corynebacterium.

In particular embodiments, the effector protein is a Cas9 effector protein from an organism from a genus comprising Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cas9 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii. In particular embodiments, the effector protein is a Cas9 effector protein from an organism from Streptococcus pyogenes, Staphylococcus aureus, or Streptococcus thermophilus Cas9.

The effector protein may comprise a chimeric effector protein comprising a first fragment from a first effector protein (e.g., a Cas9) ortholog and a second fragment from a second effector (e.g., a Cas9) protein ortholog, and wherein the first and second effector protein orthologs are different. At least one of the first and second effector protein (e.g., a Cas9) orthologs may comprise an effector protein (e.g., a Cas9) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cas9 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cas9 of S. mutans, S. agalactiae, S. equisimilis, S. sanguinis, S. pneumonia; C. jejuni, C. coli; N. salsuginis, N tergarcus; S. auricularis, S. carnosus; N meningitides, N gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In a more preferred embodiment, the Cas9 is derived from a bacterial species selected from Streptococcus pyogenes, Staphylococcus aureus, or Streptococcus thermophilus Cas9. In certain embodiments, the Cas9p is derived from a bacterial species selected from Francisella tularensis 1, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

The nucleic acid-targeting system may be derived advantageously from a Type VI CRISPR system. In some embodiments, one or more elements of a nucleic acid-targeting system is derived from a particular organism comprising an endogenous RNA-targeting system. In particular embodiments, the Type VI RNA-targeting Cas enzyme is C2c2. In an embodiment of the invention, there is provided a effector protein which comprises an amino acid sequence having at least 80% sequence homology to the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2.

In particular embodiments, the homologue or orthologue of Cas9 as referred to herein has a sequence homology or identity of at least 80%, more preferably at least 85%, even more preferably at least 90%, such as for instance at least 95% with Cas9. In further embodiments, the homologue or orthologue of Cas9 as referred to herein has a sequence identity of at least 80%, more preferably at least 85%, even more preferably at least 90%, such as for instance at least 95% with the wild type Cas9. Where the Cas9 has one or more mutations (mutated), the homologue or orthologue of said Cas9 as referred to herein has a sequence identity of at least 80%, more preferably at least 85%, even more preferably at least 90%, such as for instance at least 95% with the mutated Cas9.

In some embodiments, the Cas9 protein may be an ortholog of an organism of a genus which includes, but is not limited to Streptococcus sp. or Staphylococcus sp.; in particular embodiments, Cas9 protein may be an ortholog of an organism of a species which includes, but is not limited to Streptococcus pyogenes, Staphylococcus aureus, or Streptococcus thermophilus Cas9. In particular embodiments, the homologue or orthologue of Cas9p as referred to herein has a sequence homology or identity of at least 80%, more preferably at least 85%, even more preferably at least 90%, such as for instance at least 95% with one or more of the Cas9 sequences disclosed herein. In further embodiments, the homologue or orthologue of Cas9 as referred to herein has a sequence identity of at least 80%, more preferably at least 85%, even more preferably at least 90%, such as for instance at least 95% with the wild type SpCas9, SaCas9 or StCas9.

In particular embodiments, the Cas9 protein of the invention has a sequence homology or identity of at least 60%, more particularly at least 70, such as at least 80%, more preferably at least 85%, even more preferably at least 90%, such as for instance at least 95% with SpCas9, SaCas9 or StCas9. In further embodiments, the Cas9 protein as referred to herein has a sequence identity of at least 60%, such as at least 70%, more particularly at least 80%, more preferably at least 85%, even more preferably at least 90%, such as for instance at least 95% with the wild type SpCas9, SaCas9 or StCas9. The skilled person will understand that this includes truncated forms of the Cas9 protein whereby the sequence identity is determined over the length of the truncated form.

In an embodiment of the invention, the effector protein comprises at least one HEPN domain, including but not limited to HEPN domains described herein, HEPN domains known in the art, and domains recognized to be HEPN domains by comparison to consensus sequences and motifs.

In some cases, the CRISPR-Cas protein is Cas9 or a modified form thereof. In some examples, Cas9 may be wildtype Cas9 including any naturally-occurring bacterial Cas9. Cas9 orthologs typically share the general organization of 3-4 RuvC domains and a HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Similarly, the conserved His and Asn residues in the HNH domains are mutated to Alanine to convert Cas9 into a non-complementary-strand nicking enzyme. In some embodiments, both sets of mutations may be made, to convert Cas9 into a non-cutting enzyme. Accordingly, the Cas enzyme can be wildtype Cas9 including any naturally-occurring bacterial Cas9. The CRISPR, Cas or Cas9 enzyme can be codon optimized for HSC, or a modified version, including any chimaeras, mutants, homologs or orthologs. In an additional aspect of the disclosure, a Cas9 enzyme may comprise one or more mutations and may be used as a generic DNA binding protein with or without fusion to a functional domain. The mutations may be artificially introduced mutations or gain- or loss-of-function mutations. The mutations may include but are not limited to mutations in one of the catalytic domains (D10 and H840) in the RuvC and HNH catalytic domains, respectively. Further mutations have been characterized. In one aspect of the disclosure, the transcriptional activation domain may be VP64. In other aspects of the disclosure, the transcriptional repressor domain may be KRAB or SID4X. Other aspects of the disclosure relate to the mutated Cas 9 enzyme being fused to domains which include but are not limited to a nuclease, a transcriptional activator, repressor, a recombinase, a transposase, a histone remodeler, a demethylase, a DNA methyltransferase, a cryptochrome, a light inducible/controllable domain or a chemically inducible/controllable domain. The disclosure can involve sgRNAs or tracrRNAs or guide or chimeric guide sequences that allow for enhancing performance of these RNAs in cells. The CRISPR enzyme can be a type I or III CRISPR enzyme, e.g., a type II CRISPR enzyme. This type II CRISPR enzyme may be any Cas enzyme. A preferred Cas enzyme may be identified as Cas9 as this can refer to the general class of enzymes that share homology to the biggest nuclease with multiple nuclease domains from the type II CRISPR system. Ins some cases, the Cas9 enzyme is from, or is derived from, spCas9 or saCas9. By derived, Applicants mean that the derived enzyme is largely based, in the sense of having a high degree of sequence homology with, a wildtype enzyme, but that it has been mutated (modified) in some way as described herein.

One or more residues in Cas9 may be mutated to cysteines, e.g., for conjugation with functionalizing molecules herein. The residues may be on solvent-exposed loops of various Cas9 domains, and polar residues may be mutated to minimize potential structure disruption. In some examples, one or more of the following mutations are introduced to Cas9: M1C, S204C, E532C, K558C, Q826C, E945C, E1026C, E1068C, S1116C, and E1207C.

In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is about no more than 25%, 10%, 5%, 1%, 0.1%, 0.01%, or less of the DNA cleavage activity of the non-mutated form of the enzyme; an example can be when the DNA cleavage activity of the mutated form is nil or negligible as compared with the non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools). In particular, any or all of the following mutations may be in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s may also be used. Particularly preferred are D10 and H840 in SpCas9. However, in other Cas9s, residues corresponding to SpCas9 D10 and H840 are also preferred. For instance, an N580 or N580A mutation in SaCas9. Orthologs of SpCas9 can be used in the practice of the disclosure. A Cas enzyme may be identified Cas9 as this can refer to the general class of enzymes that share homology to the biggest nuclease with multiple nuclease domains from the type II CRISPR system. In some cases, the Cas9 enzyme is from, or is derived from, spCas9 (S. pyogenes Cas9) or saCas9 (S. aureus Cas9). StCas9″ refers to wild type Cas9 from S. thermophilus, the protein sequence of which is given in the SwissProt database under accession number G3ECR1. Similarly, S pyogenes Cas9 or spCas9 is included in SwissProt under accession number Q99ZW2. By derived, Applicants mean that the derived enzyme is largely based, in the sense of having a high degree of sequence homology with, a wildtype enzyme, but that it has been mutated (modified) in some way as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure includes many more Cas9s from other species of microbes, such as SpCas9, SaCa9, St1Cas9 and so forth. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defence in bacteria and archaea, Mole Cell 2010, January 15; 37(1): 7. The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions, one can generate chimeric Cas9 proteins. And Cas9 may be used as a generic DNA binding protein.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53. short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters—especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Semi-Synthetic Genome Editor with Multifunctionality

In some cases, the Cas protein is engineered so it is attached with one or more functionalizing molecules, creating a Semi-synthetic Genome Editor with Multifunctionality (“SynGEM”), as described in PCT/US2018/057182, entitled “Novel Nucleic Acid Modifiers,” and filed Oct. 23, 2018, incorporated herein by reference. The functionalizing molecules may facilitate precise insertion of the exogenous polynucleotides to the genome of the engineered cell.

Molecules that can be attached to the Cas protein include donor oligonucleotides, inhibitors of non-homologous end joining (NHEJ), inhibitors of genotoxicity, and adapter oligonucleotides, and can include attachment and molecules as described and in PCT/US2018/057182 at [0835]-[0846], incorporated herein by reference.

In some embodiments, the Cas protein may be attached with one or more donor oligonucleotides that comprise the oligonucleotides to be inserted to the genome of the engineered cell. The donor oligonucleotides may be single-stranded oligodeoxynucleotides (ssODN). As used herein the term “ssODN” refers to a single-stranded donor oligonucleotide or a single stranded DNA oligonucleotide are used to direct gene repair after a double strand nick has been induced in a gene using both CRISPR/Cas9 and TALENs. Locating ssODNs close to the DNA break site would enhance the rate of precision genome editing due to the increased local concentration. The ssODN may acts as a template for homology directed repair of said double strand break. The ssODN may be used in place of targeting plasmids for short modifications within a defined locus without cloning. The ssODN may be used to achieve high HDR efficiencies. For example, it may contain flanking sequences of at least 40 bp on each side that are homologous to the target region, and can be oriented in either the sense or antisense direction relative to the target locus.

The Cas protein may be attached with one or more NHEJ inhibitors. In the absence of a repair template, the NHEJ process re-ligates DSBs, which may leave a scar in the form of indel mutations. This process can be harnessed to achieve gene knockouts, as indels occurring within a coding exon may lead to frameshift mutations and a premature stop codon. Multiple DSBs may also be exploited to mediate larger deletions in the genome. NHEJ may result in random insertions and deletions. Inhibition of NHEJ may cause the cell to repair the break via the homology-directed repair (HDR) mechanism. Homology directed repair is an alternate major DNA repair pathway to NHEJ. Although HDR typically occurs at lower frequencies than NHEJ, it may be harnessed to generate precise, defined modifications at a target locus in the presence of an exogenously introduced repair template. The repair template may be either in the form of double stranded DNA, designed similarly to conventional DNA targeting constructs with homology arms flanking the insertion sequence, or single-stranded DNA oligonucleotides (ssODNs). The latter provides an effective and simple method for making small edits in the genome, such as the introduction of single nucleotide mutations for probing causal genetic variations. Unlike NHEJ, HDR is generally active only in dividing cells and its efficiency varies depending on the cell type and state. HDR activators, such as RS1 can be discplayed on the genome editor as well.

Examples of NHEJ inhibitors include inhibitors of DNA-dependent protein kinase (DNA-PK) such as viridins, wortmannin, quercitins, and LY294002, and inhibitors of ATM as described in US20040002492, which is incorporated by reference herein in its entirety. In some examples, the NHEJ inhibitors include SCR7 analogs, adenovirus 4 E1B55K, E4orf6, KU inhibitors, including KU-0060648.

The Cas protein may be attached with one or more inhibitors of genotoxicity. In some cases, the inhibitors of genotoxicity are inhibitors of p53 and its pathway. Examples of p53 inhibitors include p53 inhibitors typified by pifithrin (PFT)-α and -β, which are disclosed in WO 00/44364, PFT-μ disclosed in Storm et al. (Nat. Chem. Biol. 2, 474 (2006)), analogue thereof and salts thereof (for example, acid addition salts such as hydrochlorides and hydrobromides, and the like) and the like, PFT-α and analogues thereof [2-(2-Imino-4,5,6,7-tetrahydrobenzothiazol-3-yl)-1-p-tolylethanone, HBr (product name: Pifithrin-α) and 1-(4-Nitrophenyl)-2-(4,5,6,7-tetrahydro-2-imino-3(2H)-benzothiazolyl)ethanone, HBr (product name: Pifithrin-α, p-Nitro)], PFT-β and analogues thereof [2-(4-Methylphenyl)imidazo[2,1-b]-5,6,7,8-tetrahydrobenzothiazole, HBr (product name: Pifithrin-α, Cyclic) and 2-(4-Nitrophenyl)imidazo[2,1-b]-5,6,7,8-tetrahydrobenzothiazole (product name: Pifithrin-α, p-Nitro, Cyclic)], and PFT-μ, [Phenylacetylenylsulfonamide (product name: Pifithrin-μ)] are commercially available from Merck.

The Cas protein may be attached with one or more adapters. Such adapters may be universal adapters that can be an anchoring point for any kind of functional molecules based on DNA hybridization. The functionalizing molecules may be attached by conjugation. The term “conjugate” is intended to indicate a heterogeneous molecule formed by the covalent attachment of the Cas protein to one or more conjugation moieties such as polymer molecules, lipophilic compounds, carbohydrate moieties or organic derivatizing agents. The term covalent attachment means that the polypeptide and the conjugation moiety are either directly covalently joined to one another, or else are indirectly covalently joined to one another through an intervening moiety or moieties, such as a bridge, spacer, or linkage moiety or moieties.

A conjugation moiety may have a reactive group comprising aldehyde, propionic aldehyde, butyl aldehyde, maleimide, or succinimide derivative (e.g., succinimidyl propionate, succinimidyl carboxymethyl, hydroxy succinimidyl and succinimidyl carbonate). A conjugation moiety may have two reaction groups at one or two ends. The reactive groups at the both ends of the conjugation moiety may be identical to or different from each other. For example, a conjugation moiety may have a maleimide group at one end, and a maleimide group, an aldehyde group or a propionic aldehyde group at the other end. When poly(ethylene glycol) is used as the conjugation moiety, a commercially available product may be used for preparing the protein conjugate of the invention, or the terminal hydroxy groups of the commercial PEG may be further converted to other reactive groups before the coupling reaction.

In some examples, the conjugation may be performed using a multiple orthogonal strategy, e.g., using cysteine-maleimide, sortase chemistry, and unnatural amino acids bearing groups with orthogonal reactivity to cysteine and sortase. Site-specific C-terminal and internal loop labeling of proteins using sortase-mediated reactions. Guimaraes, C. P.; Witte, M. D.; Theile, C. S.; Bozkurt, G.; Kundrat, L.; Blom, A. E.; Ploegh, H. L. Nat Protoc 2013, 8, 1787-99.PMC3943461; Site-specific N-terminal labeling of proteins using sortase-mediated reactions. Theile, C. S.; Witte, M. D.; Blom, A. E.; Kundrat, L.; Ploegh, H. L.; Guimaraes, C. P. Nat Protoc 2013, 8, 1800-7.PMC3941705; Sortase-mediated ligations for the site-specific modification of proteins. Schmohl, L.; Schwarzer, D. Curr Opin Chem Biol 2014, 22, 122-8

### Methods of Treatment

The methods may also comprise modulating secreting proteins to confer a particular benefit or advantage to the engineered cell or to an organism into which the cell is transplanted or in which the cell is present. In particular embodiments, the systems and methods as described herein can be used to confer desired traits on essentially any organism, in preferred embodiments, eukaryotes, in particular embodiments, any organism with a secretory pathway can be exploited according to the methods disclosed herein.

The present disclosure further provides methods for treating a disease with the engineered cells. Such methods may include administering one or more of the engineered cells or a pharmaceutical composition comprising such cells to a subject in need thereof. The one or more engineered cells may be administered at a pharmaceutically effective amount. The term “pharmaceutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

### Administration

In some embodiments, the methods include administering one or more engineered cells to a subject (e.g., a subject in need thereof). In some cases, the engineered cells may be administered to the site of intended benefit is preferred. In some examples, the engineered cells may be administered intravascularly. Other routes of administration may include intratracheal delivery, intraventricular delivery, intrathecal delivery, intraosseous delivery, pulmonary delivery, buccal delivery, aerosol delivery, inhalational delivery, oral delivery, intraarterial delivery, intracerebral delivery, intraintestinal delivery, intracardiac delivery, subcutaneous delivery, intramuscular delivery, intraorbital delivery, intracapsular delivery, intraspinal delivery, intraperitoneal delivery, intrasternal delivery, intravesical delivery, intralymphatic delivery, intracavital delivery, vaginal delivery, rectal delivery, transurethral delivery, intradermal delivery, intraocular delivery, aural delivery, intramammary delivery, orthotopic delivery, intratracheal delivery, intralesional delivery, percutaneous delivery, endoscopical delivery, transmucosal delivery, sublingual delivery, and direct application on body surfaces (e.g., directly onto skin surface).

In some examples, the engineered cells are administered to the brain, e.g., intraventricularly. For example, the engineered cells may be administered to the cerebrospinal fluid (CSF) of a subject. In certain examples, the engineered cells may be administered to cerebral ventricle space. In some examples, the cells may be administered by lateral cerebro ventricular injection into the brain of a subject. The injection can be made, for example, through a burr hole made in the subject's skull. In certain examples, the cells and/or other pharmaceutical formulation may be administered through a surgically inserted shunt into the cerebral ventricle of a subject. For example, the injection may be made into the lateral ventricles, which are larger, even though injection into the third and fourth smaller ventricles can also be made.

In some examples, the engineered cells are administered intrathecally, e.g., to the spinal canal, the subarachnoid space, the lumbar area, and/or the cisterna magna. Intrathecal administration may include delivering the cells directly into the cerebrospinal fluid of a subject, by techniques including lateral cerebroventricular injection through a burrhole or cistemal or lumbar puncture or the like. The term “lumbar region” is intended to include the area between the third and fourth lumbar (lower back) vertebrae and, more inclusively, the L2-S1 region of the spine. The term “cisterna magna” is intended to include access to the space around and below the cerebellum via the opening between the skull and the top of the spine. The term “cerebral ventricle” is intended to include the cavities in the brain that are continuous with the central canal of the spinal cord.

In some embodiments, the cells may be placed in a delivery device which facilitates introduction by injection or implantation into the subjects. Examples of such delivery devices include tubes, e.g., catheters, endoscopic delivery devices, infusion pumps, or reservoir and catheters for intraventricular injection. The tubes may additionally have a needle.

The cells may be prepared for delivery in a variety of different forms. For example, the cells may be suspended in a solution or gel or embedded in a support matrix when contained in such a delivery device. The cells may be mixed with a pharmaceutically acceptable carrier or diluent in which the cells of the disclosure remain viable. Pharmaceutically acceptable carriers and diluents include saline, aqueous buffer solutions, solvents and/or dispersion media. The solution may be sterile and fluid. The solution may be stable under the conditions of manufacture and storage and preserved against the contaminating action of microorganisms such as bacteria and fungi through the use of, for example, parabens, chlorobutanol, phenol, ascorbic acid, thimerosal, and the like. Solutions may be prepared by incorporating cells as described herein in a pharmaceutically acceptable carrier or diluent and, as required, other ingredients, followed by filtered sterilization.

In some embodiments, the cells are introduced into the subject as part of a cell aggregate (e.g., a pancreatic islet), tissue, or organ, e.g., as part of an organ transplant method.

### Dosage

The cells may be introduced to the subject at a suitable dose. In some cases, from 4×10⁵ to 9×10⁶ cells per kilogram body weight of the subject. For example, from 4×10⁵ to 2×10⁶, from 1×10⁶ to 3×10⁶, from 2×10⁶ to 4×10⁶, from 3×10⁶ to 5×10⁶, from 4×10⁶ to 6×10⁶, from 5×10⁶ to 7×10⁶, from 6×10⁶ to 8×10⁶, or from 7×10⁶ to 9×10⁶ cells per kilogram body weight of the subject.

### Conditions and Diseases

The compositions and methods herein may be used in cell therapy for treating conditions and diseases. In some cases, the disease is diabetes, including, but not limited to, type I diabetes, type II diabetes, type 1.5 diabetes, prediabetes, cystic fibrosis-related diabetes, surgical diabetes, gestational diabetes, and mitochondrial diabetes. The disease may also be a diabetes complication, including heart and blood vessel diseases, diabetic nephropathy, diabetic neuropathy, diabetic retinopathy, foot damages, and hearing damages.

Subjects with diseases treated with immunotherapy drugs may benefit from treatment by the present methods and compositions. Diseases that can be treated by the present methods and compositions include cancer and other diseases that may be treated with immunotherapy. In particular, treatment with checkpoint inhibitors can trigger diabetes, with an estimation of about 1% of patients receiving immunotherapy drugs experiencing diabetes. Elie Dolgin, Stat News, “A Lifesaver with a Catch Powerful New Cancer Drugs Can Trigger Diabetes—and No One Knows Why,” May 29, 2019.

Diseases that can be treated by the present methods and compositions include those that can be treated by tissue regeneration or reconstitution, by protein replacement, or by coagulation factors. Such diseases include diseases associated with defective biological processes such as cardiac ischemia, osteoporosis, chronic wounds, diabetes, neural degenerative diseases, neural injuries, bone or cartilage injuries, ablated bone marrow, anemia, liver diseases, hair growth, teeth growth, retinal disease or injuries, ear diseases or injury, muscle degeneration or injury, plastic surgery. In addition, the treatment methods may be applied to cosmetic therapies including, filling of skin wrinkles, supporting organs, supporting surgical procedures, treating burns, and treating wounds, for example.

Diseases that can be treated by the present methods and compositions include autoimmune diseases. Autoimmune diseases include systemic lupus erythematosis, rheumatoid arthritis, Sjogren's syndrome, Reiter's syndrome, systemic sclerosis, polyarteritis nodosa, multiple sclerosis, juvenile oligoarthritis, collagen-induced arthritis, experimental autoimmune encephalomyelitis (EAE), inflammatory bowel disease (e.g. Crohn's disease, ulcerative colitis), autoimmune gastric atrophy, pemphigus vulgaris, psoriasis, vitiligo, type I diabetes, myasthenia gravis, Grave's disease, Hashimoto's thyroiditis, sclerosing sialadenitis, sclerosing cholangitis, Addison's disease, scleroderma, polymyositis, dermatomyositis, pernicious anemia, sympathetic ophthalmitis, and the like.

Diseases that can be treated by the present methods and compositions include genetic blood disorder, where exogenous stem cells of a normal phenotype are transplanted into the patient. Such diseases include, without limitation, the treatment of anemias caused by defective hemoglobin synthesis (hemoglobinopathies). The stem cells may be allogeneic stem cells of a normal phenotype, or may be autologous cells that have been genetically engineered to delete undesirable genetic sequences, and/or to introduce genetic sequences that correct the genetic defect. Sickle cell diseases include HbS Disease; drepanocytic anemia; meniscocytosis. Chronic hemolytic anemia occurring almost exclusively in blacks and characterized by sickle-shaped RBCs caused by homozygous inheritance of Hb S. Homozygotes have sickle cell anemia; heterozygotes are not anemic, but the sickling trait (sicklemia) can be demonstrated in vitro. In Hb S, valine is substituted for glutamic acid in the sixth amino acid of the beta chain. Deoxy-Hb S is much less soluble than deoxy-Hb A; it forms a semisolid gel of rodlike tactoids that cause RBCs to sickle at sites of low PO2. Distorted, inflexible RBCs adhere to vascular endothelium and plug small arterioles and capillaries, which leads to occlusion and infarction. Because sickled RBCs are too fragile to withstand the mechanical trauma of circulation, hemolysis occurs after they enter the circulation. In homozygotes, clinical manifestations are caused by anemia and vaso-occlusive events resulting in tissue ischemia and infarction. Growth and development are impaired, and susceptibility to infection increases. Anemia is usually severe but varies highly among patients. Anemia may be exacerbated in children by acute sequestration of sickled cells in the spleen. Thalassemias are a group of chronic, inherited, microcytic anemias characterized by defective Hb synthesis and ineffective erythropoiesis, particularly common in persons of Mediterranean, African, and Southeast Asian ancestry. Thalassemia is among the most common inherited hemolytic disorders. It results from unbalanced Hb synthesis caused by decreased production of at least one globin polypeptide chain (β, α, γ, δ). Aplastic anemia results from a loss of RBC precursors, either from a defect in stem cell pool or an injury to the microenvironment that supports the marrow, and often with borderline high MCV values. The term aplastic anemia commonly implies a panhypoplasia of the marrow with associated leukopenia and thrombocytopenia.

### Models of Genetic and Epigenetic Conditions

A method of the invention may be used to create a organism or cell that may be used to model and/or study genetic or epigenetic conditions of interest, such as a through a model of mutations of interest or a disease model.

As used herein, “disease” refers to a disease, disorder, or indication in a plant. For example, a method of the invention may be used to create an plant or cell that comprises a modification to include one or more polynucleotides associated with a lack of disease, or a plant or cell in which the expression of one or more nucleic acid sequences associated with a disease are altered. Such a nucleic acid sequence may encode a disease associated control sequence. Thus, the invention provides a plant, produced by the present methods, or a progeny thereof. The progeny may be a clone of the produced plant or may result from sexual reproduction by crossing with other individuals of the same species to introgress further desirable traits into their offspring. The cell may be in vivo or ex vivo in the cases of multicellular organisms, particularly plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

### Disease Model

In some methods, the disease model can be used to study the effects of mutations on the organism, for example, plant, animal, or cell and development and/or progression of the disease using measures commonly used in the study of the disease. Alternatively, such a disease model is useful for studying the effect of an active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a secretory protein with a polynucleotide such that protein from the inserted polynucleotide is produced and, as a result, the organism or cell has an altered response. Accordingly, in some methods, a genetically modified cell or organism may be compared with cell or organism predisposed to development of the disease such that the effect of the gene therapy event may be assessed.

A cell model, tissue model, or organism model can be constructed in combination with the method of the invention for screening a cellular function change. A genetically diverse mouse model can be utilized for screening. See, French et al. 2014. Diversity Outbred mice identify population based exposure thresholds and genetic factors that influence benzene-induced genotoxicity. Environ Health Perspect: doi:10.1289/ehp.1408202. Such a model may be used to study the effects of the protein expression of the invention on a cellular function of interest. For example, a cellular function model may be used to study the effect of a modified target sequence on intracellular signaling or extracellular signaling. Alternatively, a cellular function model may be used to study the effects of a modified sequence on sensory perception. In some such models, one or more target sequences associated with a signaling biochemical pathway in the model are modified.

An altered expression of one or more peptides or proteins associated with a signaling biochemical pathway can be determined by assaying for a difference in the levels of the corresponding protein expressions between the test model cell and a control cell, when they are contacted with a candidate agent. Alternatively, the differential expression associated with a signaling biochemical pathway is determined by detecting a difference in the level of the encoded polypeptide.

### Assays

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent:protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent: polypeptide complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Antiphosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2a). Alternatively, these antibodies can be generated using conventional polyclonal or monoclonal antibody technologies by immunizing a host animal or an antibody-producing cell with a target protein that exhibits the desired post-translational modification.

In practicing the subject method, it may be desirable to discern the expression pattern of aprotein associated with a signaling biochemical pathway in different bodily tissue, in different cell types, and/or in different subcellular structures. These studies can be performed with the use of tissue-specific, cell-specific or subcellular structure specific antibodies capable of binding to protein markers that are preferentially expressed in certain tissues, cell types, or subcellular structures.

An altered expression of a gene associated with a signaling biochemical pathway can also be determined by examining a change in activity of the gene product relative to a control cell. The assay for an agent-induced change in the activity of a protein associated with a signaling biochemical pathway will dependent on the biological activity and/or the signal transduction pathway that is under investigation. For example, where the protein is a kinase, a change in its ability to phosphorylate the downstream substrate(s) can be determined by a variety of assays known in the art. Representative assays include but are not limited to immunoblotting and immunoprecipitation with antibodies such as anti-phosphotyrosine antibodies that recognize phosphorylated proteins. In addition, kinase activity can be detected by high throughput chemiluminescent assays such as AlphaScreen™ (available from Perkin Elmer) and eTag™ assay (Chan-Hui, et al. (2003) Clinical Immunology 111: 162-174).

Where the protein associated with a signaling biochemical pathway is part of a signaling cascade leading to a fluctuation of intracellular pH condition, pH sensitive molecules such as fluorescent pH dyes can be used as the reporter molecules. In another example where the protein associated with a signaling biochemical pathway is an ion channel, fluctuations in membrane potential and/or intracellular ion concentration can be monitored. A number of commercial kits and high-throughput devices are particularly suited for a rapid and robust screening for modulators of ion channels. Representative instruments include FLIPR™ (Molecular Devices, Inc.) and VIPR (Aurora Biosciences). These instruments are capable of detecting reactions in over 1000 sample wells of a microplate simultaneously, and providing real-time measurement and functional data within a second or even a minisecond.

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

### Plant Cells

In one embodiment, the cell is a plant cell. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape) and plants used for experimental purposes (e.g., Arabidopsis). Thus, the methods and CRISPR-Cas systems can be used over a broad range of plants, such as for example, with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; the methods and CRISPR-Cas systems can be used with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

The RNA targeting CRISPR systems and methods of use described herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

The RNA targeting CRISPR systems and methods of use can also be used over a broad range of “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

A part of a plant, i.e., a “plant tissue” or “plant part” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized units, such as, for example, plant tissue, a plant organ, or a whole plant. The term “plant cell” as used herein also encompasses plant protoplasts.

A “protoplast” refers to a plant cell that has had its protective cell wall completely or partially removed using, for example, mechanical or enzymatic means resulting in an intact biochemical competent unit of living plant that can reform their cell wall, proliferate, regenerate and grow into a whole plant under proper growing conditions.

The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendents of any of these, such as cuttings or seed.

The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

The term “progeny”, such as the progeny of a transgenic plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

The term “plant promoter” as used herein is a promoter capable of initiating transcription in plant cells, whether or not its origin is a plant cell. Exemplary suitable plant promoters include, but are not limited to, those that are obtained from plants, plant viruses, and bacteria such as Agrobacterium or Rhizobium which comprise genes expressed in plant cells. Examples of plant promoters include, for example, the Cauliflower Mosaic Virus CaMV35S promoter and the maize ubiquitin gene Ubi promoter. Preferred promotors for the expression of a C2c2 effector protein include the rice actin promoter for monocots and the 35S promoter for dicots. The gRNA may preferably be under a rice U6 promoter in monocots or under a arabidopsis U6 promoter in dicots.

As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors may contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ, plasmids, yeast integrative plasmids, yeast replicative plasmids, shuttle vectors, and episomal plasmids.

### Plant Promoters

In order to ensure appropriate expression in a plant cell, the components of the CRISPR system described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. The present invention envisages methods for modifying RNA sequences and, as such, also envisages regulating expression of plant biomolecules. In particular embodiments of the present invention, it is thus advantageous to place one or more elements of the RNA targeting CRISPR system under the control of a promoter that can be regulated. “Regulated promoter” refers to promoters that direct gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In particular, placing one or more elements of the RNA targeting CRISPR system under the control of a promoter that directs expression of a gene in response to infection with a plant pathogen is envisaged. In particular embodiments, one or more of the RNA targeting CRISPR components are expressed under the control of a constitutive promoter, such as the cauliflower mosaic virus 35S promoter. Tissue-preferred promoters can be utilized to target enhanced expression in certain cell types within a particular plant tissue, for instance vascular cells in leaves or roots or in specific cells of the seed. Examples of particular promoters for use in the RNA targeting CRISPR system—are found in Kawamata et al., (1997) Plant Cell Physiol 38:792-803; Yamamoto et al., (1997) Plant J 12:255-65; Hire et al, (1992) Plant Mol Biol 20:207-18; Kuster et al, (1995) Plant Mol Biol 29:759-72; and Capana et al., (1994) Plant Mol Biol 25:681-91. Preferred promotors for the expression of a C2c2 effector protein include the rice actin promoter for monocots and the 35S promoter for dicots. The gRNA may preferably be under a rice U6 promoter in monocots or under a arabidopsis U6 promoter in dicots.

Examples of promoters that are inducible and that allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome), such as a Light Inducible Transcriptional Effector (LITE) that direct changes in transcriptional activity in a sequence-specific manner. The components of a light inducible system may include a RNA targeting CRISPR enzyme, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, which is hereby incorporated by reference in its entirety.

In particular embodiments, transient or inducible expression can be achieved by using, for example, chemical-regulated promoters, i.e. whereby the application of an exogenous chemical induces gene expression. Modulating of gene expression can also be obtained by a chemical-repressible promoter, where application of the chemical represses gene expression. Chemical-inducible promoters include, but are not limited to, the maize 1n2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

Translocation to and/or Expression in Specific Plant Organelles

The expression system may comprise elements for translocation to and/or expression in a specific plant organelle.

### Chloroplast Targeting

In particular embodiments, it is envisaged that the RNA targeting CRISPR system is used to specifically modify expression and/or translation of chloroplast genes or to ensure expression in the chloroplast. For this purpose, use is made of chloroplast transformation methods or compartimentalization of the RNA targeting CRISPR components to the chloroplast. For instance, the introduction of genetic modifications in the plastid genome can reduce biosafety issues such as gene flow through pollen.

Methods of chloroplast transformation are known in the art and include particle bombardment, PEG treatment, and microinjection. Additionally, methods involving the translocation of transformation cassettes from the nuclear genome to the plastid can be used as described in WO2010061186.

Alternatively, it is envisaged to target one or more of the RNA targeting CRISPR components to the plant chloroplast. This is achieved by incorporating in the expression construct a sequence encoding a chloroplast transit peptide (CTP) or plastid transit peptide, operably linked to the 5′ region of the sequence encoding the RNA targeting protein. The CTP is removed in a processing step during translocation into the chloroplast. Chloroplast targeting of expressed proteins is well known to the skilled artisan (see, for instance, Protein Transport into Chloroplasts, 2010, Annual Review of Plant Biology, Vol. 61: 157-180). In such embodiments, it is also desired to target the one or more guide RNAs to the plant chloroplast. Methods and constructs which can be used for translocating guide RNA into the chloroplast by means of a chloroplast localization sequence are described, for instance, in US 20040142476, incorporated herein by reference. Such variations of constructs can be incorporated into the expression systems of the invention to efficiently translocate the RNA targeting-guide RNA(s).

### Introduction of Polynucleotides in Algal Cells

Transgenic algae (or other plants such as rape) may be particularly useful in the production of vegetable oils or biofuels such as alcohols (especially methanol and ethanol) or other products. These may be engineered to express or overexpress high levels of oil or alcohols for use in the oil or biofuel industries.

### Introduction of Polynucleotides Encoding RNA Targeting Components in Yeast Cells

In particular embodiments, the invention relates to the use of the RNA targeting CRISPR system for RNA editing in yeast cells. Methods for transforming yeast cells which can be used to introduce polynucleotides encoding the RNA targeting CRISPR system components are well known to the artisan and are reviewed by Kawai et al., 2010, Bioeng Bugs. 2010 Nov.-Dec.; 1(6): 395-403). Non-limiting examples include transformation of yeast cells by lithium acetate treatment (which may further include carrier DNA and PEG treatment), bombardment or by electroporation.

### Transient Expression of RNA Targeting CRISP System Components in Plants and Plant Cells

In particular embodiments, it is envisaged that the guide RNA and/or RNA targeting gene are transiently expressed in the plant cell. In these embodiments, the RNA targeting CRISPR system can ensure modification of RNA target molecules only when both the guide RNA and the RNA targeting protein is present in a cell, such that gene expression can further be controlled. As the expression of the RNA targeting enzyme is transient, plants regenerated from such plant cells typically contain no foreign DNA. In particular embodiments, the RNA targeting enzyme is stably expressed by the plant cell and the guide sequence is transiently expressed.

In particularly preferred embodiments, the RNA targeting CRISPR system components can be introduced in the plant cells using a plant viral vector (Scholthof et al. 1996, Annu Rev Phytopathol. 1996; 34:299-323 and WO 2015189693 which describes the delivery of gRNA using a plant virus vector). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). For example, the viral vector may be a pWSRi vector as described in Golenberg et al., Plant Methods, 2009 or a BeYDV vector as described by Chen et al., Human Vaccines, 2011. In other particular embodiments, said viral vector is a vector from an RNA virus. For example, tobravirus (e.g., tobacco rattle virus (TRV), tobacco mosaic virus), potexvirus (e.g., potato virus X), or hordeivirus (e.g., barley stripe mosaic virus). The replicating genomes of plant viruses are non-integrative vectors, which is of interest in the context of avoiding the production of GMO plants.

In particular embodiments, the vector used for transient expression of RNA targeting CRISPR constructs is, for instance, a pEAQ vector, which is tailored for Agrobacterium-mediated transient expression (Sainsbury F. et al., Plant Biotechnol J. 2009 Sep.; 7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi:10.1038/srep14926).

In particular embodiments, double-stranded DNA fragments encoding the guide RNA and/or the RNA targeting gene can be transiently introduced into the plant cell. In such embodiments, the introduced double-stranded DNA fragments are provided in sufficient quantity to modify RNA molecule(s) in the cell but do not persist after a contemplated period of time has passed or after one or more cell divisions. Methods for direct DNA transfer in plants are known by the skilled artisan (see, for instance, Davey et al. Plant Mol Biol. 1989 Sep.; 13(3):273-85.)

In other embodiments, an RNA polynucleotide encoding the RNA targeting protein is introduced into the plant cell, which is then translated and processed by the host cell generating the protein in sufficient quantity to modify the RNA molecule(s) cell (in the presence of at least one guide RNA) but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122). Combinations of the different methods described above are also envisaged.

### Inserted Polynucleotide Envisaged for Plant, Algae or Fungal Applications

In preferred embodiments, the polynucleotide is a plant mRNA encoded by a resistance gene. The terms “resistance gene,” “disease resistance gene” and “R gene” are used interchangeably herein to denote a gene encoding a polypeptide capable of mediating or contributing to resistance to a specific plant pathogen. A “plant pathogen” is a disease-causing organism which attacks plants, a “disease” being any deviation from normal functioning of physiological processes of sufficient duration to cause disturbance or cessation of vitality. There are numerous types of pathogens which can target plants, including fungal pathogens, oomycetes, bacteria, viruses and viroids. The polypeptide encoded by the resistance gene may, for example, mediate resistance to a specific pathogen by triggering a defense response to said pathogen in a plant cell or plant tissue. A defense response is an active defensive reaction by a host, e.g. a plant, that stops or limits the growth and/or spread of the pathogen. The resulting resistance can be characterized by an absence or reduction in symptoms that would be present on inoculated plant tissue in the absence of such a response and/or by the pathogen being unable to complete its life cycle and/or to multiply or spread. A polypeptide encoded by a resistance gene may, for example, mediate the recognition of specific pathogen effectors, which are usually encoded by the pathogens avirulence (Avr) genes, either by directly binding thereto or by recognizing an alteration in a host protein that is caused by the pathogen. A plant carrying a specific resistance gene is, therefore, effectively protected from a pathogen carrying the corresponding avirulence gene. Resistance genes for different pathogens in different plants have been identified and are known in the art. A database providing an overview of resistance genes (R-genes) in plants can be found, for example at http://www.prgdb.org (see Sanseverino et al, 2010 Nucleic Acids Res).

### Use in Biofuel Production

The term “biofuel” as used herein is an alternative fuel made from plant and plant-derived resources. Renewable biofuels can be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. There are two types of biofuels: bioethanol and biodiesel. Bioethanol is mainly produced by the sugar fermentation process of cellulose (starch), which is mostly derived from maize and sugar cane. Biodiesel on the other hand is mainly produced from oil crops such as rapeseed, palm, and soybean. Biofuels are used mainly for transportation.

### Modifying Yeast for Biofuel Production

In particular embodiments, the RNA targeting enzyme provided herein is used for bioethanol production by recombinant micro-organisms. For instance, the methods and systems can be used to engineer micro-organisms, such as yeast, to generate biofuel or biopolymers from fermentable sugars and optionally to be able to degrade plant-derived lignocellulose derived from agricultural waste as a source of fermentable sugars. More particularly, the invention provides methods to insert one or more polynucleotides for increased expression and/or modify the expression of endogenous genes required for biofuel production. More particularly, the methods involve stimulating the expression in a micro-organism such as a yeast of one or more nucleotide sequence encoding enzymes involved in the conversion of pyruvate to ethanol or another product of interest. In particular embodiments, the methods ensure the increased expression of expression of one or more enzymes which allows the micro-organism to degrade cellulose, such as a cellulase.

### Modifying Algae and Plants for Production of Vegetable Oils or Biofuels

Transgenic algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries using the secretory approaches described herein.

U.S. Pat. No. 8,945,839 describes a method for engineering Micro-Algae (Chlamydomonas reinhardtii cells) species using Cas9. Using similar tools, the methods of the RNA targeting CRISPR system described herein can be applied on Chlamydomonas species and other algae. In particular embodiments, the RNA targeting effector protein and guide RNA are introduced in algae expressed using a vector that expresses the RNA targeting effector protein under the control of a constitutive promoter such as Hsp70A-Rbc S2 or Beta2-tubulin. Guide RNA will be delivered using a vector containing T7 promoter. Alternatively, in vitro transcribed guide RNA can be delivered to algae cells. Electroporation protocol follows standard recommended protocol from the GeneArt Chlamydomonas Engineering kit.

### Improved Plants

The present invention also provides plants and yeast cells obtainable and obtained by the methods provided herein. The improved plants obtained by the methods described herein may be useful in food or feed production through the modified expression of genes which, for instance ensure tolerance to plant pests, herbicides, drought, low or high temperatures, excessive water, etc.

The improved plants obtained by the methods described herein, especially crops and algae may be useful in food or feed production through expression of, for instance, higher protein, carbohydrate, nutrient or vitamin levels than would normally be seen in the wildtype. In this regard, improved plants, especially pulses and tubers are preferred.

Improved algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries.

The invention also provides for improved parts of a plant. Plant parts include, but are not limited to, leaves, stems, roots, tubers, seeds, endosperm, ovule, and pollen. Plant parts as envisaged herein may be viable, nonviable, regeneratable, and/or non-regeneratable.

It is also encompassed herein to provide plant cells and plants generated according to the methods of the invention. Gametes, seeds, embryos, either zygotic or somatic, progeny or hybrids of plants comprising the genetic modification, which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

In one aspect, the invention provides a kit comprising one or more of the components described hereinabove. In some embodiments, the kit comprises a vector system as described above and instructions for using the kit.

In an aspect, the invention provides a method of screening for gain of function (GOF) or loss of function (LOF) or for screening non-coding RNAs or potential regulatory regions (e.g. enhancers, repressors) comprising the cell line of as herein-discussed or cells of the model herein-discussed containing or expressing the RNA targeting enzyme and introducing a composition as herein-discussed into cells of the cell line or model, whereby the gRNA includes either an activator or a repressor, and monitoring for GOF or LOF respectively as to those cells as to which the introduced gRNA includes an activator or as to those cells as to which the introduced gRNA includes a repressor.

In an aspect, the invention provides a library of non-naturally occurring or engineered compositions, each comprising a RNA targeting CRISPR guide RNA (gRNA) comprising a guide sequence capable of hybridizing to a target RNA sequence of interest in a cell, an RNA targeting enzyme, wherein the RNA targeting enzyme comprises at least one mutation, such that the RNA targeting enzyme has no more than 5% of the nuclease activity of the RNA targeting enzyme not having the at least one mutation, wherein the gRNA is modified by the insertion of distinct RNA sequence(s) that bind to one or more adaptor proteins, and wherein the adaptor protein is associated with one or more functional domains, wherein the composition comprises one or more or two or more adaptor proteins, wherein the each protein is associated with one or more functional domains, and wherein the gRNAs comprise a genome wide library comprising a plurality of RNA targeting guide RNAs (gRNAs). In an aspect, the invention provides a library as herein-discussed, wherein the RNA targeting RNA targeting enzyme has a diminished nuclease activity of at least 97%, or 100% as compared with the RNA targeting enzyme not having the at least one mutation. In an aspect, the invention provides a library as herein-discussed, wherein the adaptor protein is a fusion protein comprising the functional domain. In an aspect, the invention provides a library as herein discussed, wherein the gRNA is not modified by the insertion of distinct RNA sequence(s) that bind to the one or two or more adaptor proteins. In an aspect, the invention provides a library as herein discussed, wherein the one or two or more functional domains are associated with the RNA targeting enzyme. In an aspect, the invention provides a library as herein discussed, wherein the cell population of cells is a population of eukaryotic cells. In an aspect, the invention provides a library as herein discussed, wherein the eukaryotic cell is a plant cell or a yeast cell.

In an aspect, the invention provides a library as herein discussed, wherein the targeting is of about 100 or more RNA sequences. In an aspect, the invention provides a library as herein discussed, wherein the targeting is of about 1000 or more RNA sequences. In an aspect, the invention provides a library as herein discussed, wherein the targeting is of about 20,000 or more sequences. In an aspect, the invention provides a library as herein discussed, wherein the targeting is of the entire transcriptome. In an aspect, the invention provides a library as herein discussed, wherein the targeting is of a panel of target sequences focused on a relevant or desirable pathway. In an aspect, the invention provides a library as herein discussed, wherein the pathway is an immune pathway. In an aspect, the invention provides a library as herein discussed, wherein the pathway is a cell division pathway.

In one aspect, the invention provides a method of generating a model eukaryotic cell comprising a gene with modified expression. In some embodiments, a disease gene is any gene associated with an increase in the risk of having or developing a disease. In some embodiments, the method comprises (a) introducing one or more vectors encoding the components of the system described herein above into a eukaryotic cell, and (b) allowing a CRISPR complex to bind to a target polynucleotide so as to modify expression of a gene, thereby generating a model eukaryotic cell comprising modified gene expression.

The present application also provides aspects and embodiments as set forth in the following numbered Statements:

1. An engineered cell comprising one or more polynucleotides encoding: one or more secreted proteins, or a functional fragment thereof, wherein the one or more secreted proteins or functional fragments thereof are inserted in one or more endogenous genes of the cell, the one or more endogenous genes encoding a secretory protein or peptide; an optional co-expressed protein that enhances the expression, stability or biological activity of the secreted protein.

2. The cell of Statement 1, wherein the secreted protein is an immunomodulatory protein, an anti-fibrotic, a hormone, anti-microbial or a protein or peptide that promotes tissue regeneration

3. The cell of Statement 2, wherein the secreted protein is an immunomodulatory protein and the co-expressed protein is an anti-fibrotic protein.

4. The cell of Statement 2, wherein the immunomodulatory protein is a cytokine.

5. The cell of Statement 2, wherein the immunomodulatory protein is IL-2, IL-4, IL-6, IL-10, IL-15, or IL-22.

6. The cell of Statement 2, wherein the secreted protein is an anti-fibrotic protein, optionally wherein the anti-fibrotic protein is a peroxisome proliferator-activated receptor.

7. The cell of Statement 2, wherein the secreted protein is a protein or peptide that promotes tissue regeneration, optionally Reg1A, Reg1B, Reg2, Reg3A, Reg3g, and/or Reg 4, or a combination thereof.

8. The cell of Statement 2, wherein the secreted protein is a hormone.

9. The cell of Statement 8, wherein the hormone is insulin.

10. The cell of Statement 8, wherein the hormone is a neuroendocrine hormone, optionally selected from thyrotropin-releasing hormone, corticotropin-releasing hormone, histamine, growth hormone-releasing hormone, somatostatin, gonadotropin-releasing hormone, serotonin, dopamine, neurotensin, oxytocin, vasopressin, epinephrine, and norepinephrine.

11. The cell of Statement 2, wherein the secreted protein is an anti-microbial, optionally wherein the anti-microbial is α-defensin HD-6, HNP-1 and β-defensin hBD-3

12. The cell of Statement 1, wherein the cell is a eukaryotic cell.

13. The cell of Statement 1, wherein the cell is a human cell.

14. The cell of Statement 1, wherein the cell is an cell or β cell.

15. The cell of Statement 1, wherein the cell is a pancreatic β cell.

16. The cell of Statement 15, wherein the pancreatic β cell is differentiated from a progenitor in vitro.

17. The cell of Statement 1, wherein the cell is a stem cell.

18. The cell of Statement 1, wherein the cell is a primary cell.

19. The cell of Statement 1, wherein the cell is a plant cell.

20. The cell of Statement 1, further comprising one or more polynucleotides encoding a protein promoting pancreatic β cell regeneration.

21. The cell of Statement 20, wherein the protein is a cytokine.

22. The cell of Statement 21, wherein the cytokine is IL-22.

23. The cell of Statement 20, wherein the protein upregulates one or more genes promoting pancreatic β cell regeneration.

24. The cell of Statement 23, wherein the one or more genes promoting pancreatic β cell regeneration is Reg1, Reg2, or a combination thereof.

25. The cell of Statement 1, further comprising one or more polynucleotides encoding interference RNA.

26. The cell of Statement 25, wherein the interference RNA is siRNA.

27. The cell of Statement 25, wherein the interference RNA suppresses TGF-β, colony-stimulating factor 1, or a combination thereof.

28. The cell of Statement 1, wherein the one or more inserted polynucleotides comprise one or more protease cleavage sites.

29. The cell of Statement 28, wherein the one or more inserted polynucleotides comprise a protease cleavage site on each end.

30. The cell of Statement 1, wherein the secretory protein or peptide is c-peptide.

31. A method of treating a disease comprising administering a cell of Statement 1 to a subject in need thereof.

32. The method of Statement 31, wherein the disease is diabetes.

33. A method of making the cell of Statement 1, comprising. obtaining the cell from an organism; editing the cell to insert one or more polynucleotides, wherein insertion is in-frame with the secretory protein.

34. The method of Statement 33, wherein the editing the cell is with a nucleic acid editing system.

35. The method of Statement 34, wherein the nucleic acid editing system is selected from a CRISPR nucleic acid editing system, zinc finger nuclease, TALEN, meganuclease, or a Semisynthetic Genome Editing Multifunctional system (Syngem).

36. The method of Statement 33, wherein the one or more polynucleotides are inserted in the exon portion of the secretory protein.

37. The method of Statement 33, wherein the editing is performed ex-vivo.

38. The method of Statement 33, further comprising the step of inserting the cell into an organism.

39. The method of Statement 38, wherein the organism is the same organism.

40. The method of Statement 38, wherein inserting the cell is by infusion.

41. The method of Statement 34, wherein the peptide is a c-peptide.

42. The method of Statement 41, wherein the editing the cell comprises using the CRISPR editing system comprising a gRNA that targets the c-peptide in the middle region of the c-peptide.

### EXAMPLES

### Example 1

This example shows exemplary methods to engineer islets with CRISPR-Cas9 based technology for long-term, self-mediated secretion of low-level immunosuppressive cytokines to abate chronic immune attacks and inhibit fibrosis formation. Applicants knock-in the genes for siRNA of TGF-β and colony-stimulating factor 1 (CSF1) to inhibit the TGF-β and CSF1R pathway to ensure long-term survival and functionality of encapsulated islets. Applicants first contend with the difficulties in precision genome editing and overcome the predisposition of non-homologous end joining (NHEJ) repair which can lead to the p53 apoptosis pathway. To achieve a system where delivery of anti-fibrotic agents can be finely tuned to safely reduce fibrosis, Applicants use a novel chemical conjugation approach, where concentrations of homologous repair-biasing molecules will be increased at the site of edit and non-homologous repair pathway will be locally inhibited to achieve high fidelity, nontoxic gene knock-in. With increased targetability to β-cells, Applicants knock-in the immunosuppressive and antifibrotic genes into islets to elicit a long-term and local regulatory effect to enhance graft survival.

Endow β-cells with immunomodulatory/anti-fibrotic properties using a chemically-enhanced Cas9. Applicants developed a chemical platform to enhance precision genome editing using Cas9 and were able to efficiently engineer INS1E cells to secrete IL-10. Applicants develop and optimize this chemically modified Cas9 in the context of human beta cells (islets or stem-cell-derived) to enable them to secrete immunomodulatory molecules.

Applicants develop first-in-class semi-synthetic, multifunctional genome editor by chemically-enhancing the functionality of Cas9 in the context of human beta cells. This is the first approach for genome editing in β-cells for immunosuppressive and antifibrotic properties. The locus of the c-peptide gene is of particular interest due to the high secretory profile of c-peptide, which enables the transcription and translation of inserted genes more likely. Current CRISPR-based technologies have to manage all aspects of the gene therapy process, including selection of genomic integration site, DNA cleavage, donor DNA delivery, and cell repair outcome. Demonstrated herein is an “all-in-one” approach by pioneering the development of a multifunctional Cas9 whose capacities are augmented using conjugated small molecules and donor DNAs. The conjugation uses a powerful array of cell-compatible chemical conjugation techniques that can enhance the endogenous function of a given protein. Applicants apply these powerful approaches toward the building of a suite of active Cas9 proteins capable of multivalent, orthogonal, and novel chemical conjugation. Current approaches toward increasing high knock-in involves global inhibition of NHEJ repair pathways across the cell, which requires high concentrations of exogenously supplied inhibitor that can lead to undesired toxicity and mutagenicity. Through chemical conjugation, Applicants develop strategies to locally enhance the concentration of cell repair-biasing molecules at the target site, leading to high fidelity and nontoxic repair.

Since the c-peptide is cleaved off during insulin processing and secreted, Applicants hypothesized that knocking in a desired gene into the c-peptide region of Insulin locus would enable the secretion of the inserted gene product. Indeed, a proinsulin-luciferase fusion construct, which has a G. luciferase in the middle of the c-peptide locus, expressed functional luciferase when stably integrated in INS1E cells. Here, the expression level of luciferase was directly proportional to that of insulin, and responded sensitively to external stimuli such as glucose concentration. Thus, inserting desired gene (e.g., IL-10) into the c-peptide region allows co-secretion of the gene product with insulin. Cas9 lacks the functionalities to effect precision genome edits, which involves efficient incorporation of exogenously supplied single-stranded oligo donor (ssODN) at the break site. However, following the Cas9-induced double-strand break, most cells adopt Non-Homologous End Joining (NHEJ) repair pathway resulting in random insertions and deletions, and little to no incorporation of the desired sequence by Homology-Directed Repair (HDR). Further, induction of double-strand break is genotoxic in primary cells leading to apoptosis via the p53 pathway and potential selection of apoptosis-resistant clones. Further, most CRISPR-technologies are being developed and optimized in the context of cell lines (e.g., HEK293T cells) and these technologies do not necessarily translate well to primary cells. It was proposed to develop a chemically-enhanced Cas9 that enables efficient incorporation of the target gene at the desired locus in human beta cells.

Autoimmune triggered pancreatic β-cell apoptosis is the hallmark of type-1 diabetes. The apoptotic events trigger the macrophage primed T cell activation in the pancreatic lymph node. These activated T-cells secrete chemokines and cytokines that foster the apoptosis of β-cells. Immune modulation in either early stages of T1D development or at the stage of the islet implantation prevents the destruction of β-cells. Previous studies highlighted the protection of β-cells by the overexpression of immunomodulatory cytokine genes that exert pleotropic functions. Increased production of Interleukin-10 (IL-10) using viral vectors protected the β-cells but the viral mediated gene transfer elicited rejection of the grafts in host mice. Previous reports suggest that the ex vivo treatment of islets with IL-6 protected the islets from inflammatory cytokines-induced cell death in vitro and increased their survival and graft function upon transplantation in vivo. An improper regulatory T cells versus effector T cells (Treg/Teff) balance contributes to autoimmune diabetes (T1D). Treatment with low-dose IL-2 expands/activates the Tregs and blocks the actions of Teffs provide immunoregulation without global immunosuppression in T1D. On the other hand, increased production of IL-22 contributes to the β-cell regeneration by cytokine-mediated up-regulation of the β-cell regenerating genes (Reg1 and Reg2). Applicant propose to knock-in these key players of immune regulation to protect and/or enhance the β-cell regeneration using insulin secretion machinery of the beta cells that help in secret immunomodulatory proteins from islet implants.

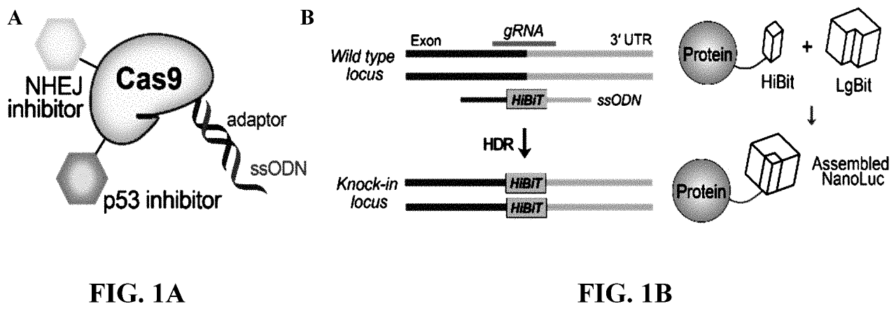

Synthetic chemistry is often deployed to append functionalities or enhance activities of proteins. For example, the conjugation of antibodies to drugs has resulted in a new class of therapeutics, Antibody Drug Conjugates (ADCs), which allow highly localized activation of drugs at the target site. In addition, base-editors display uracil DNA glycosylase inhibitors for local inhibition of these glycosylases, which is a key requirement for efficient base editing. Inspired by the mechanism-of action of antibody-drug conjugates and base editors, Applicants display ssODN, NHEJ and p53 pathway modulators on Cas9 to generate a semi-synthetic, multifunctional genome editor, which is called SynGEM (FIG. 1A). Locating ssODNs close to the DNA break site would enhance the rate of precision genome editing due to the increased local concentration. In addition, inhibiting NHEJ pathways can be a viable strategy to direct DNA repair process toward HDR pathway. Cas9-induced double-strand break leads to activation of p53 pathway followed by apoptosis, greatly reducing the efficiency of precision genome editing in primary cells and stem cells. Selection process to enrich HDR-edited cells may enrich p53-impaired cells, which increase the risk of tumor development when used in clinic. Therefore, temporarily inhibiting p53 pathway by small molecules is another viable strategy for increasing HDR efficiency while lowering genotoxicity. Based on these assumptions, Applicants develop SynGEMs. Long ssODNs are attached to Cas9 by developing a modular conjugation strategy that enables tethering of any ssODN without extra steps. Applicants also append known inhibitors of the NHEJ and p53 pathway to Cas9. Local display of small molecules would minimize the toxicity and mutagenesis due to global NHEJ/p53 pathway inhibition. Towards these goals, Applicants have optimized orthogonal conjugation chemistries to Cas9 and demonstrated HDR enhancement by ssODN tethering to Cas9. Applicants have also validated IL-10 secretion from INS1E cells using Cas9-mediated genome editing.

Development of SynGEM. Applicants first developed a platform for site-specific cysteine conjugation on Cas9. Guided by the structure of Cas9, Applicants engineered cysteines on solvent-exposed loops of various Cas9 domains, and mutated polar residues to minimize potential structure disruption. Eleven single cysteine mutants were recombinantly expressed and using PEG (5 kDa)-maleimide conjugation, it was confirmed that conjugation at 10 sites (M1C, 5204C, E532C, K558C, Q826C, E945C, E1026C, E1068C, 51116C, E1207C) was efficient. Next, Applicants labeled the single-cysteine mutants with short oligonucleotide named ‘universal adaptor’ that can be an anchoring point for any kind of functional molecules based on DNA hybridization (FIG. 1A). Applicants designed a ssODN that would insert a 33-nt DNA fragment (HiBiT sequence) at the GAPDH locus by HDR (FIG. 1B). This insertion resulted in the expression of a fusion protein containing a C-terminal HiBiT tag, a small fragment of the NanoLuc luciferase. Upon cell lysis and complementation with the remainder of NanoLuc, LgBiT, intact NanoLuc was reconstituted eliciting a robust luminescent signal that is proportional to the degree of ssODN insertion (FIG. 1B). Applicants designed two ssODNs that had the same homology arms and insertion sequence, one with a sequence complementary to the adaptor for conjugation and one without for a negative control. Using the negative control ssODN without the complementary sequence, Applicants determined whether appending the DNA adaptor to Cas9 affected the enzyme activity in the HiBiT sequence knock-in assay (not shown). Applicants were able to identify five mutants whose activity was largely maintained (>85% of wildtype in U2OS cells) even after labeling with the 17-nt adaptor.

Next, Applicants proceeded to use Cas9 labeled at those sites and using the luminescence signals from unconjugated ssODN as normalization controls. As described in PCT/US2018/057182, Applicants demonstrated an enhancement in knock-in efficiency by Cas9-ssODN conjugation in multiple cell lines. Finally, appendage of two ssODN significantly improved the degree of HDR. Applicants now confirmed these enhancements in HDR using multiple readouts (e.g., ddPCR) and at several genomic loci without altering the specificity of Cas9. Applicants also engineered Cas9 to accommodate a single sortase recognition sequence (Leu-Pro-Xxx-Thr-Gly, where Xxx is any amino acid)(SEQ ID NO: 9). Expression of these sortase loop-containing Cas9 variants (Cas9-SortLoop) in mammalian cells verified that most retained activity compared to wtCas9, as validated by next-generation sequencing assays quantifying insertion/deletion (indel) mutations events against EVIX1. Applicants were able to confirm sortase-mediated labeling of a model biotin-containing poly-Gly peptide for SortLoop #7. These studies confirm that sortase chemistry can be used for labeling of Cas9 without perturbing activity. Following the demonstration of HDR enhancement using conjugated ssODN to Cas9, Applicants optimized the structure of small-molecule modulators of NHEJ pathway for their activity in cells, synthesized or acquired several known NHEJ inhibitors, and performed preliminary structure-activity relationship studies to determine potential sites for linker attachment. For example, for SCR7 Applicants envisioned that the aryl rings as potential linker attachment site. Applicants synthesized and tested analogs with various aryl rings using a droplet digital (ddPCR) assay which can detect wildtype and genome-edited alleles in RBM20 locus. Applicants saw dose-dependent inhibition of NHEJ as previously reported and DNA-PK inhibitors (KU-57788 and KU-0060648) enhanced HDR enhancement in the HiBIT assay.

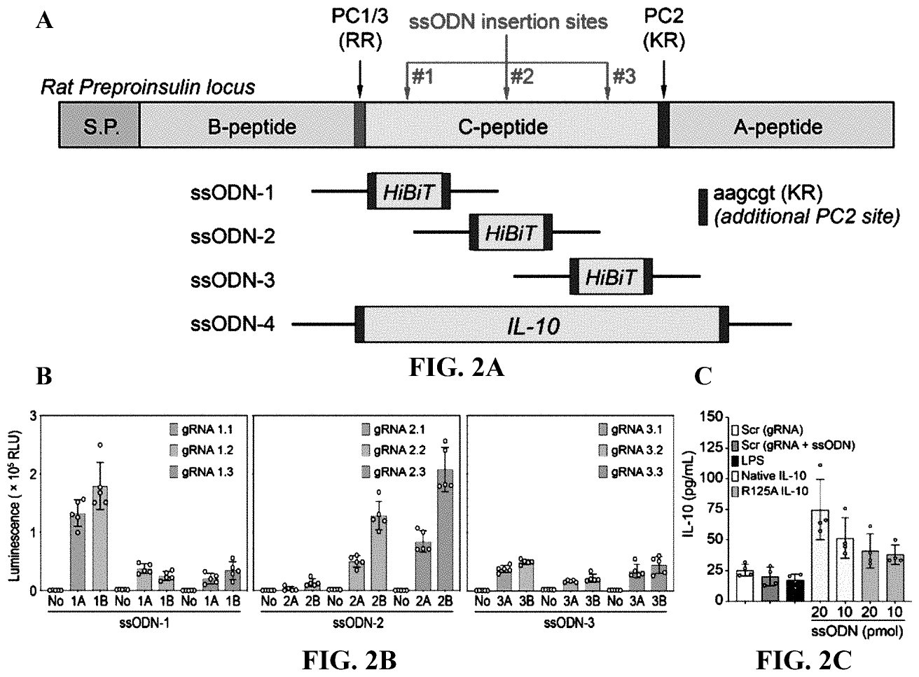

Engineering INS1E cells to secrete IL-10. Applicants demonstrated the HDR-mediated insertion of a gene fragment into c-peptide locus and the resulting secretion of the inserted gene product in INS1E cells. In order to identify the best DNA cleavage site and gene insertion site, Applicants chose three gene insertion sites at the start, middle, and end regions of the c-peptide locus and designed three gRNAs to target these sites (FIG. 2A). Applicants also added an extra protease cleavage sites (Lys-Arg) at the left end or both end of the ssODN for obtaining intact gene product without any extra peptide tags. To identify the optimum gRNA, Applicants used the HiBIT assay described above and examined the luciferase signal in the media of INS1E cells. Applicants found that the secretion of HiBiT product was enhanced when the protease cleavage site was displayed at the both ends of HiBiT (FIG. 2B). Furthermore, Applicants observed higher knock-in efficiency when the cut site and the insertion site are same or as close as possible. The best knock-in conditions corresponded to when the gRNA targets the c-peptide in the middle region and the donor DNA with protease cleavage sites at both the ends are used (FIG. 2B). Based on this optimized design, Applicants set to knock-in IL-10 in INS1E cells. Applicants nucleofected the INS-1E cells with the optimized gRNA and the donor IL-10 ssODNs, and supernatant media was used for the estimation of secreted IL-10 using ELISA. It was observed the efficient insertion and secretion of IL-10 from INS1E cells (FIG. 2C).

Development and optimization of SynGEM in the context of beta cells. Applicants have already identified potent small-molecule inhibitors of the NHEJ pathway proteins, and there exist several inhibitors of p53 pathway that act by inhibiting ATM kinases. Applicants append these inhibitors to Cas9. Based on medicinal chemistry studies, Applicants have identified sites on these small molecules for linker attachment, as previously detailed in PCT/US2018/057182. For NHEJ inhibitors, Applicants synthesized NHEJ inhibitors tested above (e.g., SCR7 analogs, KU-0060648) to bear linkers (e.g., PEG) that are conjugated to Cas9. Applicants generate ˜7 conjugates for each Cas9-ssODN, Cas9-NHEJ inhibitor, and Cas9-p53 pathway inhibitor. Applicants test these conjugates in the ddPCR assay described above to identify the top two conjugates for each category that significantly enhanced HDR and prevent genotoxicity (for p53 inhibitor). ssODN attachment is through adaptors as described in herein. Following the identification of the most optimized systems for ssODN, NHEJ/p53 pathway inhibition, Applicants generate a synthetic Cas9 bearing all the three components. Applicants take multiple orthogonal conjugation strategies: cysteine-maleimide, sortase chemistry, and unnatural amino acids bearing groups with orthogonal reactivity to cysteine and sortase. For unnatural amino acid mutagenesis, Applicants utilized genetic code expansion by adding an engineered pyrrolysyl tRNA (PylT)/tRNA synthetase pair to the translational machinery of cells to enable the site-specific incorporation of p-azido Phenylalanine (pAzF) into CRISPR/Cas9 as described in Example 10 of PCT/US2018/057182. This method relies on a unique codon-tRNA pair and corresponding aminoacyl tRNA synthetase (aaRS) for each unnatural amino acid that does not cross-react with any of the endogenous tRNAs, aaRSs, amino acids or codons in the host organism. The ribosome translates mRNA into a polypeptide by complementing triplet codons with matching aminoacylated tRNAs. Three of the 64 different triplet codons do not code for an amino acid, but cause recruitment of a release factor resulting in disengagement of the ribosome and termination of the synthesis of the growing polypeptide. These codons are called; ochre (TAA), opal (TGA), and amber (TAG). Of the three stop codons, the amber codon is the least used in E. coli (˜7%) and rarely terminates essential genes. Applicants place amber suppression codons at the optimal sites identified above. While Applicants use pAzF as the unnatural amino acid that can react with cyclooctyne group, Applicants also explore tetrazine chemistries which are also high yielding and orthogonal to the reactivity of cysteines, and sortase. Applicants note that multiple reports for incorporation of unnatural amino acids in Cas9 exists and members of the PIs laboratory^{62,63} have deep expertise in unnatural amino acid mutagenesis.

Efficient knock-in of immunomodulatory genes in human islets and human stem cell derived β-cells. Applicants synthesize and optimize SynGEMs in the context of human islets and human stem cell derived beta cells (hSC β-cells). Human islets from cadaver pancreases are obtained from JDRF human islet consortium and will be used as such for the knock-in experiments. Applicants note that multiple methods have now been described for efficient delivery of Cas9:gRNA:ssODN in primary cells, including nucleofection. hSC β-cells are obtained from Prof. Douglas Melton lab at the stage-6 differentiation stage. hSC-β-cells are dissociated first followed by the RNP nucleofection to knock-in the immunomodulatory genes. Applicants perform ELISA to confirm the secretion of the immumodulatory genes. Applicants also employ the NGS based BLESS and GUIDEseq methods to find the off-target effects in the cells. Moreover, functional consequences of IL-10 knock-in are investigated. Applicants investigate the in vitro cytokine release profile from cells loaded in the ceMED and manipulate cell density, device design and reiterate on details of gene insertion such as the insertion site, gRNA and ssODN design to recapitulate the secretion profile required for the desired anti-inflammatory and anti-fibrosis effect in vivo.

### Example 2—Genome Editing of Pancreatic α-Cells to Secrete Functional Molecules

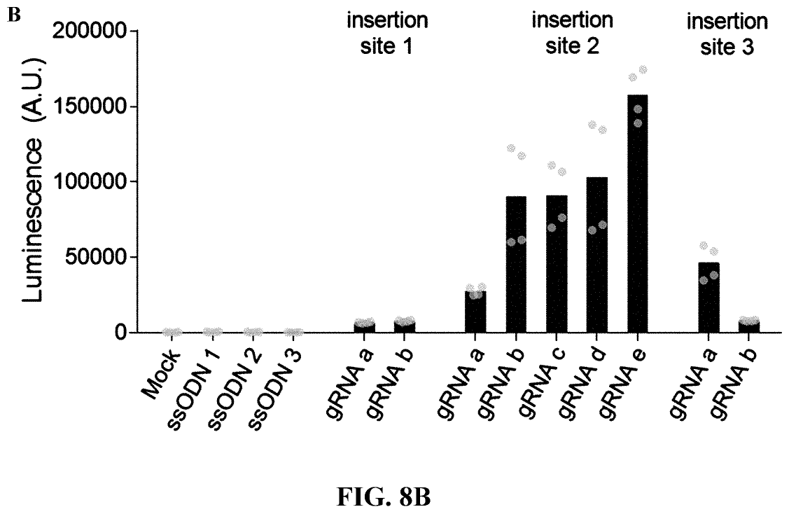

Applicants employed the chemically modified Cas9 to establish a general platform for β-cell genome editing given the urgent need for providing β-cells with immunomodulatory functions, such that cure for the diabetes can be achieved by cell-based therapies. Since c-peptide is cleaved off during insulin processing and secreted, it was hypothesized that knocking in a desired gene into the c-peptide portion of the proinsulin locus would enable the secretion of the inserted gene product. Indeed, a lentiviral vector encoding proinsulin-luciferase fusion construct, which has a Gaussia luciferase in the middle of the c-peptide portion, expressed functional luciferase when stably integrated into the INS-1E β-cell line. Here, the expression level of luciferase was directly proportional to that of insulin, and responded sensitively to external stimuli such as glucose concentration. However, β-cells engineered with viral vectors poses safety issues such as immunogenicity. Thus, precise genome editing can be a powerful way to insert a desired gene fragment into the c-peptide region, which will allow co-secretion of the target gene product with insulin (FIG. 8a).

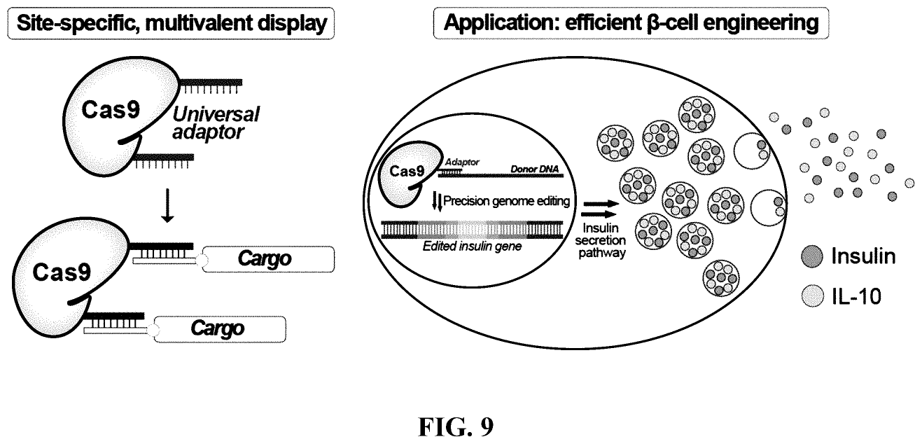

As a proof-of-concept, Applicants first demonstrated the HDR-mediated knock-in of the HiBiT sequence at the c-peptide portion of INS1 locus in INS-1E cells. Target HiBiT sequence was flanked by additional prohormone convertase 2 (PC2) cleavage sites. Therefore, no extra amino acids would be present at each end of the knock-in product after processing (FIG. 8a). To identify the best gene insertion site and DNA cleavage site, three gene insertion sites were chosen at the start, middle, and end regions of the c-peptide locus, and designed several gRNAs to target these sites such that insertion sites and DNA cleavage sites are close enough to obtain high HDR efficiency (FIG. 8a). In addition, genome-wide off-target profiles of gRNAs were considered so that potential off-target sites have mismatches at the seed sequences or have at least three mismatches in the gene-encoding regions. When genome editing was performed at these target sites using Cas9 ribonucleoprotein (RNP) and ssODNs, HiBiT peptide was secreted from INS-1E cells, which could be readily detected through luminescence signals from the cell culture supernatant after complementation by the LgBiT protein. The highest knock-in efficiency was achieved when the c-peptide middle region (site 2) was targeted (FIG. 8b). Therefore, this insertion site was used for the following experiments. HiBiT peptide secretion was stimulated by glucose, indicating that the knock-in product is secreted thought the insulin processing and secretion pathways (FIGS. 8c and 11).

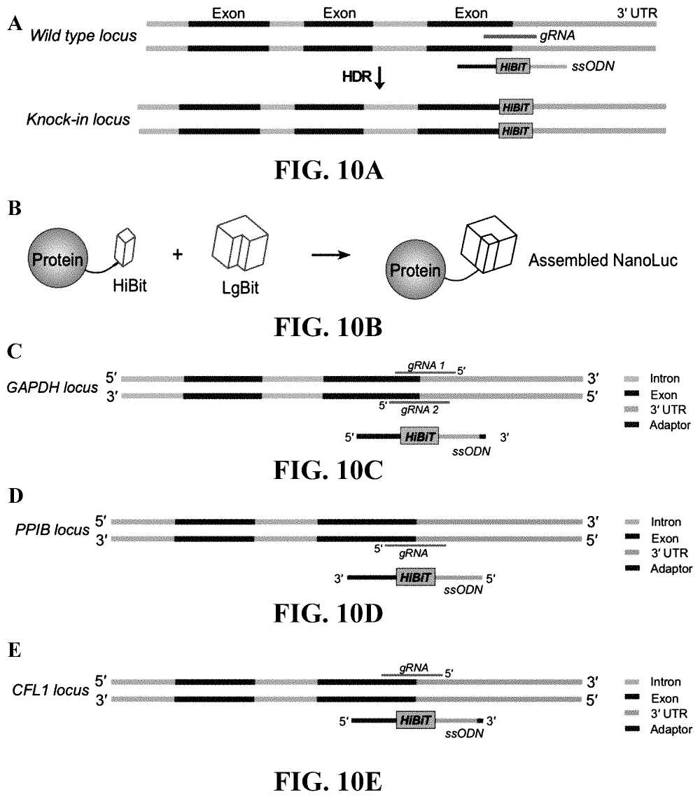

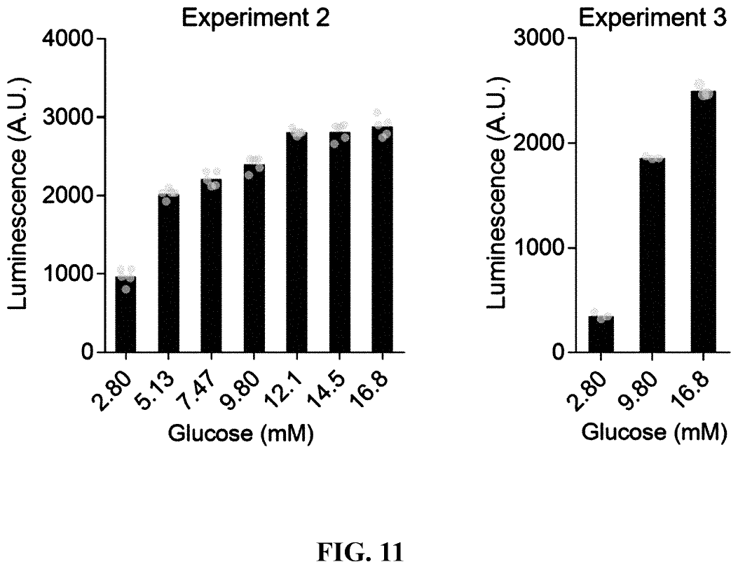

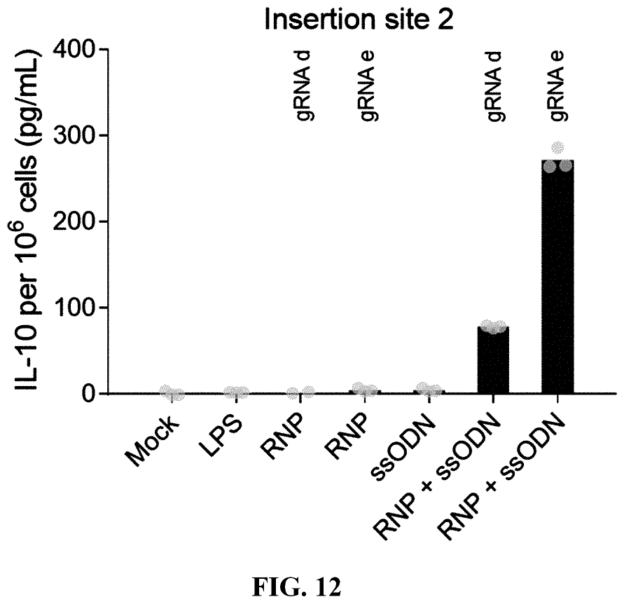

Based on this optimized design, Applicant set out to knock-in a long gene fragment to secret an anti-inflammatory cytokine IL-10 that can protect β-cell from the immune-triggered destruction. The ssODNs for IL-10 insertion had longer homology arms (150 nt left arm and 155 nt right arm) for efficient incorporation of the long gene fragment. Secretory signal peptide sequence present in IL-10 gene was omitted, and only mature protein portion was used because insulin secretion pathway is responsible for the IL-10 secretion at the engineered β-cells. PC2 cleavage sites were added at each end of IL-10 for obtaining intact IL-10 as the knock-in product, and the corresponding ssODN was synthesized by reverse transcription. When INS-1E cells were transfected with both Cas9 RNP and ssODN, IL-10 was secreted to the cell culture media as determined by enzyme-linked immunosorbent assay (ELISA). RNP only nor ssODN only did not induce IL-10 secretion. Moreover, treatment of the cells with lipopolysaccharides (LPS) was not enough to induce IL-10 secretion (FIGS. 8d and 12). Then, genomic DNA was extracted from unedited and edited cells, and amplified the knock-in sequence using the knock-in-specific primer pairs. The IL-10 secretion level correlated with the amount of the edited genomic DNA (FIG. 13), and Sanger sequencing confirmed the correct insertion of the IL-10 gene at the INS1 c-peptide region. Finally, Applicants employed their chemical Cas9 modification system for this β-cell genome editing, and found that both HiBiT secretion and IL-10 secretion were promoted by Cas9-ssODN conjugates (FIGS. 8e, 8f, and 14).

eGFP Disruption Assay to Confirm the Target Specificity of the Cas9-Adaptor Conjugate

Cas9 (10 pmol) and sgRNA (10 pmol) were mixed and incubated at RT for 5 min. U2OS.eGFP.PEST cells' were transfected with the RNP complex using the SE Cell Line 4D-Nucleofector kit (Lonza) following the pulse program of DN-130. After transfection, cells were suspended in the culture media and transferred to a 96-well plate (200,000 cell/well). Forty-eight hours after transfection, cells were fixed with 4% paraformaldehyde solution and nuclei were stained with HCS NuclearMask Blue Stain (Invitrogen). The resulting fluorescence signals from eGFP and nuclei were measured using an ImageXpress Micro High Content Analysis System (Molecular Devices).

### HiBiT Sequence Knock-In by Nucleofection in INS-1E Cells

INS-1E cells were transfected with Cas9 ribonucleoprotein (RNP) and ssODN using the SF Cell Line 4D-Nucleofector kit (Lonza) following the pulse program of DE-130. For Cas9-ssODN conjugates, 20 pmol of Cas9-adaptor were pre-mixed with 20 pmol of ssODN and incubated at RT for 15-30 min prior to RNP formation to ensure Cas9-ssODN conjugate formation. Then 20 pmol of gRNA was added, and the final mixture was incubated for 5-10 min at RT. In cases where Cas9 did not specifically bind ssODNs, the RNP was formed first because nonspecific Cas9-DNA interactions can hamper the RNP formation. After incubating Cas9 and gRNA at RT for 5-10 min, 20 pmol of ssODN were added to the mixture. Approximately 200,000 cells were transfected with the above mixtures in a well of the nucleofection kit, and cells were seeded in a well of a 24-well plate. Cells were incubated at 37° C. for 48 h, and the supernatant was taken to measure the amount of secreted HiBiT peptide using the Nano-Glo HiBiT Extracellular Detection System (Promega). The resulting luminescence signals were normalized based on the cell viability.

### IL-10 Knock-In by Nucleofection in INS-1E Cells

INS-1E cells were transfected with Cas9 ribonucleoprotein (RNP) and ssODN using the SF Cell Line 4D-Nucleofector kit (Lonza) following the pulse program of DE-130. For Cas9-ssODN conjugates, 20 pmol of Cas9-adaptor were pre-mixed with 12 pmol of ssODN and incubated at RT for 15-30 min prior to RNP formation to ensure Cas9-ssODN conjugate formation. Then 20 pmol of gRNA was added, and the final mixture was incubated for 5-10 min at RT. In cases where Cas9 did not specifically bind ssODNs, the RNP was formed first because nonspecific Cas9-DNA interactions can hamper the RNP formation. After incubating Cas9 and gRNA at RT for 5-10 min, 12 pmol of ssODN were added to the mixture. Approximately 200,000 cells were transfected with the above mixtures in a well of the nucleofection kit, and cells were seeded in a well of a 24-well plate. Cells were incubated at 37° C. for 72 h, and the supernatant was taken to measure the amount of secreted IL-10 using the IL-10 Rat ELISA Kit (Invitrogen, catalog #BMS629). The resulting values were normalized based on the cell viability. LPS was used at the concentration of 10 μg/mL (FIGS. 8d and 12).

### Glucose-Stimulated Peptide Secretion

INS-1E cells knocked in with the HiBiT sequence were grown in a large scale. Then, cells were seeded in a 24-well plate at the density of 150,000 cells per well. The next day, cell were washed with and incubated in Krebs-Ringer bicarbonate buffer (138 mM NaCl, 5.4 mM KCl, 5 mM NaHCO₃, 2.6 mM MgCl₂, 2.6 mM CaCl₂), 10 mM HEPES, pH 7.4, 0.5% BSA) without glucose for 2 h. Cells were subsequently incubated with Krebs-Ringer bicarbonate buffer containing glucose (from 2.8 mM to 16.8 mM) for 1 h. The supernatant was taken to measure the amount of secreted HiBiT peptide using the Nano-Glo HiBiT Extracellular Detection System (Promega).

### PCR to Amplify the IL-10 Knock-In Sequence

Genomic DNAs from the edited INS-1E cells were extracted using a DNeasy Blood & Tissue Kit (Qiagen). Fifty ng of genomic DNA was mixed with 1.25 uM of forward primer, 1.25 uM of reverse primer, and Q5 Hot Start High-Fidelity 2× Master Mix (New England Biolabs) in a final volume of 25 μL. Primer set 1 (forward: CCCGGAGAAGCGTAGCAAA, (SEQ ID NO: 10) reverse: CCCCGGCACGCTTATTTTTC (SEQ ID NO: 11), Ta=68° C., 36 temperature cycles) and primer set 2 (forward: CCCGGAGAAGCGTAGCAAAG (SEQ ID NO: 12), reverse: AAGATCCCCGGCACGCTTATTT (SEQ ID NO: 13), Ta=70° C., 40 temperature cycles) were used.

TABLE 1
Primer sequences for gRNA synthesis.

| Primer | |
|---|---|
| name | Primer sequence |
| Universal | AAAAGCACCGACTCGGTGCCACTTTTTCAAGTTGATAACGGACTAGCCTTATTTT |
| reverse | AACTTGCTATTTCTAGCTCTAAAAC (SEQ ID NO: 14) |
| GAPDH | TAATACGACTCACTATAGGTCCAGGGGTCTTACTCCTGTTTTAGAGCTAGAAAT |
| 1 | (SEQ ID NO: 15) |
| forward | |
| GAPDH | TAATACGACTCACTATAGCCTCCAAGGAGTAAGACCCCGTTTTAGAGCTAGAAAT |
| 2 | (SEQ ID NO: 16) |
| forward | |
| PPIB | TAATACGACTCACTATAGCGCCAAGGAGTAGGGCACAGTTTTAGAGCTAGAAAT |
| forward | (SEQ ID NO: 17) |
| CFL1 | TAATACGACTCACTATAGGGCCAGAAGGGGCTCACAAGTTTTAGAGCTAGAAAT |
| forward | (SEQ ID NO: 18) |
| RBM201 | TAATACGACTCACTATA GGGACCTCGGGGAGAGTGACGTTTTAGAGCTAGAAAT |
| | (SEQ ID NO: 19) |
| forward | |
| RBM20 | TAATACGACTCACTATAGGGGAGAGTGACCGGCTCACGTTTTAGAGCTAGAAAT |
| 2 | (SEQ ID NO: 20) |
| forward | |
| INS1 1a | TAATACGACTCACTATAGCCCAAGTCCCGTCGTGAAGGTTTTAGAGCTAGAAAT |
| forward | (SEQ ID NO: 21) |
| INS1 1b | TAATACGACTCACTATAGCTCCAGTTGTGGCACTTGCGTTTTAGAGCTAGAAAT |
| forward | (SEQ ID NO: 22) |
| INS1 2a | TAATACGACTCACTATAGGGTGGAGGCCCGGAGGCCGTTTTAGAGCTAGAAAT |
| forward | (SEQ ID NO: 23) |
| INS1 2b | TAATACGACTCACTATAGGGTGGAGGCCCGGAGGCCGGTTTTAGAGCTAGAAAT |
| forward | (SEQ ID NO: 24) |
| INS1 2c | TAATACGACTCACTATAGTCTGAAGATCCCCGGCCTCGTTTTAGAGCTAGAAAT |
| forward | (SEQ ID NO: 25) |
| INS1 2d | TAATACGACTCACTATAGTGGGTGGAGGCCCGGAGGCGTTTTAGAGCTAGAAAT |
| forward | (SEQ ID NO: 26) |
| INS1 2e | TAATACGACTCACTATAG |
| forward | CTGAAGATCCCCGGCCTCCGTTTTAGAGCTAGAAAT (SEQ ID NO: 27) |
| INS1 3a | TAATACGACTCACTATAGACAATGCCACGCTTCTGCCGTTTTAGAGCTAGAAAT |
| forward | (SEQ ID NO: 28) |
| INS1 3b | TAATACGACTCACTATAGCTTCAGACCTTGGCACTGGGTTTTAGAGCTAGAAAT |
| forward | (SEQ ID NO: 29) |
| eGFP | TAATACGACTCACTATAGGGCACGGGCAGCTTGCCGGGTTTTAGAGCTAGAAAT |
| on-target | (SEQ ID NO: 30) |
| forward | |
| eGFP | TAATACGACTCACTATAGGGCACGGGCAGCTTGCCGCGTTTTAGAGCTAGAAAT |
| off-target | (SEQ ID NO: 31) |
| 1 | |
| forward | |
| eGFP | TAATACGACTCACTATAGGGCACGGGCAGCTTGCCCGGTTTTAGAGCTAGAAAT |
| off-target | (SEQ ID NO: 32) |
| 2 | |
| forward | |
| eGFP | TAATACGACTCACTATAGGGCACGGGCAGCTTCCCGGGTTTTAGAGCTAGAAAT |
| off-target | (SEQ ID NO: 33) |
| 5 | |
| forward | |

TABLE 2
Sequences of short ssODNs (<200 nt).

| ssODN name | Assay | ssODN sequence |
|---|---|---|
| GAPDH | NanoLuc | TCTTCTAGGTATGACAACGAATTTGGCTACAGCAA |
| adaptor | luciferase | CAGGGTGGTGGACCTCATGGCCCACATGGCCTCC |
| | complementation | AAGGAGGTGAGCGGCTGGCGGCTGTTCAAGAAGA |
| | | TTAGCTAAGACCCCTGGACCACCAGCCCCAGCAA |
| | | GAGCACAAGAGGAAGAGAGAGACCCTCACTGCTG |
| | | GGGAGTCCCTGCGACGATGAGAGTGAAGC (SEQ |
| | | ID NO: 34) |
| GAPDH | NanoLuc | TCTTCTAGGTATGACAACGAATTTGGCTACAGCAA |
| adaptor-free | luciferase | CAGGGTGGTGGACCTCATGGCCCACATGGCCTCC |
| | complementation | AAGGAGGTGAGCGGCTGGCGGCTGTTCAAGAAGA |
| | | TTAGCTAAGACCCCTGGACCACCAGCCCCAGCAA |
| | | GAGCACAAGAGGAAGAGAGAGACCCTCACTGCTG |
| | | GGGAGTCCCTGC (SEQ ID NO: 35) |
| GAPDH | NanoLuc | TCTTCTAGGTATGACAACGAATTTGGCTACAGCAA |
| 15-nt adaptor | luciferase | CAGGGTGGTGGACCTCATGGCCCACATGGCCTCC |
| | complementation | AAGGAGGTGAGCGGCTGGCGGCTGTTCAAGAAGA |
| | | TTAGCTAAGACCCCTGGACCACCAGCCCCAGCAA |
| | | GAGCACAAGAGGAAGAGAGAGACCCTCACTGCTG |
| | | GGGAGTCCCTGCGACGATGAGAGTGAA (SEQ ID |
| | | NO: 36) |
| GAPDH | NanoLuc | TCTTCTAGGTATGACAACGAATTTGGCTACAGCAA |
| 13-nt adaptor | luciferase | CAGGGTGGTGGACCTCATGGCCCACATGGCCTCC |
| | complementation | AAGGAGGTGAGCGGCTGGCGGCTGTTCAAGAAGA |
| | | TTAGCTAAGACCCCTGGACCACCAGCCCCAGCAA |
| | | GAGCACAAGAGGAAGAGAGAGACCCTCACTGCTG |
| | | GGGAGTCCCTGCGACGATGAGAGTG (SEQ ID |
| | | NO: 37) |
| PPIB | NanoLuc | CAGCTCAGAGCCCTGTGGCGGACTACAGGGCCTG |
| adaptor | luciferase | CACAGACGGTCACTCAAAGAAAGATGTCCCTGTG |
| | complementation | CCCTAGCTAATCTTCTTGAACAGCCGCCAGCCGCT |
| | | CACCTCCTTGGCGATGGCAAAGGGCTTCTCCACC |
| | | TCGATCTTGCCGCAGTCTGCGATGATCACATCCTT |
| | | CAGGGGTGACGATGAGAGTGAAGC (SEQ ID |
| | | NO: 38) |
| PPIB | NanoLuc | CAGCTCAGAGCCCTGTGGCGGACTACAGGGCCTG |
| adaptor-free | luciferase | CACAGACGGTCACTCAAAGAAAGATGTCCCTGTG |
| | complementation | CCCTAGCTAATCTTCTTGAACAGCCGCCAGCCGCT |
| | | CACCTCCTTGGCGATGGCAAAGGGCTTCTCCACC |
| | | TCGATCTTGCCGCAGTCTGCGATGATCACATCCTT |
| | | CAGGGGT (SEQ ID NO: 39) |
| CFL1 | NanoLuc | GAGGTCAAGGACCGCTGCACCCTGGCAGAGAAG |
| adaptor | luciferase | CTGGGGGGCAGTGCCGTCATCTCCCTGGAGGGC |
| | complementation | AAGCCTTTGGTGAGCGGCTGGCGGCTGTTCAAGA |
| | | AGATTAGCTGAGCCCCTTCTGGCCCCCTGCCTGG |
| | | AGCATCTGGCAGCCCCACACCTGCCCTTGGGGGT |
| | | TGCAGGCTGCCCCCTGACGATGAGAGTGAAGC |
| | | (SEQ ID NO: 40) |
| CFL1 | NanoLuc | GAGGTCAAGGACCGCTGCACCCTGGCAGAGAAG |
| adaptor-free | luciferase | CTGGGGGGCAGTGCCGTCATCTCCCTGGAGGGC |
| | complementation | AAGCCTTTGGTGAGCGGCTGGCGGCTGTTCAAGA |
| | | AGATTAGCTGAGCCCCTTCTGGCCCCCTGCCTGG |
| | | AGCATCTGGCAGCCCCACACCTGCCCTTGGGGGT |
| | | TGCAGGCTGCCCCCT (SEQ ID NO: 41) |
| INS1 | NanoLuc | GGAGGCTCTGTACCTGGTGTGTGGGGAACGTGGT |
| site 1 | luciferase | TTCTTCTACACACCCAAGTCCCGTCGTGAAGTGGA |
| adaptor-free | complementation | GAAGCGTGTGAGCGGCTGGCGGCTGTTCAAGAAG |
| | | ATAGCAAGCGTGACCCGCAAGTGCCACAACTGG |
| | | AGCTGGGTGGAGGCCCGGAGGCCGGGGATCTTC |
| | | AGACCTTGGCACTGG (SEQ ID NO: 42) |
| INS1 | NanoLuc | ACACCCAAGTCCCGTCGTGAAGTGGAGGACCCGC |
| site 2 | luciferase | AAGTGCCACAACTGGAGCTGGGTGGAGGCCCGG |
| adaptor | complementation | AGAAGCGTGTGAGCGGCTGGCGGCTGTTCAAGAA |
| | | GATTAGCAAGCGTGCCGGGGATCTTCAGACCTTG |
| | | GCACTGGAGGTTGCCCGGCAGAAGCGTGGCATTG |
| | | TGGATCAGTGCTGC (SEQ ID NO: 43) |
| INS1 | NanoLuc | ACACCCAAGTCCCGTCGTGAAGTGGAGGACCCGC |
| site 2 | luciferase | AAGTGCCACAACTGGAGCTGGGTGGAGGCCCGG |
| adaptor-free | complementation | AGAAGCGTGTGAGCGGCTGGCGGCTGTTCAAGAA |
| | | GATTAGCAAGCGTGCCGGGGATCTTCAGACCTTG |
| | | GCACTGGAGGTTGCCCGGCAGAAGCGTGGCATTG |
| | | TGGATCAGTGCTGCGACGATGAGAGTGAAGC |
| | | (SEQ ID NO: 44) |
| INS1 | NanoLuc | GCAAGTGCCACAACTGGAGCTGGGTGGAGGCCC |
| site 3 | luciferase | GGAGGCCGGGGATCTTCAGACCTTGGCACTGGAG |
| adaptor-free | complementation | GTTAAGCGTGTGAGCGGCTGGCGGCTGTTCAAGA |
| | | AGATTAGCAAGCGTGCCCGGCAGAAGCGTGGCAT |
| | | TGTGGATCAGTGCTGCACCAGCATCTGCTCCCTCT |
| | | ACCAACTGGAGAACT (SEQ ID NO: 45) |
| GAPDH | GFP | GACAACGAATTTGGCTACAGCAACAGGGTGGTGG |
| adaptor | complementation | ACCTCATGGCCCACATGGCCTCCAAGGAGGGTGG |
| | | CGGCCGTGACCACATGGTCCTTCATGAGTATGTAA |
| | | ATGCTGCTGGGATTACATAAGACCCCTGGACCAC |
| | | CAGCCCCAGCAAGAGCACAAGAGGAAGAGAGAGA |
| | | CCCTCACTGCTGGACGATGAGAGTGAAGC (SEQ |
| | | ID NO: 46) |
| GAPDH | GFP | GACAACGAATTTGGCTACAGCAACAGGGTGGTGG |
| adaptor-free | complementation | ACCTCATGGCCCACATGGCCTCCAAGGAGGGTGG |
| | | CGGCCGTGACCACATGGTCCTTCATGAGTATGTAA |
| | | ATGCTGCTGGGATTACATAAGACCCCTGGACCAC |
| | | CAGCCCCAGCAAGAGCACAAGAGGAAGAGAGAGA |
| | | CCCTCACTGCTG (SEQ ID NO: 47) |
| RBM20 1 | Droplet digital | GTGGGAAGAGCTGCAGGAGGTGAAGCTGGGAGT |
| adaptor | PCR | GTGGGACCTCGGTGAGAGTGACCGGCTCACCGG |
| | | ACTACTAGACCGCGGCCTTTCTGGGCCATATCTGT |
| | | GAGGGAGCCAAGGAGCAGGGACGATGAGAGTGA |
| | | AGC (SEQ ID NO: 48) |
| RBM20 2 | Droplet digital | ACAGATATGGCCCAGAAAGGCCGCGGTCTAGTAG |
| adaptor | PCR | TCCGGTGAGCCGGTCACTGTCCCCGAGGTCCCAC |
| | | ACACCCAGCGACGATGAGAGTGAAGC (SEQ ID |
| | | NO: 49) |

TABLE 3
Sequences of gBlocks DNAs and primers for generating
ssODNs for IL-10 knock-in.

| DNA name | DNA sequence |
|---|---|
| gBlocks | TAATACGACTCACTATAGCTTCACTCTCATCGTCGGCTTTATTCATTGC |
| INS1-1L-10 | AGAGGGGTGGGCGGGGAGTGGTGGACTCAGTTGCAGTAGTTCTCCA |
| adaptor | GTTGGTAGAGGGAGCAGATGCTGGTGCAGCACTGATCCACAATGCCA |
| | CGCTTCTGCCGGGCAACCTCCAGTGCCAAGGTCTGAAGATCCCCGGC |
| | ACGCTTATTTTTCATTTTGAGTGTCACGTAGGCTTCTATGCAGTTGATG |
| | AAGATGTCAAACTCATTCATGGCCTTGTAGACACCTTTGTCTTGGAGC |
| | TTATTAAAATCATTCTTCACCTGCTCCACTGCCTTGCTTTTATTCTCACA |
| | GGGGAGAAATCGATGACAGCGTCGCAGCTGTATCCAGAGGGTCTTCA |
| | GCTTCTCTCCCAGGGAATTCAAATGCTCCTTGATTTCTGGGCCATGGT |
| | TCTCTGCCTGGGGCATCACTTCTACCAGGTAAAACTTGATCATTTCTG |
| | ACAAGGCTTGGCAACCCAAGTAACCCTTAAAGTCCTGCAGTAAGGAAT |
| | CTGTCAGCAGTATGTTGTCCAGCTGGTCCTTCTTTTGAAAGAAAGTCTT |
| | CACTTGACTGAAGGCAGCCCTCAGCTCTCGGAGCATGTGGGTCTGGC |
| | TGACTGGGAAGTGGGTGCAGTTATTGTCACCCCGGATGGAATGGCCT |
| | TTGCTACGCTTCTCCGGGCCTCCACCCAGCTCCAGTTGTGGCACTTG |
| | CGGGTCCTCCACTTCACGACGGGACTTGGGTGTGTAGAAGAAACCAC |
| | GTTCCCCACACACCAGGTACAGAGCCTCCACCAGGTGAGGACCACAA |
| | AGGTGCTGTTTGACAAAAGC (SEQ ID NO: 50) |
| gBlocks | TAATACGACTCACTATAGGCTTTATTCATTGCAGAGGGGTGGGCGGG |
| INS1-1L-10 | GAGTGGTGGACTCAGTTGCAGTAGTTCTCCAGTTGGTAGAGGGAGCA |
| adaptor-free | GATGCTGGTGCAGCACTGATCCACAATGCCACGCTTCTGCCGGGCAA |
| | CCTCCAGTGCCAAGGTCTGAAGATCCCCGGCACGCTTATTTTTCATTT |
| | TGAGTGTCACGTAGGCTTCTATGCAGTTGATGAAGATGTCAAACTCAT |
| | TCATGGCCTTGTAGACACCTTTGTCTTGGAGCTTATTAAAATCATTCTT |
| | CACCTGCTCCACTGCCTTGCTTTTATTCTCACAGGGGAGAAATCGATG |
| | ACAGCGTCGCAGCTGTATCCAGAGGGTCTTCAGCTTCTCTCCCAGGG |
| | AATTCAAATGCTCCTTGATTTCTGGGCCATGGTTCTCTGCCTGGGGCA |
| | TCACTTCTACCAGGTAAAACTTGATCATTTCTGACAAGGCTTGGCAAC |
| | CCAAGTAACCCTTAAAGTCCTGCAGTAAGGAATCTGTCAGCAGTATGT |
| | TGTCCAGCTGGTCCTTCTTTTGAAAGAAAGTCTTCACTTGACTGAAGG |
| | CAGCCCTCAGCTCTCGGAGCATGTGGGTCTGGCTGACTGGGAAGTGG |
| | GTGCAGTTATTGTCACCCCGGATGGAATGGCCTTTGCTACGCTTCTCC |
| | GGGCCTCCACCCAGCTCCAGTTGTGGCACTTGCGGGTCCTCCACTTC |
| | ACGACGGGACTTGGGTGTGTAGAAGAAACCACGTTCCCCACACACCA |
| | GGTACAGAGCCTCCACCAGGTGAGGACCACAAAGGTGCTGTTTGACA |
| | AAAGC (SEQ ID NO: 51) |
| INS1 forward | TAATACGACTCACTATAGCTTCACTCTCATCG (SEQ ID NO: 52) |
| adaptor | |
| INS1 forward | TAATACGACTCACTATAGGCTTTATTCATTGCAGAGGGGTGG (SEQ ID |
| adaptor-free | NO: 53) |
| INS1 reverse | GCTTTTGTCAAACAGCACCTT (SEQ ID NO: 54) |
| universal | |

TABLE 4
Sequences of long ssODNs for IL-10 knock-in.

| ssODN name | Assay | ssODN sequence |
|---|---|---|
| INS1-1-10 | IL-10 ELISA | GCTTTTGTCAAACAGCACCTTTGTGGTCCTCACCTG |
| adaptor | | GTGGAGGCTCTGTACCTGGTGTGTGGGGAACGTGG |
| | | TTTCTTCTACACACCCAAGTCCCGTCGTGAAGTGGA |
| | | GGACCCGCAAGTGCCACAACTGGAGCTGGGTGGAG |
| | | GCCCGGAGAAGCGTAGCAAAGGCCATTCCATCCGG |
| | | GGTGACAATAACTGCACCCACTTCCCAGTCAGCCAG |
| | | ACCCACATGCTCCGAGAGCTGAGGGCTGCCTTCAG |
| | | TCAAGTGAAGACTTTCTTTCAAAAGAAGGACCAGCT |
| | | GGACAACATACTGCTGACAGATTCCTTACTGCAGGA |
| | | CTTTAAGGGTTACTTGGGTTGCCAAGCCTTGTCAGA |
| | | AATGATCAAGTTTTACCTGGTAGAAGTGATGCCCCA |
| | | GGCAGAGAACCATGGCCCAGAAATCAAGGAGCATTT |
| | | GAATTCCCTGGGAGAGAAGCTGAAGACCCTCTGGAT |
| | | ACAGCTGCGACGCTGTCATCGATTTCTCCCCTGTGA |
| | | GAATAAAAGCAAGGCAGTGGAGCAGGTGAAGAATG |
| | | ATTTTAATAAGCTCCAAGACAAAGGTGTCTACAAGG |
| | | CCATGAATGAGTTTGACATCTTCATCAACTGCATAGA |
| | | AGCCTACGTGACACTCAAAATGAAAAATAAGCGTGC |
| | | CGGGGATCTTCAGACCTTGGCACTGGAGGTTGCCC |
| | | GGCAGAAGCGTGGCATTGTGGATCAGTGCTGCACC |
| | | AGCATCTGCTCCCTCTACCAACTGGAGAACTACTGC |
| | | AACTGAGTCCACCACTCCCCGCCCACCCCTCTGCAA |
| | | TGAATAAAGCCGACGATGAGAGTGAAGC (SEQ ID |
| | | NO: 55) |
| INS1-1-10 | IL-10 ELISA | GCTTTTGTCAAACAGCACCTTTGTGGTCCTCACCTG |
| adaptor-free | | GTGGAGGCTCTGTACCTGGTGTGTGGGGAACGTGG |
| | | TTTCTTCTACACACCCAAGTCCCGTCGTGAAGTGGA |
| | | GGACCCGCAAGTGCCACAACTGGAGCTGGGTGGAG |
| | | GCCCGGAGAAGCGTAGCAAAGGCCATTCCATCCGG |
| | | GGTGACAATAACTGCACCCACTTCCCAGTCAGCCAG |
| | | ACCCACATGCTCCGAGAGCTGAGGGCTGCCTTCAG |
| | | TCAAGTGAAGACTTTCTTTCAAAAGAAGGACCAGCT |
| | | GGACAACATACTGCTGACAGATTCCTTACTGCAGGA |
| | | CTTTAAGGGTTACTTGGGTTGCCAAGCCTTGTCAGA |
| | | AATGATCAAGTTTTACCTGGTAGAAGTGATGCCCCA |
| | | GGCAGAGAACCATGGCCCAGAAATCAAGGAGCATTT |
| | | GAATTCCCTGGGAGAGAAGCTGAAGACCCTCTGGAT |
| | | ACAGCTGCGACGCTGTCATCGATTTCTCCCCTGTGA |
| | | GAATAAAAGCAAGGCAGTGGAGCAGGTGAAGAATG |
| | | ATTTTAATAAGCTCCAAGACAAAGGTGTCTACAAGG |
| | | CCATGAATGAGTTTGACATCTTCATCAACTGCATAGA |
| | | AGCCTACGTGACACTCAAAATGAAAAATAAGCGTGC |
| | | CGGGGATCTTCAGACCTTGGCACTGGAGGTTGCCC |
| | | GGCAGAAGCGTGGCATTGTGGATCAGTGCTGCACC |
| | | AGCATCTGCTCCCTCTACCAACTGGAGAACTACTGC |
| | | AACTGAGTCCACCACTCCCCGCCCACCCCTCTGCAA |
| | | TGAATAAAGCC (SEQ ID NO: 56) |

The following references pertain to Examples 1-2.
1. Wang, H.; La Russa, M.; Qi, L. S., CRISPR/Cas9 in Genome Editing and Beyond. Annu. Rev. Biochem. 2016, 85, 227-264.
2. Wang, H. X.; Li, M.; Lee, C. M.; Chakraborty, S.; Kim, H. W.; Bao, G.; Leong, K. W., CRISPR/Cas9-Based Genome Editing for Disease Modeling and Therapy: Challenges and Opportunities for Nonviral Delivery. Chem. Rev. 2017, 117, 9874-9906.
3. Knott, G. J.; Doudna, J. A., CRISPR-Cas guides the future of genetic engineering. Science 2018, 361, 866-869.
4. Zhang, K. X.; Deng, R. J.; Teng, X. C.; Li, Y.; Sun, Y. P.; Ren, X. J.; Li, J. H., Direct Visualization of Single-Nucleotide Variation in mtDNA Using a CRISPR/Cas9-Mediated Proximity Ligation Assay. J. Am. Chem. Soc. 2018, 140, 11293-11301.
5. Thakore, P. I.; Black, J. B.; Hilton, I. B.; Gersbach, C. A., Editing the epigenome: technologies for programmable transcription and epigenetic modulation. Nat. Methods 2016, 13, 127-37.
6. Adli, M., The CRISPR tool kit for genome editing and beyond. Nat. Commun. 2018, 9, 1911.
7. Knight, S. C.; Tjian, R.; Doudna, J. A., Genomes in Focus: Development and Applications of CRISPR-Cas9 Imaging Technologies. Angew. Chem. Int. Edit. 2018, 57, 4329-4337.
8. Richardson, C. D.; Ray, G. J.; DeWitt, M. A.; Curie, G. L.; Corn, J. E., Enhancing homology-directed genome editing by catalytically active and inactive CRISPR-Cas9 using asymmetric donor DNA. Nat. Biotechnol. 2016, 34 (3), 339-344.
9. Bollen, Y.; Post, J.; Koo, B. K.; Snippert, H. J. G., How to create state-of-the-art genetic model systems: strategies for optimal CRISPR-mediated genome editing. Nucleic Acids Res. 2018, 46, 6435-6454.
10. Pawelczak, K. S.; Gavande, N. S.; VanderVere-Carozza, P. S.; Turchi, J. J., Modulating DNA Repair Pathways to Improve Precision Genome Engineering. ACS Chem. Biol. 2018, 13, 389-396.
11. Raper, A. T.; Stephenson, A. A.; Suo, Z. C., Sharpening the Scissors: Mechanistic Details of CRISPR/Cas9 Improve Functional Understanding and Inspire Future Research. J. Am. Chem. Soc. 2018, 140, 11142-11152.
12. Yang, Y.; Wang, L.; Bell, P.; McMenamin, D.; He, Z.; White, J.; Yu, H.; Xu, C.; Morizono, H.; Musunuru, K.; Batshaw, M. L.; Wilson, J. M., A dual AAV system enables the Cas9-mediated correction of a metabolic liver disease in newborn mice. Nat. Biotechnol. 2016, 34, 334-338.
13. Kosicki, M.; Tomberg, K.; Bradley, A., Repair of double-strand breaks induced by CRISPR-Cas9 leads to large deletions and complex rearrangements. Nat. Biotechnol. 2018, 36, 765-771.
14. Gu, B.; Posfai, E.; Rossant, J., Efficient generation of targeted large insertions by microinjection into two-cell-stage mouse embryos. Nat. Biotechnol. 2018, 36, 632-637.
15. Charlesworth, C. T.; Deshpande, P. S.; Dever, D. P.; Dejene, B.; Gomez-Ospina, N.; Mantri, S.; Pavel-Dinu, M.; Camarena, J.; Weinberg, K. I.; Porteus, M. H., Identification of Pre-Existing Adaptive Immunity to Cas9 Proteins in Humans. bioRxiv 2018, 243345.
16. Boboila, C.; Jankovic, M.; Yan, C. T.; Wang, J. H.; Wesemann, D. R.; Zhang, T. T.; Fazeli, A.; Feldman, L.; Nussenzweig, A.; Nussenzweig, M.; Alt, F. W., Alternative end-joining catalyzes robust IgH locus deletions and translocations in the combined absence of ligase 4 and Ku70. Proc. Natl. Acad. Sci. USA 2010, 107, 3034-3039.
17. Sallmyr, A.; Tomkinson, A. E., Repair of DNA double-strand breaks by mammalian alternative end-joining pathways. J. Biol. Chem. 2018, 293, 10536-10546.
18. Komor, A. C.; Badran, A. H.; Liu, D. R., Editing the Genome Without Double-Stranded DNA Breaks. ACS Chem. Biol. 2018, 13, 383-388.
19. Komor, A. C.; Badran, A. H.; Liu, D. R., Editing the Genome Without Double-Stranded DNA Breaks. ACS chemical biology 2018, 13 (2), 383-388.
20. Haapaniemi, E.; Botla, S.; Persson, J.; Schmierer, B.; Taipale, J., CRISPR-Cas9 genome editing induces a p53-mediated DNA damage response. Nat. Med. 2018, 24, 927-930.
21. Ihry, R. J.; Worringer, K. A.; Salick, M. R.; Frias, E.; Ho, D.; Theriault, K.; Kommineni, S.; Chen, J.; Sondey, M.; Ye, C.; Randhawa, R.; Kulkarni, T.; Yang, Z.; McAllister, G.; Russ, C.; Reece-Hoyes, J.; Forrester, W.; Hoffman, G. R.; Dolmetsch, R.; Kaykas, A., p53 inhibits CRISPR-Cas9 engineering in human pluripotent stem cells. Nat. Med. 2018, 24, 939-946.
22. Rouet, R.; Thuma, B. A.; Roy, M. D.; Lintner, N. G.; Rubitski, D. M.; Finley, J. E.; Wisniewska, H. M.; Mendonsa, R.; Hirsh, A.; de Onate, L.; Compte Barron, J.; McLellan, T. J.; Bellenger, J.; Feng, X.; Varghese, A.; Chrunyk, B. A.; Borzilleri, K.; Hesp, K. D.; Zhou, K.; Ma, N.; Tu, M.; Dullea, R.; McClure, K. F.; Wilson, R. C.; Liras, S.; Mascitti, V.; Doudna, J. A., Receptor-Mediated Delivery of CRISPR-Cas9 Endonuclease for Cell-Type-Specific Gene Editing. J. Am. Chem. Soc. 2018, 140, 6596-6603.
23. Koniev, 0.; Wagner, A., Developments and recent advancements in the field of endogenous amino acid selective bond forming reactions for bioconjugation. Chem. Soc. Rev. 2015, 44, 5495-5551.
24. Engelen, W.; Janssen, B. M.; Merkx, M., DNA-based control of protein activity. Chem. Commun. 2016, 52, 3598-3610.
25. Gamble, A.; Pepper, A. R.; Bruni, A.; Shapiro, A. M. J., The journey of islet cell transplantation and future development. Islets 2018, 10 (2), 80-94.
26. Sneddon, J. B.; Tang, Q.; Stock, P.; Bluestone, J. A.; Roy, S.; Desai, T.; Hebrok, M., Stem Cell Therapies for Treating Diabetes: Progress and Remaining Challenges. Cell Stem Cell 2018, 22 (6), 810-823.
27. Song, S.; Roy, S., Progress and challenges in macroencapsulation approaches for type 1 diabetes (T1D) treatment: Cells, biomaterials, and devices. Biotechnol Bioeng 2016, 113 (7), 1381-1402.
28. An, D.; Chiu, A.; Flanders, J. A.; Song, W.; Shou, D. H.; Lu, Y. C.; Grunnet, L. G.; Winkel, L.; Ingvorsen, C.; Christophersen, N. S.; Fels, J. J.; Sand, F. W.; Ji, Y. W.; Qi, L.; Pardo, Y.; Luo, D.; Silberstein, M.; Fan, J. T.; Ma, M. L., Designing a retrievable and scalable cell encapsulation device for potential treatment of type 1 diabetes. Proc. Natl. Acad. Sci. USA 2018, 115 (2), E263-E272.
29. Souza, K. L. A.; Gurgul-Convey, E.; Elsner, M.; Lenzen, S., Interaction between pro-inflammatory and anti-inflammatory cytokines in insulin-producing cells. J Endocrinol 2008, 197 (1), 139-150.
30. Carter, J. D.; Ellett, J. D.; Chen, M.; Smith, K. M.; Fialkow, L. B.; McDuffie, M. J.; Tung, K. S.; Nadler, J. L.; Yang, Z. D., Viral IL-10-mediated immune regulation in pancreatic islet transplantation. Mol Ther 2005, 12 (2), 360-368.
31. Russell, M. A.; Morgan, N. G., The impact of anti-inflammatory cytokines on the pancreatic beta-cell. Islets 2014, 6 (3), e950547.
32. Huurman, V. A. L.; Velthuis, J. H. L.; Hilbrands, R.; Tree, T. I. M.; Gillard, P.; van der Meer-Prins, P. M. W.; Duinkerken, G.; Pinkse, G. G. M.; Keymeulen, B.; Roelen, D. L.; Claas, F. H. J.; Pipeleers, D. G.; Roep, B. O., Allograft-Specific Cytokine Profiles Associate with Clinical Outcome After Islet Cell Transplantation. Am J Transplant 2009, 9 (2), 382-388.
33. Wang, K.; Yu, L. Y.; Jiang, L. Y.; Wang, H. B.; Wang, C. Y.; Luo, Y., The paracrine effects of adipose-derived stem cells on neovascularization and biocompatibility of a macroencapsulation device. Acta Biomater 2015, 15, 65-76.
34. Jinek, M.; Jiang, F.; Taylor, D. W.; Sternberg, S. H.; Kaya, E.; Ma, E.; Anders, C.; Hauer, M.; Zhou, K.; Lin, S.; Kaplan, M.; Iavarone, A. T.; Charpentier, E.; Nogales, E.; Doudna, J. A., Structures of Cas9 endonucleases reveal RNA-mediated conformational activation. Science 2014, 343, 1247997.
35. Jiang, F. G.; Zhou, K. H.; Ma, L. L.; Gressel, S.; Doudna, J. A., A Cas9-guide RNA complex preorganized for target DNA recognition. Science 2015, 348, 1477-1481.
36. Jiang, F.; Taylor, D. W.; Chen, J. S.; Kornfeld, J. E.; Zhou, K.; Thompson, A. J.; Nogales, E.; Doudna, J. A., Structures of a CRISPR-Cas9 R-loop complex primed for DNA cleavage. Science 2016, 351, 867-871.
37. Schwinn, M. K.; Machleidt, T.; Zimmerman, K.; Eggers, C. T.; Dixon, A. S.; Hurst, R.; Hall, M. P.; Encell, L. P.; Binkowski, B. F.; Wood, K. V., CRISPR-Mediated Tagging of Endogenous Proteins with a Luminescent Peptide. ACS Chem. Biol. 2018, 13, 467-474.
38. Fu, Y.; Foden, J. A.; Khayter, C.; Maeder, M. L.; Reyon, D.; Joung, J. K.; Sander, J. D., High-frequency off-target mutagenesis induced by CRISPR-Cas nucleases in human cells. Nat. Biotechnol. 2013, 31, 822-826.
39. Kamiyama, D.; Sekine, S.; Barsi-Rhyne, B.; Hu, J.; Chen, B.; Gilbert, L. A.; Ishikawa, H.; Leonetti, M. D.; Marshall, W. F.; Weissman, J. S.; Huang, B., Versatile protein tagging in cells with split fluorescent protein. Nat. Commun. 2016, 7, 11046.
40. Leonetti, M. D.; Sekine, S.; Kamiyama, D.; Weissman, J. S.; Huang, B., A scalable strategy for high-throughput GFP tagging of endogenous human proteins. Proc. Natl. Acad. Sci. USA 2016, 113, E3501-E3508.
41. Miyaoka, Y.; Berman, J. R.; Cooper, S. B.; Mayerl, S. J.; Chan, A. H.; Zhang, B.; Karlin-Neumann, G. A.; Conklin, B. R., Systematic quantification of HDR and NHEJ reveals effects of locus, nuclease, and cell type on genome-editing. Sci. Rep. 2016, 6, 23549.
42. Kato-Inui, T.; Takahashi, G.; Hsu, S.; Miyaoka, Y., Clustered regularly interspaced short palindromic repeats (CRISPR)/CRISPR-associated protein 9 with improved proof-reading enhances homology-directed repair. Nucleic Acids Res. 2018, 46, 4677-4688.
43. Eric J. Aird, K. N. L., Amber St. Martin, Reuben S. Harris, Wendy R. Gordon Increasing Cas9-mediated homology-directed repair efficiency through covalent tethering of DNA repair template. Commun. Biol. 2018, 1, 54.
44. Savic, N.; Ringnalda, F. C. A. S.; Lindsay, H.; Berk, C.; Bargsten, K.; Li, Y. Z.; Neri, D.; Robinson, M. D.; Ciaudo, C.; Hall, J.; Jinek, M.; Schwank, G., Covalent linkage of the DNA repair template to the CRISPR-Cas9 nuclease enhances homology-directed repair. Elife 2018, 7, e33761.
45. Ma, M.; Zhuang, F.; Hu, X.; Wang, B.; Wen, X. Z.; Ji, J. F.; Xi, J. J., Efficient generation of mice carrying homozygous double-floxp alleles using the Cas9-Avidin/Biotin-donor DNA system. Cell Res 2017, 27 (4), 578-581.
46. Gu, B.; Posfai, E.; Rossant, J., Efficient generation of targeted large insertions by microinjection into two-cell-stage mouse embryos. Nat. Biotechnol. 2018, 36 (7), 632-+.
47. Burns, S. M.; Vetere, A.; Walpita, D.; Dancik, V.; Khodier, C.; Perez, J.; Clemons, P. A.; Wagner, B. K; Altshuler, D., High-throughput luminescent reporter of insulin secretion for discovering regulators of pancreatic Beta-cell function. Cell Metab 2015, 21 (1), 126-37.
48. Johannesson, B.; Sui, L.; Freytes, D. O.; Creusot, R. J.; Egli, D., Toward beta cell replacement for diabetes. EMBO J 2015, 34 (7), 841-55.
49. Bettini, M.; Castellaw, A. H.; Lennon, G. P.; Burton, A. R.; Vignali, D. A., Prevention of autoimmune diabetes by ectopic pancreatic beta-cell expression of interleukin-35. Diabetes 2012, 61 (6), 1519-26.
50. Brauch, K. M.; Karst, M. L.; Herron, K. J.; de Andrade, M.; Pellikka, P. A.; Rodeheffer, R. J.; Michels, V. V.; Olson, T. M., Mutations in ribonucleic acid binding protein gene cause familial dilated cardiomyopathy. J. Am. Coll. Cardiol. 2009, 54, 930-941.
51. Brauch, K. M.; Karst, M. L.; Herron, K. J.; de Andrade, M.; Pellikka, P. A.; Rodeheffer, R. J.; Michels, V. V.; Olson, T. M., Mutations in ribonucleic acid binding protein gene cause familial dilated cardiomyopathy. J Am Coll Cardiol 2009, 54 (10), 930-41.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered cell comprising one or more polynucleotides encoding:
a. one or more secreted proteins, or a functional fragment thereof, wherein the one or more secreted proteins or functional fragments thereof are inserted in one or more endogenous genes of the cell, the one or more endogenous genes encoding a secretory protein or peptide; and
b. an optional co-expressed protein that enhances the expression, stability or biological activity of the secreted protein.

2. The cell of claim 1, wherein the secreted protein or peptide is an immunomodulatory protein, an anti-fibrotic, a hormone, anti-microbial or a protein or peptide that promotes tissue regeneration.

3. The cell of claim 2, wherein the secreted protein is an immunomodulatory protein and the co-expressed protein is an anti-fibrotic protein.

4. The cell of claim 2, wherein the immunomodulatory protein is a cytokine.

5. The cell of claim 2, wherein the immunomodulatory protein is IL-2, IL-4, IL-6, IL-10, IL-15, or IL-22.

6. The cell of claim 2, wherein the secreted protein is an anti-fibrotic protein, optionally wherein the anti-fibrotic protein is a peroxisome proliferator-activated receptor.

7. The cell of claim 2, wherein the secreted protein is a protein or peptide that promotes tissue regeneration, optionally Reg1A, Reg1B, Reg2, Reg3A, Reg3g, and/or Reg 4, or a combination thereof.

8. The cell of claim 2, wherein the secreted protein is a hormone.

9. The cell of claim 8, wherein the hormone is insulin.

10. The cell of claim 8, wherein the hormone is a neuroendocrine hormone, optionally selected from thyrotropin-releasing hormone, corticotropin-releasing hormone, histamine, growth hormone-releasing hormone, somatostatin, gonadotropin-releasing hormone, serotonin, dopamine, neurotensin, oxytocin, vasopressin, epinephrine, and norepinephrine.

11. The cell of claim 2, wherein the secreted protein is an anti-microbial, optionally wherein the anti-microbial is α-defensin HD-6, HNP-1 and β-defensin hBD-3

12. The cell of claim 1, wherein the cell is a eukaryotic cell.

13. The cell of claim 1, wherein the cell is a human cell.

14. The cell of claim 1, wherein the cell is an α a cell, L cell, stem cell, or β cell.

15. The cell of claim 1, wherein the cell is a pancreatic cell.

16. The cell of claim 15, wherein the pancreatic cell is a β cell differentiated from a progenitor in vitro.

17. The cell of claim 1, wherein the cell is a stem cell.

18. The cell of claim 1, wherein the cell is a primary cell.

19. The cell of claim 1, wherein the cell is a plant cell.

20. The cell of claim 1, further comprising one or more polynucleotides encoding a protein promoting pancreatic β cell regeneration.

21. The cell of claim 20, wherein the protein is a cytokine.

22. The cell of claim 21, wherein the cytokine is IL-22.

23. The cell of claim 20, wherein the protein upregulates one or more genes promoting pancreatic β cell regeneration.

24. The cell of claim 23, wherein the one or more genes promoting pancreatic β cell regeneration is Reg1, Reg2, or a combination thereof.

25. The cell of claim 1, further comprising one or more polynucleotides encoding interference RNA.

26. The cell of claim 25, wherein the interference RNA is siRNA.

27. The cell of claim 25, wherein the interference RNA suppresses TGF-β, colony-stimulating factor 1, or a combination thereof.

28. The cell of claim 1, wherein the one or more inserted polynucleotides comprise one or more protease cleavage sites.

29. The cell of claim 28, wherein the one or more inserted polynucleotides comprise a protease cleavage site on each end.

30. The cell of claim 1, wherein the secretory protein or peptide is c-peptide.

31. A method of treating a disease comprising administering a cell of claim 1 to a subject in need thereof.

32. The method of claim 31, wherein the disease is diabetes.

33. A method of making the cell of claim 1, comprising
a. obtaining the cell from an organism;
b. editing the cell to insert one or more polynucleotides, wherein insertion is in-frame with the secretory protein.

34. The method of claim 33, wherein the editing the cell is with a nucleic acid editing system.

35. The method of claim 34, wherein the nucleic acid editing system is selected from a CRISPR nucleic acid editing system, zinc finger nuclease, TALEN, meganuclease, or a Semisynthetic Genome Editing Multifunctional system (Syngem).

36. The method of claim 33, wherein the one or more polynucleotides are inserted in the exon portion of the secretory protein.

37. The method of claim 33, wherein the editing is performed ex-vivo.

38. The method of claim 33, further comprising the step of inserting the cell into an organism.

39. The method of claim 38, wherein the organism is the same organism.

40. The method of claim 38, wherein inserting the cell is by infusion.

41. The method of claim 34, wherein the peptide is a c-peptide.

42. The method of claim 41, wherein the editing the cell comprises using the CRISPR editing system comprising a gRNA that targets the c-peptide in the middle region of the c-peptide.


SEQUENCE LISTING (56 sequences)

SEQ ID NO: 1 - protein, 5 aa, Artificial Sequence
  Synthetic
VITYF

SEQ ID NO: 2 - protein, 5 aa, Artificial Sequence
  Synthetic
VVVVV

SEQ ID NO: 3 - protein, 7 aa, Artificial Sequence
  Synthetic
KPWWPRR

SEQ ID NO: 4 - protein, 9 aa, Artificial Sequence
  Synthetic
NIVNVSLVK

SEQ ID NO: 5 - protein, 5 aa, Artificial Sequence
  Synthetic
LPFFD

SEQ ID NO: 6 - protein, 7 aa, Artificial Sequence
  Synthetic
PGKLVYA

SEQ ID NO: 7 - protein, 7 aa, Artificial Sequence
  Synthetic
LVEALYL

SEQ ID NO: 8 - protein, 6 aa, Artificial Sequence
  Synthetic
RGFFYT

SEQ ID NO: 9 - protein, 5 aa, Artificial Sequence
  Synthetic
  misc_feature (3)..(3): Xaa can be any naturally occurring amino acid
LPXTG

SEQ ID NO: 10 - DNA, 19 bp, Artificial Sequence
  Synthetic
cccggagaagcgtagcaaa

SEQ ID NO: 11 - DNA, 20 bp, Artificial Sequence
  Synthetic
ccccggcacgcttatttttc

SEQ ID NO: 12 - DNA, 20 bp, Artificial Sequence
  Synthetic
cccggagaagcgtagcaaag

SEQ ID NO: 13 - DNA, 22 bp, Artificial Sequence
  Synthetic
aagatccccggcacgcttattt

SEQ ID NO: 14 - DNA, 80 bp, Artificial Sequence
  Synthetic
aaaagcaccgactcggtgccactttttcaagttgataacggactagccttattttaacttgctatttctagctctaaaac

SEQ ID NO: 15 - DNA, 54 bp, Artificial Sequence
  Synthetic
taatacgactcactataggtccaggggtcttactcctgttttagagctagaaat

SEQ ID NO: 16 - DNA, 55 bp, Artificial Sequence
  Synthetic
taatacgactcactatagcctccaaggagtaagaccccgttttagagctagaaat

SEQ ID NO: 17 - DNA, 54 bp, Artificial Sequence
  Synthetic
taatacgactcactatagcgccaaggagtagggcacagttttagagctagaaat

SEQ ID NO: 18 - DNA, 54 bp, Artificial Sequence
  Synthetic
taatacgactcactatagggccagaaggggctcacaagttttagagctagaaat

SEQ ID NO: 19 - DNA, 54 bp, Artificial Sequence
  Synthetic
taatacgactcactatagggacctcggggagagtgacgttttagagctagaaat

SEQ ID NO: 20 - DNA, 54 bp, Artificial Sequence
  Synthetic
taatacgactcactataggggagagtgaccggctcacgttttagagctagaaat

SEQ ID NO: 21 - DNA, 54 bp, Artificial Sequence
  Synthetic
taatacgactcactatagcccaagtcccgtcgtgaaggttttagagctagaaat

SEQ ID NO: 22 - DNA, 54 bp, Artificial Sequence
  Synthetic
taatacgactcactatagctccagttgtggcacttgcgttttagagctagaaat

SEQ ID NO: 23 - DNA, 53 bp, Artificial Sequence
  Synthetic
taatacgactcactatagggtggaggcccggaggccgttttagagctagaaat

SEQ ID NO: 24 - DNA, 54 bp, Artificial Sequence
  Synthetic
taatacgactcactatagggtggaggcccggaggccggttttagagctagaaat

SEQ ID NO: 25 - DNA, 54 bp, Artificial Sequence
  Synthetic
taatacgactcactatagtctgaagatccccggcctcgttttagagctagaaat

SEQ ID NO: 26 - DNA, 54 bp, Artificial Sequence
  Synthetic
taatacgactcactatagtgggtggaggcccggaggcgttttagagctagaaat

SEQ ID NO: 27 - DNA, 54 bp, Artificial Sequence
  Synthetic
taatacgactcactatagctgaagatccccggcctccgttttagagctagaaat

SEQ ID NO: 28 - DNA, 54 bp, Artificial Sequence
  Synthetic
taatacgactcactatagacaatgccacgcttctgccgttttagagctagaaat

SEQ ID NO: 29 - DNA, 54 bp, Artificial Sequence
  Synthetic
taatacgactcactatagcttcagaccttggcactgggttttagagctagaaat

SEQ ID NO: 30 - DNA, 54 bp, Artificial Sequence
  Synthetic
taatacgactcactatagggcacgggcagcttgccgggttttagagctagaaat

SEQ ID NO: 31 - DNA, 54 bp, Artificial Sequence
  Synthetic
taatacgactcactatagggcacgggcagcttgccgcgttttagagctagaaat

SEQ ID NO: 32 - DNA, 54 bp, Artificial Sequence
  Synthetic
taatacgactcactatagggcacgggcagcttgcccggttttagagctagaaat

SEQ ID NO: 33 - DNA, 54 bp, Artificial Sequence
  Synthetic
taatacgactcactatagggcacgggcagcttcccgggttttagagctagaaat

SEQ ID NO: 34 - DNA, 200 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtgaagc

SEQ ID NO: 35 - DNA, 183 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgc

SEQ ID NO: 36 - DNA, 198 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtgaa

SEQ ID NO: 37 - DNA, 196 bp, Artificial Sequence
  Synthetic
tcttctaggtatgacaacgaatttggctacagcaacagggtggtggacctcatggcccacatggcctccaaggaggtgagcggctggcggctgttcaagaagattagctaagacccctggaccaccagccccagcaagagcacaagaggaagagagagaccctcactgctggggagtccctgcgacgatgagagtg

SEQ ID NO: 38 - DNA, 196 bp, Artificial Sequence
  Synthetic
cagctcagagccctgtggcggactacagggcctgcacagacggtcactcaaagaaagatgtccctgtgccctagctaatcttcttgaacagccgccagccgctcacctccttggcgatggcaaagggcttctccacctcgatcttgccgcagtctgcgatgatcacatccttcaggggtgacgatgagagtgaagc

SEQ ID NO: 39 - DNA, 179 bp, Artificial Sequence
  Synthetic
cagctcagagccctgtggcggactacagggcctgcacagacggtcactcaaagaaagatgtccctgtgccctagctaatcttcttgaacagccgccagccgctcacctccttggcgatggcaaagggcttctccacctcgatcttgccgcagtctgcgatgatcacatccttcaggggt

SEQ ID NO: 40 - DNA, 200 bp, Artificial Sequence
  Synthetic
gaggtcaaggaccgctgcaccctggcagagaagctggggggcagtgccgtcatctccctggagggcaagcctttggtgagcggctggcggctgttcaagaagattagctgagccccttctggccccctgcctggagcatctggcagccccacacctgcccttgggggttgcaggctgccccctgacgatgagagtgaagc

SEQ ID NO: 41 - DNA, 183 bp, Artificial Sequence
  Synthetic
gaggtcaaggaccgctgcaccctggcagagaagctggggggcagtgccgtcatctccctggagggcaagcctttggtgagcggctggcggctgttcaagaagattagctgagccccttctggccccctgcctggagcatctggcagccccacacctgcccttgggggttgcaggctgccccct

SEQ ID NO: 42 - DNA, 185 bp, Artificial Sequence
  Synthetic
ggaggctctgtacctggtgtgtggggaacgtggtttcttctacacacccaagtcccgtcgtgaagtggagaagcgtgtgagcggctggcggctgttcaagaagattagcaagcgtgacccgcaagtgccacaactggagctgggtggaggcccggaggccggggatcttcagaccttggcactgg

SEQ ID NO: 43 - DNA, 183 bp, Artificial Sequence
  Synthetic
acacccaagtcccgtcgtgaagtggaggacccgcaagtgccacaactggagctgggtggaggcccggagaagcgtgtgagcggctggcggctgttcaagaagattagcaagcgtgccggggatcttcagaccttggcactggaggttgcccggcagaagcgtggcattgtggatcagtgctgc

SEQ ID NO: 44 - DNA, 200 bp, Artificial Sequence
  Synthetic
acacccaagtcccgtcgtgaagtggaggacccgcaagtgccacaactggagctgggtggaggcccggagaagcgtgtgagcggctggcggctgttcaagaagattagcaagcgtgccggggatcttcagaccttggcactggaggttgcccggcagaagcgtggcattgtggatcagtgctgcgacgatgagagtgaagc

SEQ ID NO: 45 - DNA, 185 bp, Artificial Sequence
  Synthetic
gcaagtgccacaactggagctgggtggaggcccggaggccggggatcttcagaccttggcactggaggttaagcgtgtgagcggctggcggctgttcaagaagattagcaagcgtgcccggcagaagcgtggcattgtggatcagtgctgcaccagcatctgctccctctaccaactggagaact

SEQ ID NO: 46 - DNA, 200 bp, Artificial Sequence
  Synthetic
gacaacgaatttggctacagcaacagggtggtggacctcatggcccacatggcctccaaggagggtggcggccgtgaccacatggtccttcatgagtatgtaaatgctgctgggattacataagacccctggaccaccagccccagcaagagcacaagaggaagagagagaccctcactgctggacgatgagagtgaagc

SEQ ID NO: 47 - DNA, 183 bp, Artificial Sequence
  Synthetic
gacaacgaatttggctacagcaacagggtggtggacctcatggcccacatggcctccaaggagggtggcggccgtgaccacatggtccttcatgagtatgtaaatgctgctgggattacataagacccctggaccaccagccccagcaagagcacaagaggaagagagagaccctcactgctg

SEQ ID NO: 48 - DNA, 137 bp, Artificial Sequence
  Synthetic
gtgggaagagctgcaggaggtgaagctgggagtgtgggacctcggtgagagtgaccggctcaccggactactagaccgcggcctttctgggccatatctgtgagggagccaaggagcagggacgatgagagtgaagc

SEQ ID NO: 49 - DNA, 93 bp, Artificial Sequence
  Synthetic
cagatatggcccagaaaggccgcggtctagtagtccggtgagccggtcactgtccccgaggtcccacacacccagcgacgatgagagtgaagc

SEQ ID NO: 50 - DNA, 831 bp, Artificial Sequence
  Synthetic
taatacgactcactatagcttcactctcatcgtcggctttattcattgcagaggggtgggcggggagtggtggactcagttgcagtagttctccagttggtagagggagcagatgctggtgcagcactgatccacaatgccacgcttctgccgggcaacctccagtgccaaggtctgaagatccccggcacgcttatttttcattttgagtgtcacgtaggcttctatgcagttgatgaagatgtcaaactcattcatggccttgtagacacctttgtcttggagcttattaaaatcattcttcacctgctccactgccttgcttttattctcacaggggagaaatcgatgacagcgtcgcagctgtatccagagggtcttcagcttctctcccagggaattcaaatgctccttgatttctgggccatggttctctgcctggggcatcacttctaccaggtaaaacttgatcatttctgacaaggcttggcaacccaagtaacccttaaagtcctgcagtaaggaatctgtcagcagtatgttgtccagctggtccttcttttgaaagaaagtcttcacttgactgaaggcagccctcagctctcggagcatgtgggtctggctgactgggaagtgggtgcagttattgtcaccccggatggaatggcctttgctacgcttctccgggcctccacccagctccagttgtggcacttgcgggtcctccacttcacgacgggacttgggtgtgtagaagaaaccacgttccccacacaccaggtacagagcctccaccaggtgaggaccacaaaggtgctgtttgacaaaagc

SEQ ID NO: 51 - DNA, 814 bp, Artificial Sequence
  Synthetic
taatacgactcactataggctttattcattgcagaggggtgggcggggagtggtggactcagttgcagtagttctccagttggtagagggagcagatgctggtgcagcactgatccacaatgccacgcttctgccgggcaacctccagtgccaaggtctgaagatccccggcacgcttatttttcattttgagtgtcacgtaggcttctatgcagttgatgaagatgtcaaactcattcatggccttgtagacacctttgtcttggagcttattaaaatcattcttcacctgctccactgccttgcttttattctcacaggggagaaatcgatgacagcgtcgcagctgtatccagagggtcttcagcttctctcccagggaattcaaatgctccttgatttctgggccatggttctctgcctggggcatcacttctaccaggtaaaacttgatcatttctgacaaggcttggcaacccaagtaacccttaaagtcctgcagtaaggaatctgtcagcagtatgttgtccagctggtccttcttttgaaagaaagtcttcacttgactgaaggcagccctcagctctcggagcatgtgggtctggctgactgggaagtgggtgcagttattgtcaccccggatggaatggcctttgctacgcttctccgggcctccacccagctccagttgtggcacttgcgggtcctccacttcacgacgggacttgggtgtgtagaagaaaccacgttccccacacaccaggtacagagcctccaccaggtgaggaccacaaaggtgctgtttgacaaaagc

SEQ ID NO: 52 - DNA, 32 bp, Artificial Sequence
  Synthetic
taatacgactcactatagcttcactctcatcg

SEQ ID NO: 53 - DNA, 42 bp, Artificial Sequence
  Synthetic
taatacgactcactataggctttattcattgcagaggggtgg

SEQ ID NO: 54 - DNA, 21 bp, Artificial Sequence
  Synthetic
gcttttgtcaaacagcacctt

SEQ ID NO: 55 - DNA, 814 bp, Artificial Sequence
  Synthetic
gcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggagaagcgtagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaataagcgtgccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagccgacgatgagagtgaagc

SEQ ID NO: 56 - DNA, 797 bp, Artificial Sequence
  Synthetic
gcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggagaagcgtagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaataagcgtgccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagcc